SEQUENCE LISTING (75 sequences)

SEQ ID NO: 1 - protein, 1159 aa, Homo sapiens
  MISC_FEATURE (1)..(1159): SorCS2 with intracellular tail A
MAHRGPSRASKGPGPTARAPSPGAPPPPRSPRSRPLLLLLLLLGACGAAGRSPEPGRLGPHAQLTRVPRSPPAGRAEPGGGEDRQARGTEPGAPGPSPGPAPGPGEDGAPAAGYRRWERAAPLAGVASRAQVSLISTSFVLKGDATHNQAMVHWTGENSSVILILTKYYHADMGKVLESSLWRSSDFGTSYTKLTLQPGVTTVIDNFYICPTNKRKVILVSSSLSDRDQSLFLSADEGATFQKQPIPFFVETLIFHPKEEDKVLAYTKESKLYVSSDLGKKWTLLQERVTKDHVFWSVSGVDADPDLVHVEAQDLGGDFRYVTCAIHNCSEKMLTAPFAGPIDHGSLTVQDDYIFFKATSANQTKYYVSYRRNEFVLMKLPKYALPKDLQIISTDESQVFVAVQEWYQMDTYNLYQSDPRGVRYALVLQDVRSSRQAEESVLIDILEVRGVKGVFLANQKIDGKVMTLITYNKGRDWDYLRPPSMDMNGKPTNCKPPDCHLHLHLRWADNPYVSGTVHTKDTAPGLIMGAGNLGSQLVEYKEEMYITSDCGHTWRQVFEEEHHILYLDHGGVIVAIKDTSIPLKILKFSVDEGLTWSTHNFTSTSVFVDGLLSEPGDETLVMTVFGHISFRSDWELVKVDFRPSFSRQCGEEDYSSWELSNLQGDRCIMGQQRSFRKRKSTSWCIKGRSFTSALTSRVCECRDSDFLCDYGFERSSSSESSTNKCSANFWFNPLSPPDDCALGQTYTSSLGYRKVVSNVCEGGVDMQQSQVQLQCPLTPPRGLQVSIQGEAVAVRPGEDVLFVVRQEQGDVLTTKYQVDLGDGFKAMYVNLTLTGEPIRHRYESPGIYRVSVRAENTAGHDEAVLFVQVNSPLQALYLEVVPVIGLNQEVNLTAVLLPLNPNLTVFYWWIGHSLQPLLSLDNSVTTRFSDTGDVRVTVQAACGNSVLQDSRVLRVLDQFQVMPLQFSKELDAYNPNTPEWREDVGLVVTRLLSKETSVPQELLVTVVKPGLPTLADLYVLLPPPRPTRKRSLSSDKRLAAIQQVLNAQKISFLLRGGVRVLVALRDTGTGAEQLGGGGGYWAVVVLFVIGLFAAGAFILYKFKRKRPGRTVYAQMHNEKEQEMTSPVSHSEDVQGAVQGNHSGVVLSINSREMHSYLVS

SEQ ID NO: 2 - protein, 1159 aa, Artificial sequence
  peptide
  MISC_FEATURE (1100)..(1159): SorCS2 intracellular tail A X in positions 1125, 1128 and 1130 independently chosen from S, E, D or J with the provisio that not all are S.
MAHRGPSRASKGPGPTARAPSPGAPPPPRSPRSRPLLLLLLLLGACGAAGRSPEPGRLGPHAQLTRVPRSPPAGRAEPGGGEDRQARGTEPGAPGPSPGPAPGPGEDGAPAAGYRRWERAAPLAGVASRAQVSLISTSFVLKGDATHNQAMVHWTGENSSVILILTKYYHADMGKVLESSLWRSSDFGTSYTKLTLQPGVTTVIDNFYICPTNKRKVILVSSSLSDRDQSLFLSADEGATFQKQPIPFFVETLIFHPKEEDKVLAYTKESKLYVSSDLGKKWTLLQERVTKDHVFWSVSGVDADPDLVHVEAQDLGGDFRYVTCAIHNCSEKMLTAPFAGPIDHGSLTVQDDYIFFKATSANQTKYYVSYRRNEFVLMKLPKYALPKDLQIISTDESQVFVAVQEWYQMDTYNLYQSDPRGVRYALVLQDVRSSRQAEESVLIDILEVRGVKGVFLANQKIDGKVMTLITYNKGRDWDYLRPPSMDMNGKPTNCKPPDCHLHLHLRWADNPYVSGTVHTKDTAPGLIMGAGNLGSQLVEYKEEMYITSDCGHTWRQVFEEEHHILYLDHGGVIVAIKDTSIPLKILKFSVDEGLTWSTHNFTSTSVFVDGLLSEPGDETLVMTVFGHISFRSDWELVKVDFRPSFSRQCGEEDYSSWELSNLQGDRCIMGQQRSFRKRKSTSWCIKGRSFTSALTSRVCECRDSDFLCDYGFERSSSSESSTNKCSANFWFNPLSPPDDCALGQTYTSSLGYRKVVSNVCEGGVDMQQSQVQLQCPLTPPRGLQVSIQGEAVAVRPGEDVLFVVRQEQGDVLTTKYQVDLGDGFKAMYVNLTLTGEPIRHRYESPGIYRVSVRAENTAGHDEAVLFVQVNSPLQALYLEVVPVIGLNQEVNLTAVLLPLNPNLTVFYWWIGHSLQPLLSLDNSVTTRFSDTGDVRVTVQAACGNSVLQDSRVLRVLDQFQVMPLQFSKELDAYNPNTPEWREDVGLVVTRLLSKETSVPQELLVTVVKPGLPTLADLYVLLPPPRPTRKRSLSSDKRLAAIQQVLNAQKISFLLRGGVRVLVALRDTGTGAEQLGGGGGYWAVVVLFVIGLFAAGAFILYKFKRKRPGRTVYAQMHNEKEQEMTXPVXHXEDVQGAVQGNHSGVVLSINSREMHSYLVS

SEQ ID NO: 3 - protein, 1173 aa, Homo sapiens
  MISC_FEATURE (1)..(1173): SorCS2 with intracellular tail B
MAHRGPSRASKGPGPTARAPSPGAPPPPRSPRSRPLLLLLLLLGACGAAGRSPEPGRLGPHAQLTRVPRSPPAGRAEPGGGEDRQARGTEPGAPGPSPGPAPGPGEDGAPAAGYRRWERAAPLAGVASRAQVSLISTSFVLKGDATHNQAMVHWTGENSSVILILTKYYHADMGKVLESSLWRSSDFGTSYTKLTLQPGVTTVIDNFYICPTNKRKVILVSSSLSDRDQSLFLSADEGATFQKQPIPFFVETLIFHPKEEDKVLAYTKESKLYVSSDLGKKWTLLQERVTKDHVFWSVSGVDADPDLVHVEAQDLGGDFRYVTCAIHNCSEKMLTAPFAGPIDHGSLTVQDDYIFFKATSANQTKYYVSYRRNEFVLMKLPKYALPKDLQIISTDESQVFVAVQEWYQMDTYNLYQSDPRGVRYALVLQDVRSSRQAEESVLIDILEVRGVKGVFLANQKIDGKVMTLITYNKGRDWDYLRPPSMDMNGKPTNCKPPDCHLHLHLRWADNPYVSGTVHTKDTAPGLIMGAGNLGSQLVEYKEEMYITSDCGHTWRQVFEEEHHILYLDHGGVIVAIKDTSIPLKILKFSVDEGLTWSTHNFTSTSVFVDGLLSEPGDETLVMTVFGHISFRSDWELVKVDFRPSFSRQCGEEDYSSWELSNLQGDRCIMGQQRSFRKRKSTSWCIKGRSFTSALTSRVCECRDSDFLCDYGFERSSSSESSTNKCSANFWFNPLSPPDDCALGQTYTSSLGYRKVVSNVCEGGVDMQQSQVQLQCPLTPPRGLQVSIQGEAVAVRPGEDVLFVVRQEQGDVLTTKYQVDLGDGFKAMYVNLTLTGEPIRHRYESPGIYRVSVRAENTAGHDEAVLFVQVNSPLQALYLEVVPVIGLNQEVNLTAVLLPLNPNLTVFYWWIGHSLQPLLSLDNSVTTRFSDTGDVRVTVQAACGNSVLQDSRVLRVLDQFQVMPLQFSKELDAYNPNTPEWREDVGLVVTRLLSKETSVPQELLVTVVKPGLPTLADLYVLLPPPRPTRKRSLSSDKRLAAIQQVLNAQKISFLLRGGVRVLVALRDTGTGAEQLGGGGGYWAVVVLFVIGLFAAGAFILYKFKRKRPGRTVYAQMHNEKEQEMTSPVSHSEDVQGAVQEEFIDDDLDSQTLGGNHSGVVLSINSREMHSYLVS

SEQ ID NO: 4 - protein, 1172 aa, Artificial sequence
  peptide
  MISC_FEATURE (1100)..(1172): SorCS2 intracellular tail B X in positions 1125, 1128 and 1130 independently chosen from S, E, D or J with the provisio that not all are S.
MAHRGPSRASKGPGPTARAPSPGAPPPPRSPRSRPLLLLLLLLGACGAAGRSPEPGRLGPHAQLTRVPRSPPAGRAEPGGGEDRQARGTEPGAPGPSPGPAPGPGEDGAPAAGYRRWERAAPLAGVASRAQVSLISTSFVLKGDATHNQAMVHWTGENSSVILILTKYYHADMGKVLESSLWRSSDFGTSYTKLTLQPGVTTVIDNFYICPTNKRKVILVSSSLSDRDQSLFLSADEGATFQKQPIPFFVETLIFHPKEEDKVLAYTKESKLYVSSDLGKKWTLLQERVTKDHVFWSVSGVDADPDLVHVEAQDLGGDFRYVTCAIHNCSEKMLTAPFAGPIDHGSLTVQDDYIFFKATSANQTKYYVSYRRNEFVLMKLPKYALPKDLQIISTDESQVFVAVQEWYQMDTYNLYQSDPRGVRYALVLQDVRSSRQAEESVLIDILEVRGVKGVFLANQKIDGKVMTLITYNKGRDWDYLRPPSMDMNGKPTNCKPPDCHLHLHLRWADNPYVSGTVHTKDTAPGLIMGAGNLGSQLVEYKEEMYITSDCGHTWRQVFEEEHHILYLDHGGVIVAIKDTSIPLKILKFSVDEGLTWSTHNFTSTSVFVDGLLSEPGDETLVMTVFGHISFRSDWELVKVDFRPSFSRQCGEEDYSSWELSNLQGDRCIMGQQRSFRKRKSTSWCIKGRSFTSALTSRVCECRDSDFLCDYGFERSSSSESSTNKCSANFWFNPLSPPDDCALGQTYTSSLGYRKVVSNVCEGGVDMQQSQVQLQCPLTPPRGLQVSIQGEAVAVRPGEDVLFVVRQEQGDVLTTKYQVDLGDGFKAMYVNLTLTGEPIRHRYESPGIYRVSVRAENTAGHDEAVLFVQVNSPLQALYLEVVPVIGLNQEVNLTAVLLPLNPNLTVFYWWIGHSLQPLLSLDNSVTTRFSDTGDVRVTVQAACGNSVLQDSRVLRVLDQFQVMPLQFSKELDAYNPNTPEWREDVGLVVTRLLSKETSVPQELLVTVVKPGLPTLADLYVLLPPPRPTRKRSLSSDKRLAAIQQVLNAQKISFLLRGGVRVLVALRDTGTGAEQLGGGGGYWAVVVLFVIGLFAAGAFILYKFKRKRPGRTVYAQMHNEKEQEMTXPVXHXEDQGAVQEEFIDDDLDSQTLGGNHSGVVLSINSREMHSYLVS

SEQ ID NO: 5 - protein, 1174 aa, Homo sapiens
  MISC_FEATURE (1)..(1174): SorCS2 with intracellular tail C
MAHRGPSRASKGPGPTARAPSPGAPPPPRSPRSRPLLLLLLLLGACGAAGRSPEPGRLGPHAQLTRVPRSPPAGRAEPGGGEDRQARGTEPGAPGPSPGPAPGPGEDGAPAAGYRRWERAAPLAGVASRAQVSLISTSFVLKGDATHNQAMVHWTGENSSVILILTKYYHADMGKVLESSLWRSSDFGTSYTKLTLQPGVTTVIDNFYICPTNKRKVILVSSSLSDRDQSLFLSADEGATFQKQPIPFFVETLIFHPKEEDKVLAYTKESKLYVSSDLGKKWTLLQERVTKDHVFWSVSGVDADPDLVHVEAQDLGGDFRYVTCAIHNCSEKMLTAPFAGPIDHGSLTVQDDYIFFKATSANQTKYYVSYRRNEFVLMKLPKYALPKDLQIISTDESQVFVAVQEWYQMDTYNLYQSDPRGVRYALVLQDVRSSRQAEESVLIDILEVRGVKGVFLANQKIDGKVMTLITYNKGRDWDYLRPPSMDMNGKPTNCKPPDCHLHLHLRWADNPYVSGTVHTKDTAPGLIMGAGNLGSQLVEYKEEMYITSDCGHTWRQVFEEEHHILYLDHGGVIVAIKDTSIPLKILKFSVDEGLTWSTHNFTSTSVFVDGLLSEPGDETLVMTVFGHISFRSDWELVKVDFRPSFSRQCGEEDYSSWELSNLQGDRCIMGQQRSFRKRKSTSWCIKGRSFTSALTSRVCECRDSDFLCDYGFERSSSSESSTNKCSANFWFNPLSPPDDCALGQTYTSSLGYRKVVSNVCEGGVDMQQSQVQLQCPLTPPRGLQVSIQGEAVAVRPGEDVLFVVRQEQGDVLTTKYQVDLGDGFKAMYVNLTLTGEPIRHRYESPGIYRVSVRAENTAGHDEAVLFVQVNSPLQALYLEVVPVIGLNQEVNLTAVLLPLNPNLTVFYWWIGHSLQPLLSLDNSVTTRFSDTGDVRVTVQAACGNSVLQDSRVLRVLDQFQVMPLQFSKELDAYNPNTPEWREDVGLVVTRLLSKETSVPQELLVTVVKPGLPTLADLYVLLPPPRPTRKRSLSSDKRLAAIQQVLNAQKISFLLRGGVRVLVALRDTGTGAEQLGGGGGYWAVVVLFVIGLFAAGAFILYKFKSRKRPGRTVYAQMHNEKEQEMTSPVSHSEDVQGAVQEEFIDDDLDSQTLGGNHSGVVLSINSREMHSYLVS

SEQ ID NO: 6 - protein, 1173 aa, Artificial sequence
  peptide
  MISC_FEATURE (1100)..(1173): SorCS2 intracellular tail C X in positions 1125, 1128 and 1130 independently chosen from S, E, D or J with the provisio that not all are S.
MAHRGPSRASKGPGPTARAPSPGAPPPPRSPRSRPLLLLLLLLGACGAAGRSPEPGRLGPHAQLTRVPRSPPAGRAEPGGGEDRQARGTEPGAPGPSPGPAPGPGEDGAPAAGYRRWERAAPLAGVASRAQVSLISTSFVLKGDATHNQAMVHWTGENSSVILILTKYYHADMGKVLESSLWRSSDFGTSYTKLTLQPGVTTVIDNFYICPTNKRKVILVSSSLSDRDQSLFLSADEGATFQKQPIPFFVETLIFHPKEEDKVLAYTKESKLYVSSDLGKKWTLLQERVTKDHVFWSVSGVDADPDLVHVEAQDLGGDFRYVTCAIHNCSEKMLTAPFAGPIDHGSLTVQDDYIFFKATSANQTKYYVSYRRNEFVLMKLPKYALPKDLQIISTDESQVFVAVQEWYQMDTYNLYQSDPRGVRYALVLQDVRSSRQAEESVLIDILEVRGVKGVFLANQKIDGKVMTLITYNKGRDWDYLRPPSMDMNGKPTNCKPPDCHLHLHLRWADNPYVSGTVHTKDTAPGLIMGAGNLGSQLVEYKEEMYITSDCGHTWRQVFEEEHHILYLDHGGVIVAIKDTSIPLKILKFSVDEGLTWSTHNFTSTSVFVDGLLSEPGDETLVMTVFGHISFRSDWELVKVDFRPSFSRQCGEEDYSSWELSNLQGDRCIMGQQRSFRKRKSTSWCIKGRSFTSALTSRVCECRDSDFLCDYGFERSSSSESSTNKCSANFWFNPLSPPDDCALGQTYTSSLGYRKVVSNVCEGGVDMQQSQVQLQCPLTPPRGLQVSIQGEAVAVRPGEDVLFVVRQEQGDVLTTKYQVDLGDGFKAMYVNLTLTGEPIRHRYESPGIYRVSVRAENTAGHDEAVLFVQVNSPLQALYLEVVPVIGLNQEVNLTAVLLPLNPNLTVFYWWIGHSLQPLLSLDNSVTTRFSDTGDVRVTVQAACGNSVLQDSRVLRVLDQFQVMPLQFSKELDAYNPNTPEWREDVGLVVTRLLSKETSVPQELLVTVVKPGLPTLADLYVLLPPPRPTRKRSLSSDKRLAAIQQVLNAQKISFLLRGGVRVLVALRDTGTGAEQLGGGGGYWAVVVLFVIGLFAAGAFILYKFKSRKRPGRTVYAQMHNEKEQEMTXPVXHXEDQGAVQEEFIDDDLDSQTLGGNHSGVVLSINSREMHSYLVS

SEQ ID NO: 7 - protein, 1160 aa, Homo sapiens
  MISC_FEATURE (1)..(1160): SorCS2 with intracellular tail D
MAHRGPSRASKGPGPTARAPSPGAPPPPRSPRSRPLLLLLLLLGACGAAGRSPEPGRLGPHAQLTRVPRSPPAGRAEPGGGEDRQARGTEPGAPGPSPGPAPGPGEDGAPAAGYRRWERAAPLAGVASRAQVSLISTSFVLKGDATHNQAMVHWTGENSSVILILTKYYHADMGKVLESSLWRSSDFGTSYTKLTLQPGVTTVIDNFYICPTNKRKVILVSSSLSDRDQSLFLSADEGATFQKQPIPFFVETLIFHPKEEDKVLAYTKESKLYVSSDLGKKWTLLQERVTKDHVFWSVSGVDADPDLVHVEAQDLGGDFRYVTCAIHNCSEKMLTAPFAGPIDHGSLTVQDDYIFFKATSANQTKYYVSYRRNEFVLMKLPKYALPKDLQIISTDESQVFVAVQEWYQMDTYNLYQSDPRGVRYALVLQDVRSSRQAEESVLIDILEVRGVKGVFLANQKIDGKVMTLITYNKGRDWDYLRPPSMDMNGKPTNCKPPDCHLHLHLRWADNPYVSGTVHTKDTAPGLIMGAGNLGSQLVEYKEEMYITSDCGHTWRQVFEEEHHILYLDHGGVIVAIKDTSIPLKILKFSVDEGLTWSTHNFTSTSVFVDGLLSEPGDETLVMTVFGHISFRSDWELVKVDFRPSFSRQCGEEDYSSWELSNLQGDRCIMGQQRSFRKRKSTSWCIKGRSFTSALTSRVCECRDSDFLCDYGFERSSSSESSTNKCSANFWFNPLSPPDDCALGQTYTSSLGYRKVVSNVCEGGVDMQQSQVQLQCPLTPPRGLQVSIQGEAVAVRPGEDVLFVVRQEQGDVLTTKYQVDLGDGFKAMYVNLTLTGEPIRHRYESPGIYRVSVRAENTAGHDEAVLFVQVNSPLQALYLEVVPVIGLNQEVNLTAVLLPLNPNLTVFYWWIGHSLQPLLSLDNSVTTRFSDTGDVRVTVQAACGNSVLQDSRVLRVLDQFQVMPLQFSKELDAYNPNTPEWREDVGLVVTRLLSKETSVPQELLVTVVKPGLPTLADLYVLLPPPRPTRKRSLSSDKRLAAIQQVLNAQKISFLLRGGVRVLVALRDTGTGAEQLGGGGGYWAVVVLFVIGLFAAGAFILYKFKSRKRPGRTVYAQMHNEKEQEMTSPVSHSEDVQGAVQGNHSGVVLSINSREMHSYLVS

SEQ ID NO: 8 - protein, 1159 aa, Artificial sequence
  peptide
  MISC_FEATURE (1100)..(1159): SorCS2 intracellular tail D X in positions 1125, 1128 and 1130 independently chosen from S, E, D or J with the provisio that not all are S.
MAHRGPSRASKGPGPTARAPSPGAPPPPRSPRSRPLLLLLLLLGACGAAGRSPEPGRLGPHAQLTRVPRSPPAGRAEPGGGEDRQARGTEPGAPGPSPGPAPGPGEDGAPAAGYRRWERAAPLAGVASRAQVSLISTSFVLKGDATHNQAMVHWTGENSSVILILTKYYHADMGKVLESSLWRSSDFGTSYTKLTLQPGVTTVIDNFYICPTNKRKVILVSSSLSDRDQSLFLSADEGATFQKQPIPFFVETLIFHPKEEDKVLAYTKESKLYVSSDLGKKWTLLQERVTKDHVFWSVSGVDADPDLVHVEAQDLGGDFRYVTCAIHNCSEKMLTAPFAGPIDHGSLTVQDDYIFFKATSANQTKYYVSYRRNEFVLMKLPKYALPKDLQIISTDESQVFVAVQEWYQMDTYNLYQSDPRGVRYALVLQDVRSSRQAEESVLIDILEVRGVKGVFLANQKIDGKVMTLITYNKGRDWDYLRPPSMDMNGKPTNCKPPDCHLHLHLRWADNPYVSGTVHTKDTAPGLIMGAGNLGSQLVEYKEEMYITSDCGHTWRQVFEEEHHILYLDHGGVIVAIKDTSIPLKILKFSVDEGLTWSTHNFTSTSVFVDGLLSEPGDETLVMTVFGHISFRSDWELVKVDFRPSFSRQCGEEDYSSWELSNLQGDRCIMGQQRSFRKRKSTSWCIKGRSFTSALTSRVCECRDSDFLCDYGFERSSSSESSTNKCSANFWFNPLSPPDDCALGQTYTSSLGYRKVVSNVCEGGVDMQQSQVQLQCPLTPPRGLQVSIQGEAVAVRPGEDVLFVVRQEQGDVLTTKYQVDLGDGFKAMYVNLTLTGEPIRHRYESPGIYRVSVRAENTAGHDEAVLFVQVNSPLQALYLEVVPVIGLNQEVNLTAVLLPLNPNLTVFYWWIGHSLQPLLSLDNSVTTRFSDTGDVRVTVQAACGNSVLQDSRVLRVLDQFQVMPLQFSKELDAYNPNTPEWREDVGLVVTRLLSKETSVPQELLVTVVKPGLPTLADLYVLLPPPRPTRKRSLSSDKRLAAIQQVLNAQKISFLLRGGVRVLVALRDTGTGAEQLGGGGGYWAVVVLFVIGLFAAGAFILYKFKSRKRPGRTVYAQMHNEKEQEMTXPVXHXEDQGAVQGNHSGVVLSINSREMHSYLVS

SEQ ID NO: 9 - protein, 8 aa, Artificial sequence
  peptide
  MISC_FEATURE (1)..(1): Xaa=X1 is Thr or Ile
  MISC_FEATURE (1)..(8): X1, X2, X3, X4, X5, X6 are independently chosen as defined, with the proviso that X2, X4 and X6 can be any amino acid with the further proviso that at least one of X2, X4 and X6 is not Ser
  MISC_FEATURE (2)..(2): Xaa=X2
  MISC_FEATURE (3)..(3): Xaa=X3 is Pro or Ser
  MISC_FEATURE (5)..(5): Xaa=X4
  MISC_FEATURE (6)..(6): Xaa=X5 is His or Gln
  MISC_FEATURE (7)..(7): Xaa=X6
XXXVXXXE

SEQ ID NO: 10 - protein, 13 aa, Artificial sequence
  peptide
  MISC_FEATURE (1)..(13): X1, X2, X3, X4, X5, X6 are independently chosen as defined, with the proviso that X2, X4 and X6 can be any amino acid with the further proviso that at least one of X2, X4 and X6 is not Ser
  MISC_FEATURE (6)..(6): Xaa=X1=Thr or Ile
  MISC_FEATURE (7)..(7): Xaa=X2
  MISC_FEATURE (8)..(8): Xaa=X3=Pro or Ser
  MISC_FEATURE (10)..(10): Xaa=X4
  MISC_FEATURE (11)..(11): Xaa=X5=His or Gln
  MISC_FEATURE (12)..(12): Xaa=X6
KEQEMXXXVXXXE

SEQ ID NO: 11 - protein, 16 aa, Artificial sequence
  peptide
  MISC_FEATURE (1)..(16): X1, X2, X3, X4, X5, X6, X7, X8, X9, X10, X11, X12, X13, X14 are independently chosen as defined with the proviso that X2, X4 and X6 can be any amino acid with the further proviso that at least one of X2, X4 and X6 is not Ser
  MISC_FEATURE (1)..(1): Xaa=X1=Thr or Ile
  MISC_FEATURE (2)..(2): Xaa=X2
  MISC_FEATURE (3)..(3): Xaa=X3=Pro or Ser
  MISC_FEATURE (5)..(5): Xaa=X4
  MISC_FEATURE (6)..(6): Xaa=X5=His or Gln
  MISC_FEATURE (7)..(7): Xaa=X6
  MISC_FEATURE (9)..(9): Xaa=X7=Asp, Asn, or Ser
  MISC_FEATURE (10)..(10): Xaa=X8=Val, Arg or Ala
  MISC_FEATURE (11)..(11): Xaa=X9=Pro or Gln
  MISC_FEATURE (12)..(12): Xaa=X10=Gly, Lys or Asn
  MISC_FEATURE (13)..(13): Xaa=X11=Ala, Ile or Val
  MISC_FEATURE (14)..(14): Xaa=X12=Val, Thr or Pro
  MISC_FEATURE (15)..(15): Xaa=X13=Gln or Leu
  MISC_FEATURE (16)..(16): Xaa=X14=Gly, Thr or Ser
XXXVXXXEXXXXXXXX

SEQ ID NO: 12 - protein, 21 aa, Artificial sequence
  peptide
  MISC_FEATURE (1)..(21): X1, X2, X3, X4, X5, X6, X7, X8, X9, X10, X11, X12, X13, X14 are independently chosen as defined with the proviso that X2, X4 and X6 can be any amino acid with the further proviso that at least one of X2, X4 and X6 is not Ser
  MISC_FEATURE (6)..(6): Xaa=X1=T or I
  MISC_FEATURE (7)..(7): Xaa=X2
  MISC_FEATURE (8)..(8): Xaa=X3=P or S
  MISC_FEATURE (10)..(10): Xaa=X4
  MISC_FEATURE (11)..(11): Xaa=X5=H or Q
  MISC_FEATURE (12)..(12): Xaa=X6
  MISC_FEATURE (14)..(14): Xaa=X7=D, N or S
  MISC_FEATURE (15)..(15): Xaa=X8=V, R or A
  MISC_FEATURE (16)..(16): Xaa=X9=P or Q
  MISC_FEATURE (17)..(17): Xaa=X10=G, K or N
  MISC_FEATURE (18)..(18): Xaa=X11=A, I or V
  MISC_FEATURE (19)..(19): Xaa=X12=V, T or P
  MISC_FEATURE (20)..(20): Xaa=X13=Q or L
  MISC_FEATURE (21)..(21): Xaa=X14=G, T or S
KEQEMXXXVXXXEXXXXXXXX

SEQ ID NO: 13 - protein, 8 aa, Artificial sequence
  peptide
  MISC_FEATURE (1)..(8): X2, X4, and X6 are independently chosen as any amino acid with the proviso that at least one of X2, X4 and X6 is not S
  MISC_FEATURE (2)..(2): Xaa=X2
  MISC_FEATURE (5)..(5): Xaa=X4
  MISC_FEATURE (7)..(7): Xaa=X6
TXPVXHXE

SEQ ID NO: 14 - protein, 8 aa, Artificial sequence
  peptide
  MISC_FEATURE (1)..(8): X2, X4, and X6 are independently chosen as any amino acid with the proviso that at least one of X2, X4 and X6 is not S
  MISC_FEATURE (2)..(2): Xaa=X2
  MISC_FEATURE (5)..(5): Xaa=X3
  MISC_FEATURE (7)..(7): Xaa=X5
IXPVXHXE

SEQ ID NO: 15 - protein, 8 aa, Artificial sequence
  peptide
  MISC_FEATURE (1)..(8): X2, X4, and X6 are independently chosen as any amino acid with the proviso that at least one of X2, X4 and X6 is not S
  MISC_FEATURE (2)..(2): Xaa=X2
  MISC_FEATURE (5)..(5): Xaa=X4
  MISC_FEATURE (7)..(7): Xaa=X6
IXSVXQXE

SEQ ID NO: 16 - protein, 13 aa, Artificial sequence
  peptide
  MISC_FEATURE (1)..(13): X2, X4, and X6 are independently chosen as any amino acid with the proviso that at least one of X2, X4 and X6 is not S
  MISC_FEATURE (7)..(7): Xaa=X2
  MISC_FEATURE (10)..(10): Xaa=X4
  MISC_FEATURE (12)..(12): Xaa=X6
KEQEMTXPVXHXE

SEQ ID NO: 17 - protein, 13 aa, Artificial sequence
  peptide
  MISC_FEATURE (1)..(13): X2, X4, and X6 are independently chosen as any amino acid with the proviso that at least one of X2, X4 and X6 is not S
  MISC_FEATURE (7)..(7): Xaa=X2
  MISC_FEATURE (10)..(10): Xaa=X4
  MISC_FEATURE (12)..(12): Xaa=X6
KEQEMIXPVXHXE

SEQ ID NO: 18 - protein, 13 aa, Artificial sequence
  peptide
  MISC_FEATURE (1)..(13): X2, X4, and X6 are independently chosen as any amino acid with the proviso that at least one of X2, X4 and X6 is not S
  MISC_FEATURE (7)..(7): Xaa=X2
  MISC_FEATURE (10)..(10): Xaa=X4
  MISC_FEATURE (12)..(12): Xaa=X6
KEQEMIXSVXQXE

SEQ ID NO: 19 - protein, 16 aa, Artificial sequence
  peptide
  MISC_FEATURE (1)..(16): X2, X4, and X6 are independently chosen as any amino acid with the proviso that at least one of X2, X4 and X6 is not S
  MISC_FEATURE (2)..(2): Xaa=X2
  MISC_FEATURE (5)..(5): Xaa=X4
  MISC_FEATURE (7)..(7): Xaa=X6
TXPVXHXEDVQGAVQG

SEQ ID NO: 20 - protein, 16 aa, Artificial sequence
  peptide
  MISC_FEATURE (1)..(16): X2, X4, and X6 are independently chosen as any amino acid with the proviso that at least one of X2, X4 and X6 is not S
  MISC_FEATURE (2)..(2): Xaa=X2
  MISC_FEATURE (5)..(5): Xaa=X4
  MISC_FEATURE (7)..(7): Xaa=X6
IXPVXHXESRPNVPQT

SEQ ID NO: 21 - protein, 16 aa, Artificial sequence
  peptide
  MISC_FEATURE (1)..(16): X2, X4, and X6 are independently chosen as any amino acid with the proviso that at least one of X2, X4 and X6 is not S
  MISC_FEATURE (2)..(2): Xaa=X2
  MISC_FEATURE (5)..(5): Xaa=X4
  MISC_FEATURE (7)..(7): Xaa=X6
IXSVXQXENAPKITLS

SEQ ID NO: 22 - protein, 21 aa, Artificial sequence
  peptide
  MISC_FEATURE (1)..(21): X2, X4, and X6 are independently chosen as any amino acid with the proviso that at least one of X2, X4 and X6 is not S
  MISC_FEATURE (7)..(7): Xaa=X2
  MISC_FEATURE (10)..(10): Xaa=X4
  MISC_FEATURE (12)..(12): Xaa=X6
KEQEMTXPVXHXEDVQGAVQG

SEQ ID NO: 23 - protein, 21 aa, Artificial sequence
  peptide
  MISC_FEATURE (1)..(21): X2, X4, and X6 are independently chosen as any amino acid with the proviso that at least one of X2, X4 and X6 is not S
  MISC_FEATURE (7)..(7): Xaa=X2
  MISC_FEATURE (10)..(10): Xaa=X4
  MISC_FEATURE (12)..(12): Xaa=X6
KEQEMIXPVXHXESRPNVPQT

SEQ ID NO: 24 - protein, 21 aa, Artificial sequence
  peptide
  MISC_FEATURE (1)..(21): X2, X4, and X6 are independently chosen as any amino acid with the proviso that at least one of X2, X4 and X6 is not S
  MISC_FEATURE (7)..(7): Xaa=X2
  MISC_FEATURE (10)..(10): Xaa=X4
  MISC_FEATURE (12)..(12): Xaa=X6
KEQEMIXSVXQXENAPKITLS

SEQ ID NO: 25 - protein, 8 aa, Artificial sequence
  peptide
  MISC_FEATURE (1)..(8): X2, X4, and X6 are independently chosen as any amino acid with the proviso that at least one of X2, X4 and X6 is not S and at least one of X2, X4 and X6 is selected from Asp, Glu and phosphoserine.
  MISC_FEATURE (2)..(2): Xaa=X2
  MISC_FEATURE (5)..(5): Xaa=X4
  MISC_FEATURE (7)..(7): Xaa=X6
TXPVXHXE

SEQ ID NO: 26 - protein, 8 aa, Artificial sequence
  peptide
  MISC_FEATURE (1)..(8): X2, X4, and X6 are independently chosen as any amino acid with the proviso that at least one of X2, X4 and X6 is not S and and at least one of X2, X4 and X6 is selected from Asp, Glu and phosphoserine.
  MISC_FEATURE (2)..(2): Xaa=X2
  MISC_FEATURE (5)..(5): Xaa=X4
  MISC_FEATURE (7)..(7): Xaa=X6
IXPVXHXE

SEQ ID NO: 27 - protein, 8 aa, Artificial sequence
  peptide
  MISC_FEATURE (1)..(8): X2, X4, and X6 are independently chosen as any amino acid with the proviso that at least one of X2, X4 and X6 is not S and and at least one of X2, X4 and X6 is selected from Asp, Glu and phosphoserine.
  MISC_FEATURE (2)..(2): Xaa=X2
  MISC_FEATURE (5)..(5): Xaa=X4
  MISC_FEATURE (7)..(7): Xaa=X6
IXSVXQXE

SEQ ID NO: 28 - protein, 13 aa, Artificial sequence
  peptide
  MISC_FEATURE (1)..(13): X2, X4, and X6 are independently chosen as any amino acid with the proviso that at least one of X2, X4 and X6 is not S and and at least one of X2, X4 and X6 is selected from Asp, Glu and phosphoserine.
  MISC_FEATURE (7)..(7): Xaa=X2
  MISC_FEATURE (10)..(10): Xaa=X4
  MISC_FEATURE (12)..(12): Xaa=X6
KEQEMTXPVXHXE

SEQ ID NO: 29 - protein, 13 aa, Artificial sequence
  peptide
  MISC_FEATURE (1)..(13): X2, X4, and X6 are independently chosen as any amino acid with the proviso that at least one of X2, X4 and X6 is not S and and at least one of X2, X4 and X6 is selected from Asp, Glu and phosphoserine.
  MISC_FEATURE (7)..(7): Xaa=X2
  MISC_FEATURE (10)..(10): Xaa=X4
  MISC_FEATURE (12)..(12): Xaa=X6
KEQEMIXPVXHXE

SEQ ID NO: 30 - protein, 13 aa, Artificial sequence
  peptide
  MISC_FEATURE (1)..(13): X2, X4, and X6 are independently chosen as any amino acid with the proviso that at least one of X2, X4 and X6 is not S and and at least one of X2, X4 and X6 is selected from Asp, Glu and phosphoserine.
  MISC_FEATURE (7)..(7): Xaa=X2
  MISC_FEATURE (10)..(10): Xaa=X4
  MISC_FEATURE (12)..(12): Xaa=X6
KEQEMIXSVXQXE

SEQ ID NO: 31 - protein, 16 aa, Artificial sequence
  peptide
  MISC_FEATURE (1)..(16): X2, X4, and X6 are independently chosen as any amino acid with the proviso that at least one of X2, X4 and X6 is not S and and at least one of X2, X4 and X6 is selected from Asp, Glu and phosphoserine.
  MISC_FEATURE (2)..(2): Xaa=X2
  MISC_FEATURE (5)..(5): Xaa=X4
  MISC_FEATURE (7)..(7): Xaa=X6
TXPVXHXEDVQGAVQG

SEQ ID NO: 32 - protein, 16 aa, Artificial sequence
  peptide
  MISC_FEATURE (1)..(16): X2, X4, and X6 are independently chosen as defined with the proviso that at least one of X2, X4 and X6 is not S and and at least one of X2, X4 and X6 is selected from Asp, Glu and phosphoserine.
  MISC_FEATURE (2)..(2): Xaa=X2
  MISC_FEATURE (5)..(5): Xaa=X4
  MISC_FEATURE (7)..(7): Xaa=X6
IXPVXHXESRPNVPQT

SEQ ID NO: 33 - protein, 16 aa, Artificial sequence
  peptide
  MISC_FEATURE (1)..(16): X2, X4, and X6 are independently chosen as any amino acid with the proviso that at least one of X2, X4 and X6 is not S and and at least one of X2, X4 and X6 is selected from Asp, Glu and phosphoserine.
  MISC_FEATURE (2)..(2): Xaa=X2
  MISC_FEATURE (5)..(5): Xaa=X4
  MISC_FEATURE (7)..(7): Xaa=X6
IXSVXQXENAPKITLS

SEQ ID NO: 34 - protein, 21 aa, Artificial sequence
  peptide
  MISC_FEATURE (1)..(21): X2, X4, and X6 are independently chosen as defined with the proviso that at least one of X2, X4 and X6 is not S and and at least one of X2, X4 and X6 is selected from Asp, Glu and phosphoserine.
  MISC_FEATURE (7)..(7): Xaa=X2
  MISC_FEATURE (10)..(10): Xaa=X4
  MISC_FEATURE (12)..(12): Xaa=X6
KEQEMTXPVXHXEDVQGAVQG

SEQ ID NO: 35 - protein, 21 aa, Artificial sequence
  peptide
  MISC_FEATURE (1)..(21): X2, X4, and X6 are independently chosen as any amino acid with the proviso that at least one of X2, X4 and X6 is not S and and at least one of X2, X4 and X6 is selected from Asp, Glu and phosphoserine.
  MISC_FEATURE (7)..(7): Xaa=X2
  MISC_FEATURE (10)..(10): Xaa=X4
  MISC_FEATURE (12)..(12): Xaa=X6
KEQEMIXPVXHXESRPNVPQT

SEQ ID NO: 36 - protein, 21 aa, Artificial sequence
  peptide
  MISC_FEATURE (1)..(21): X2, X4, and X6 are independently chosen as any amino acid with the proviso that at least one of X2, X4 and X6 is not S and iand at least one of X2, X4 and X6 is selected from Asp, Glu and phosphoserine.
  MISC_FEATURE (7)..(7): Xaa=X2
  MISC_FEATURE (10)..(10): Xaa=X4
  MISC_FEATURE (12)..(12): Xaa=X6
KEQEMIXSVXQXENAPKITLS

SEQ ID NO: 37 - protein, 8 aa, Artificial sequence
  peptide
TDPVDHDE

SEQ ID NO: 38 - protein, 13 aa, Artificial sequence
  peptide
KEQEMTDPVDHDE

SEQ ID NO: 39 - protein, 16 aa, Artificial sequence
  peptide
TDPVDHDEDVQGAVQG

SEQ ID NO: 40 - protein, 21 aa, Artificial sequence
  peptide
KEQEMTDPVDHDEDVQGAVQG

SEQ ID NO: 41 - protein, 32 aa, Artificial sequence
  peptide
YARAAARNARAEKEQEMTDPVDHDEDVQGAVQ

SEQ ID NO: 42 - protein, 8 aa, Artificial sequence
  peptide
IDPVDHDE

SEQ ID NO: 43 - protein, 13 aa, Artificial sequence
  peptide
KEQEMIDPVDHDE

SEQ ID NO: 44 - protein, 16 aa, Artificial sequence
  peptide
IDPVDHDESRPNVPQT

SEQ ID NO: 45 - protein, 21 aa, Artificial sequence
  peptide
KEQEMIDPVDHDESRPNVPQT

SEQ ID NO: 46 - protein, 8 aa, Artificial sequence
  peptide
IDSVDQDE

SEQ ID NO: 47 - protein, 13 aa, Artificial sequence
  peptide
KEQEMIDSVDQDE

SEQ ID NO: 48 - protein, 16 aa, Artificial sequence
  peptide
IDSVDQDENAPKITLS

SEQ ID NO: 49 - protein, 21 aa, Artificial sequence
  peptide
KEQEMIDSVDQDENAPKITLS

SEQ ID NO: 50 - protein, 8 aa, Artificial sequence
  peptide
  MISC_FEATURE (1)..(8): X2, X4, and X6 are independently chosen as any amino acid with the proviso that at least one of X2, X4 and X6 is not S and at least one of X2, X4 and X6 is selected from Ala, Gly, Leu, Ile, Val, Pro or Met.
  MISC_FEATURE (2)..(2): Xaa=X2
  MISC_FEATURE (5)..(5): Xaa=X4
  MISC_FEATURE (7)..(7): Xaa=X6
TXPVXHXE

SEQ ID NO: 51 - protein, 8 aa, Artificial sequence
  peptide
  MISC_FEATURE (1)..(8): X2, X4, and X6 are independently chosen as any amino acid with the proviso that at least one of X2, X4 and X6 is not S and at least one of X2, X4 and X6 is selected from Ala, Gly, Leu, Ile, Val, Pro or Met.
  MISC_FEATURE (2)..(2): Xaa=X2
  MISC_FEATURE (5)..(5): Xaa=X4
  MISC_FEATURE (7)..(7): Xaa=X6
IXPVXHXE

SEQ ID NO: 52 - protein, 8 aa, Artificial sequence
  peptide
  MISC_FEATURE (1)..(8): X2, X4, and X6 are independently chosen as any amino acid with the proviso that at least one of X2, X4 and X6 is not S and at least one of X2, X4 and X6 is selected from Ala, Gly, Leu, Ile, Val, Pro or Met.
  MISC_FEATURE (2)..(2): Xaa=X2
  MISC_FEATURE (5)..(5): Xaa=X4
  MISC_FEATURE (7)..(7): Xaa=X6
IXSVXQXE

SEQ ID NO: 53 - protein, 13 aa, Artificial sequence
  peptide
  MISC_FEATURE (1)..(13): X2, X4, and X6 are independently chosen as any amino acid with the proviso that at least one of X2, X4 and X6 is not S and at least one of X2, X4 and X6 is selected from Ala, Gly, Leu, Ile, Val, Pro or Met.
  MISC_FEATURE (7)..(7): Xaa=X2
  MISC_FEATURE (10)..(10): Xaa=X4
  MISC_FEATURE (12)..(12): Xaa=X6
KEQEMTXPVXHXE

SEQ ID NO: 54 - protein, 13 aa, Artificial sequence
  peptide
  MISC_FEATURE (1)..(13): X2, X4, and X6 are independently chosen as any amino acid with the proviso that at least one of X2, X4 and X6 is not S and at least one of X2, X4 and X6 is selected from Ala, Gly, Leu, Ile, Val, Pro or Met.
  MISC_FEATURE (7)..(7): Xaa=X2
  MISC_FEATURE (10)..(10): Xaa=X4
  MISC_FEATURE (12)..(12): Xaa=X6
KEQEMIXPVXHXE

SEQ ID NO: 55 - protein, 13 aa, Artificial sequence
  peptide
  MISC_FEATURE (1)..(13): X2, X4, and X6 are independently chosen as any amino acid with the proviso that at least one of X2, X4 and X6 is not S and at least one of X2, X4 and X6 is selected from Ala, Gly, Leu, Ile, Val, Pro or Met.
  MISC_FEATURE (7)..(7): Xaa=X2
  MISC_FEATURE (10)..(10): Xaa=X4
  MISC_FEATURE (12)..(12): Xaa=X6
KEQEMIXSVXQXE

SEQ ID NO: 56 - protein, 16 aa, Artificial sequence
  peptide
  MISC_FEATURE (1)..(16): X2, X4, and X6 are independently chosen as any amino acid with the proviso that at least one of X2, X4 and X6 is not S and at least one of X2, X4 and X6 is selected from Ala, Gly, Leu, Ile, Val, Pro or Met.
  MISC_FEATURE (2)..(2): Xaa=X2
  MISC_FEATURE (5)..(5): Xaa=X4
  MISC_FEATURE (7)..(7): Xaa=X6
TXPVXHXEDVQGAVQG

SEQ ID NO: 57 - protein, 16 aa, Artificial sequence
  peptide
  MISC_FEATURE (1)..(16): X2, X4, and X6 are independently chosen as any amino acid with the proviso that at least one of X2, X4 and X6 is not S and at least one of X2, X4 and X6 is selected from Ala, Gly, Leu, Ile, Val, Pro or Met.
  MISC_FEATURE (2)..(2): Xaa=X2
  MISC_FEATURE (5)..(5): Xaa=X4
  MISC_FEATURE (7)..(7): Xaa=X6
IXPVXHXESRPNVPQT

SEQ ID NO: 58 - protein, 16 aa, Artificial sequence
  peptide
  MISC_FEATURE (1)..(16): X2, X4, and X6 are independently chosen as any amino acid with the proviso that at least one of X2, X4 and X6 is not S and at least one of X2, X4 and X6 is selected from Ala, Gly, Leu, Ile, Val, Pro or Met.
  MISC_FEATURE (2)..(2): Xaa=X2
  MISC_FEATURE (5)..(5): Xaa=X4
  MISC_FEATURE (7)..(7): Xaa=X6
IXSVXQXENAPKITLS

SEQ ID NO: 59 - protein, 21 aa, Artificial sequence
  peptide
  MISC_FEATURE (1)..(21): X2, X4, and X6 are independently chosen as any amino acid with the proviso that at least one of X2, X4 and X6 is not S and at least one of X2, X4 and X6 is selected from Ala, Gly, Leu, Ile, Val, Pro or Met.
  MISC_FEATURE (7)..(7): Xaa=X2
  MISC_FEATURE (10)..(10): Xaa=X4
  MISC_FEATURE (12)..(12): Xaa=X6
KEQEMTXPVXHXEDVQGAVQG

SEQ ID NO: 60 - protein, 21 aa, Artificial sequence
  peptide
  MISC_FEATURE (1)..(21): X2, X4, and X6 are independently chosen as any amino acid with the proviso that at least one of X2, X4 and X6 is not S and at least one of X2, X4 and X6 is selected from Ala, Gly, Leu, Ile, Val, Pro or Met.
  MISC_FEATURE (7)..(7): Xaa=X2
  MISC_FEATURE (10)..(10): Xaa=X4
  MISC_FEATURE (12)..(12): Xaa=X6
KEQEMIXPVXHXESRPNVPQT

SEQ ID NO: 61 - protein, 21 aa, Artificial sequence
  peptide
  MISC_FEATURE (1)..(21): X2, X4, and X6 are independently chosen as any amino acid with the proviso that at least one of X2, X4 and X6 is not S and at least one of X2, X4 and X6 is selected from Ala, Gly, Leu, Ile, Val, Pro or Met.
  MISC_FEATURE (7)..(7): Xaa=X2
  MISC_FEATURE (10)..(10): Xaa=X4
  MISC_FEATURE (12)..(12): Xaa=X6
KEQEMIXSVXQXENAPKITLS

SEQ ID NO: 62 - protein, 8 aa, Artificial sequence
  peptide
TAPVAHAE

SEQ ID NO: 63 - protein, 13 aa, Artificial sequence
  peptide
KEQEMTAPVAHAE

SEQ ID NO: 64 - protein, 16 aa, Artificial sequence
  peptide
TAPVAHAEDVQGAVQG

SEQ ID NO: 65 - protein, 21 aa, Artificial sequence
  peptide
KEQEMTAPVAHAEDVQGAVQG

SEQ ID NO: 66 - protein, 32 aa, Artificial sequence
  peptide
YARAAARNARAEKEQEMTAPVAHAEDVQGAVQ

SEQ ID NO: 67 - protein, 8 aa, Artificial sequence
  peptide
IAPVAHAE

SEQ ID NO: 68 - protein, 13 aa, Artificial sequence
  peptide
KEQEMIAPVAHAE

SEQ ID NO: 69 - protein, 16 aa, Artificial sequence
  peptide
IAPVAHAESRPNVPQT

SEQ ID NO: 70 - protein, 21 aa, Artificial sequence
  peptide
KEQEMIAPVAHAESRPNVPQT

SEQ ID NO: 71 - protein, 8 aa, Artificial sequence
  peptide
IASVAQAE

SEQ ID NO: 72 - protein, 13 aa, Artificial sequence
  peptide
KEQEMIASVAQAE

SEQ ID NO: 73 - protein, 16 aa, Artificial sequence
  peptide
IASVAQAENAPKITLS

SEQ ID NO: 74 - protein, 21 aa, Artificial sequence
  peptide
KEQEMIASVAQAENAPKITLS

SEQ ID NO: 75 - protein, 12 aa, Artificial sequence
  peptide
  MISC_FEATURE (1)..(12): X1, X2, X3, X4, X5, X6 are independently chosen as defined with X2, X4, and X6 defined as any amino acid the proviso that at least one of X2, X4 and X6 is not S.
  MISC_FEATURE (1)..(1): X=X1=Thr or Ile
  MISC_FEATURE (2)..(2): X=X2
  MISC_FEATURE (3)..(3): X=X3=Pro or Ser
  MISC_FEATURE (5)..(5): X=X4
  MISC_FEATURE (6)..(6): X=X5=His or Gln
  MISC_FEATURE (7)..(7): X=X6
  MISC_FEATURE (9)..(9): Xaa=Arg or Tyr
  MISC_FEATURE (10)..(10): Xaa=Arg or Tyr
  MISC_FEATURE (11)..(11): Xaa=Arg or Tyr
  MISC_FEATURE (12)..(12): Xaa=Arg or Tyr
XXXVXXXEXXXX

(19) United States
(12) Patent Application Publication
(10) Pub. No.: US 2019/0345216 A1
(43) Pub. Date: Nov. 14, 2019

(54) SorCS PEPTIDES AND USES THEREOF

(71) Applicant: AARHUS UNIVERSITET, Aarhus C (DK)

(72) Inventors: Simon Molgaard Jensen, Hjortshøj (DK); Simon Glerup Pedersen, Risskov (DK)

(21) Appl. No.: 16/061,085

(22) PCT Filed: Dec. 19, 2016
(86) PCT No.: PCT/DK2016/050445

§ 371 Date: Jun. 11, 2018

(30) Foreign Application Priority Data
Dec. 18, 2015 (DK) PA 2015 70835

(51) Int. Cl. C07K 14/705

(52) U.S. Cl. CPC C07K 14/705; A61K 38/00

(57) ABSTRACT

The present invention concerns compounds and methods for modulating the phosphorylation of the Vps10 domain-containing receptor SorCS2, SorCS1 or SorCS3, and/or expression thereof. By said modulation the invention is useful for treatment and amelioration of neurological, mental and behavioural, metabolic, eating, and neoplastic disorders.

### Field of invention

The present invention concerns peptides derived from the C-terminal cytoplasmic domain of the Vps10p domain receptors SorCS1, SorCS2 and SorCS3, and medical uses thereof, such as treatment of neoplastic disorders and disorders of the nervous system.

### BACKGROUND OF INVENTION

Synaptic plasticity is the ability of a synapse to change in strength and is considered as the cellular basis of most cognitive processes, including memory formation. Strengthening or weakening of synapses occurs via long-term potentiation (LTP) and long-term depression (LTD), respectively. The molecular mechanisms underlying synaptic plasticity are incompletely understood. BDNF/proBDNF is a plasticity-related protein that is released into the pre-synaptic space as a result of neuronal activity, and the subsequent interaction of BDNF with the postsynaptic tyrosine kinase receptor TrkB is required for the induction of the early-phase of LTP (E-LTP) (23). Neuronal activity also recruits TrkB into postsynaptic densities (PSD), thereby allowing BDNF signalling and consequent late-phase LTP at stimulated synapses (20-22). BDNF also plays an important role in synapse formation and dendritic complexity in both adult and developing neurons. BDNF therefore plays a central role in activity-dependent formation of neuronal connectivity.

The receptor SorCS2 has recently been identified as a p75^{NTR} co-receptor required for binding of proBDNF (12,13). SorCS2 belongs to the Vps10p-domain/sortilin family of sorting and signaling receptors, a protein family that also includes sortilin, SorLA, and SorCS-1 and -3 (14, 15). All five receptors are highly expressed in both the developing and adult nervous system. They share a characteristic N-terminal Vps10p-domain that has high homology to Vps10p, a vacuolar protein-sorting protein in yeast. In fact, sortilins are capable of golgi-endosome trafficking, internalisation, and polarized anterograde transport, suggesting key roles in regulating neuronal function (16-18).

Interestingly, BDNF may play a role in preventing formation of amyloid plaques, considered to be the pathological hallmark of Alzheimer's disease (5) and BDNF upregulation confers protection against oligomeric and/or fibrillar A1-42-induced cell death (6). Furthermore, loss of cortically supplied BDNF leads to striatal degeneration and choreic movements that characterize Huntington's disease patients (4). BDNF has also been associated with diabetes and obesity, deletion of BDNF in the postnatal brain leading to obesity in mice (8) and BDNF regulating eating behaviour in mice (9). BDNF also prevents the development of diabetes in pre-diabetic mice (10), and has been shown to regulate glucose metabolism by modulating energy balance in diabetic mice (11). Furthermore obesity in the WAGR syndrome is attributable to deletions that induce haploinsufficiency of BDNF (7).

Recently, Glerup et al. showed that SorCS2 plays a critical role in mediating BDNF response. Therein, neurons lacking SorCS2 failed to respond to BDNF and elicit LTP. Furthermore, neurons lacking SorCS2 also failed to increase in dendritic complexity and spine density upon stimulation with BDNF, indicating a critical role of SorCS2 in mediating the BDNF signaling (2).

Due to the increasing occurrence of the above-mentioned conditions in the developed world, there is a need for compounds that can be used to treat these diseases. If an underlying genetic pleiotropy exists, it would be advantageous to exploit such a situation by targeting members of the common signalling pathways.

### SUMMARY OF INVENTION

The present inventors have surprisingly found that phosphorylation of the intracellular C-terminal domain of the Vps10p-domain receptors SorCS2, SorCS1 and SorCS3 results in restoration of impaired synaptic plasticity. Based on this finding, the inventors have developed peptides and peptide analogues capable of mimicking the endogenous phosphorylation of said intracellular domains, and demonstrated that said peptides are capable of restoring impaired synaptic plasticity, morphological plasticity and/or neuronal plasticity. They have also prepared peptides capable of inhibiting the very same neurons thereby offering a remedy for neurological disorders, such as epilepsy, Huntington's disease (HD), and Alzheimer's disease (AD), mental or bahavioural disorders such as depression, anxiety, obsessive compulsive disorder (OCD), bipolar disorder (BD), Schizophrenia (SZ), and pervasive developmental disorders, while also offering a treatment for WAGR-related Wilm's kidney tumours, obesity, insulin resistance and diabetes.

Accordingly, in one aspect the invention concerns a polypeptide comprising the C-terminal cytoplasmic domain of a Vps10p-domain receptor selected from the group consisting of SorCS2, SorCS1, and SorCS3, wherein at least one amino acid of said C-terminal cytoplasmic domain has been altered to a different amino acid residue.

Therefore, in one aspect the invention concerns a peptide or peptide analogue (P₁), comprising or consisting of the sequence X₁X₂X₃VX₄X₅X₆E (SEQ ID NO: 9), wherein
(i) X₁ is threonine (T) or isoleucine (I)
(ii) X₂ is selected from the group consisting of phosphoserine (J), aspartic acid (D), glutamic acid (E), alanine (A) and serine (S)
(iii) X₃ is proline (P) or serine (S)
(iv) X₄ is selected from the group consisting of phosphoserine (J), aspartic acid (D), glutamic acid (E), alanine (A) and serine (S).
(v) X₅ is histidine (H) or glutamine (Q), and
(vi) X₆ is selected from the group consisting of phosphoserine (J), aspartic acid (D), glutamic acid (E), alanine (A) and serine (S).

In one aspect the invention concerns a peptide or peptide analogue as described herein for use as a medicament.

In one aspect the invention concerns a polynucleotide encoding a peptide as defined herein.

In one aspect the invention concerns a vector comprising a polynucleotide encoding a peptide as described herein.

In one aspect the invention concerns a host cell, such as a bacterial host cell, a mammalian host cell, such as a human host cell, comprising a polynucleotide or a vector as described herein.

In another aspect the invention concerns a composition, preferably a pharmaceutically acceptable composition, comprising any one or more of a peptide or peptide analogue, a polynucleotide, a vector, or a host cell as described herein.

In one aspect the invention concerns a peptide or peptide analogue as described herein, a composition, a polynucleotide, a vector, or a host cell as described herein for use in the manufacture of a medicament, for prophylaxis and/or treatment of a disorder selected from the group consisting of neoplasia and diseases of the nervous system.

In one aspect the present invention concerns a method for treatment or prophylaxis of neoplastic disorders or a disease of the nervous system, said method comprising the administration of a peptide or peptide analogue as described herein, a composition, a polynucleotide, a vector, or a host cell as described herein to a subject in need thereof.

In one aspect the present invention concerns a method for identifying a compound capable of modulating phosphorylation of the C-terminal cytoplasmic domain of a Vps10p-domain receptor selected from the group consisting of SorCS2, SorCS1, SorCS3, the method comprising the steps of:
(i) providing one or more candidate compounds and a cell expressing SorCS2, SorCS1, SorCS3;
(ii) measuring the phosphorylation level of the protein of (i) in said cell in the absence of said candidate compound;
(iii) contacting the cell of (ii) with said candidate compound;
(iv) measuring the phosphorylation level of the protein of (iii);
(v) comparing the phosphorylation levels of (ii) and (iv),
(vi) identifying the compound by electing compounds of (v) which are capable of modulating phosphorylation of the C-terminal domain of SorCS2, SorCS1, or SorCS3.

In one aspect the present invention concerns a method for identifying a compound capable of modulating expression of SorCS2, SorCS1, and/or SorCS3, the method comprising the steps of:
(i) providing one or more candidate compounds and a cell expressing SorCS2, SorCS1 and/or SorCS3;
(ii) measuring the expression level of the protein from (i) in said cell in the absence of said candidate compound;
(iii) contacting the cell of (ii) with said candidate compound;
(iv) measuring the expression level of the protein of (iii);
(v) comparing the expression levels of (ii) and (iv),
(vi) identifying the compound by electing compounds of (v) which are capable of modulating expression of SorCS2, SorCS1 and/or SorCS3.

In one aspect the present invention concerns a method for increasing the number of synapses, said method comprising the administration of a peptide or peptide analogue as described herein, or a composition, or the polynucleotide, or a vector, or a host cell as described herein, to a subject in need thereof.

In one aspect the present invention concerns a method for promoting changes in neuronal morphology, said method comprising the administration of a peptide or peptide analogue as described herein, or a composition, or the polynucleotide, or a vector, or a host cell as described herein, to a subject in need thereof.

In another aspect the present invention concerns a method for inducing phosphorylation of the C-terminal cytoplasmic domain of the Vps10p-domain receptors SorCS2, SorCS1 and/or SorCS3 as described herein, in primary hippocampal neurons, said method comprising the administration of a peptide or peptide analogue as described herein, or a composition, or the polynucleotide, or a vector, or a host cell as described herein, to a subject in need thereof.

### DESCRIPTION OF DRAWINGS

FIG. 1: SorCS2 is required for fluoxetine treatment response in mice. Wildtype (wt) and SorCS2^{-/-} mice were tested in the marble burying test which is a test used to evaluated anxiety, depression, obsessive compulsive disorder among other things. Here, SorCS2^{-/-} mice buried similar levels of marbles as wt mice. However, while wildtype mice were calmed and buried less marbles upon injection of fluoxetine, this was not the case for SorCS2^{-/-} mice.

FIG. 2: SorCS2 is important for BDNF-mediated increase in the number of synapses.

An increase in the number of inhibitory synapses on wt neurons is seen upon stimulation with BDNF (A). This is not the case for neurons lacking SorCS2 (B). Similarly, an increase in the number of glutamatergic synapses is seen in wt neurons upon stimulation with BDNF (C) while this is not the case for neurons lacking SorCS2 (D).

FIG. 3: SorCS2 is required for dendritic branching.

Lack of SorCS2 affects dendritic branching. Upon stimulation with BDNF, glutamatergic neurons increase in complexity (A), while this is not the case for neurons lacking SorCS2 (B). Similarly, wild-type GABAergic interneurons also increase in complexity upon stimulation with BDNF while interneurons lacking SorCS2 are non-responsive to BDNF (C and D).

FIG. 4: SorCS2 is important for mediating BDNF signalling.

(A) IP of SorCS2 from neurons pre-incubated with radioactive phosphate showed increased phosphorylation upon BDNF-stimulation (left) compared to unstimulated (right). (B) Transfection of SorCS2^{-/-} neurons with a full length SorCS2 1) rescues the response to BDNF. However, upon transfection with a variant lacking the intracellular domain, no increase in neurite branching is seen upon BDNF stimulation 2). Creating variants with a deletion of the last 21 3) or 35 4) amino acids still rescued the phenotype upon stimulation with BDNF. However, upon deletion of 56 amino acids 5) the response to BDNF was lost.

FIG. 5: Multiple serines on the C-terminal domain of SorCS2 are required for BDNF-mediated response.

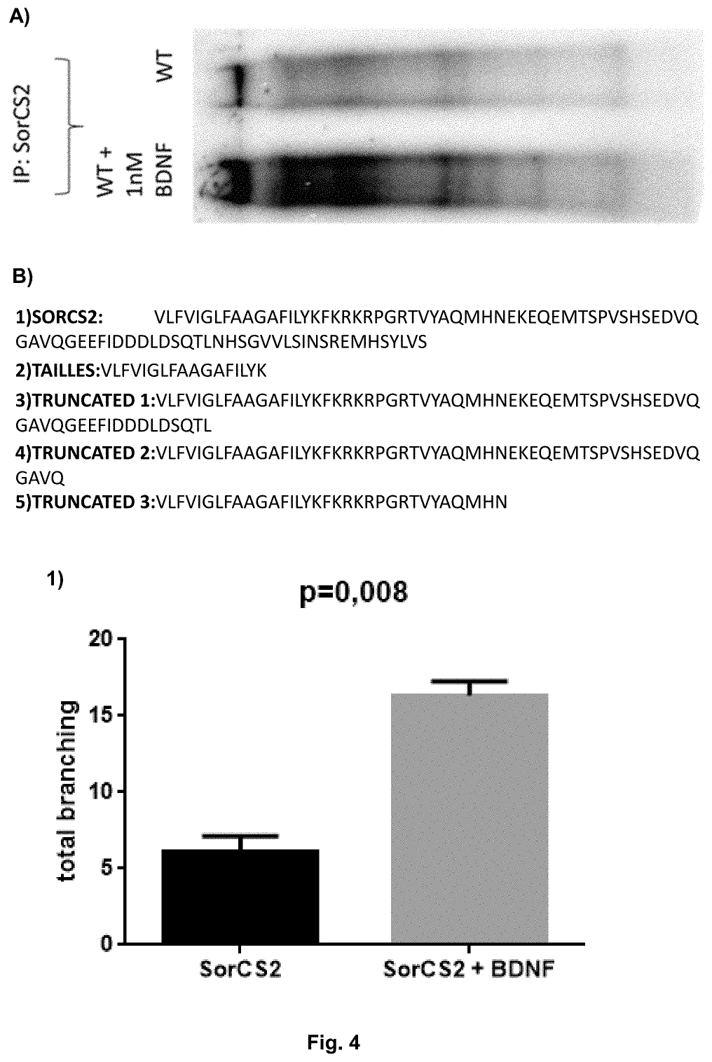

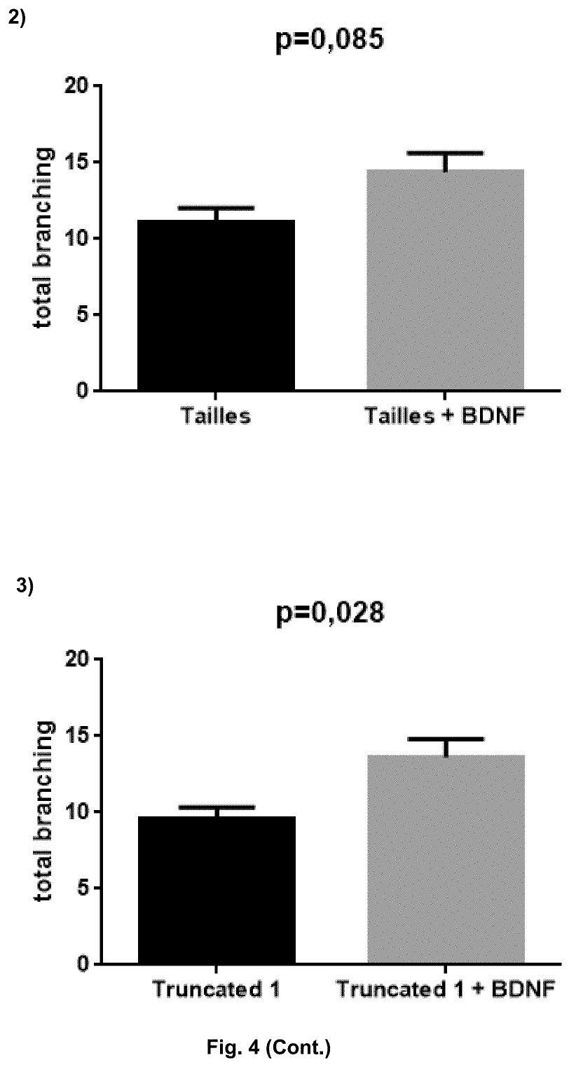

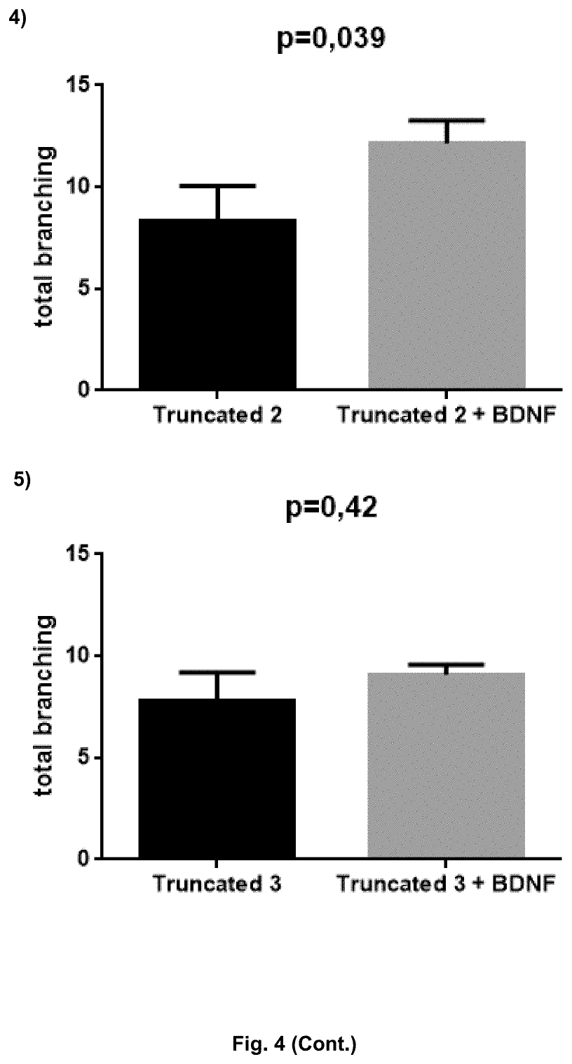

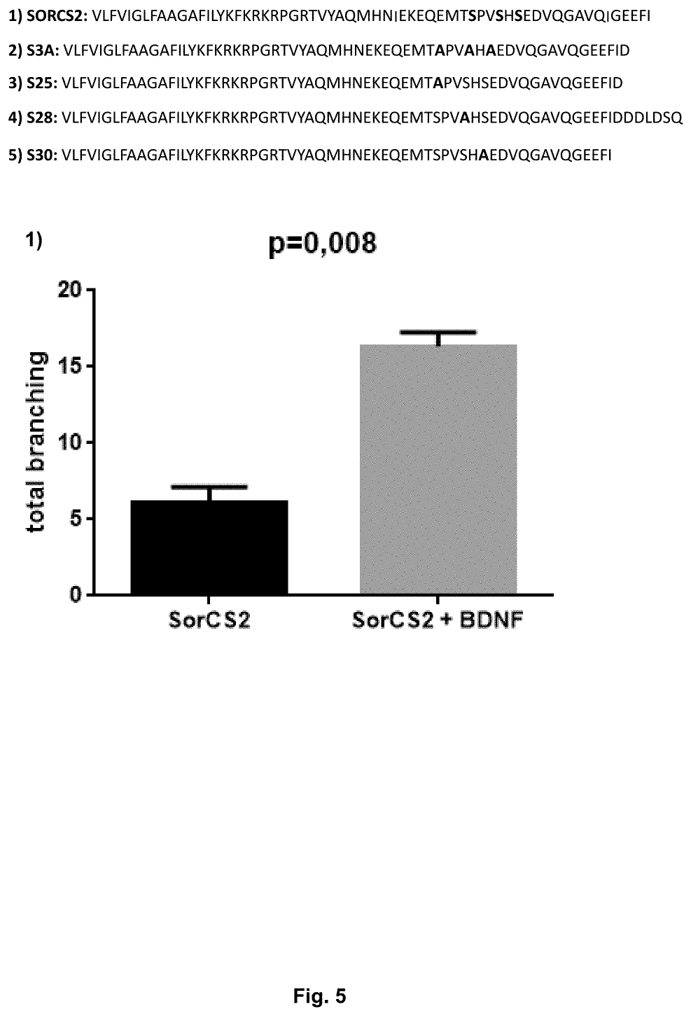

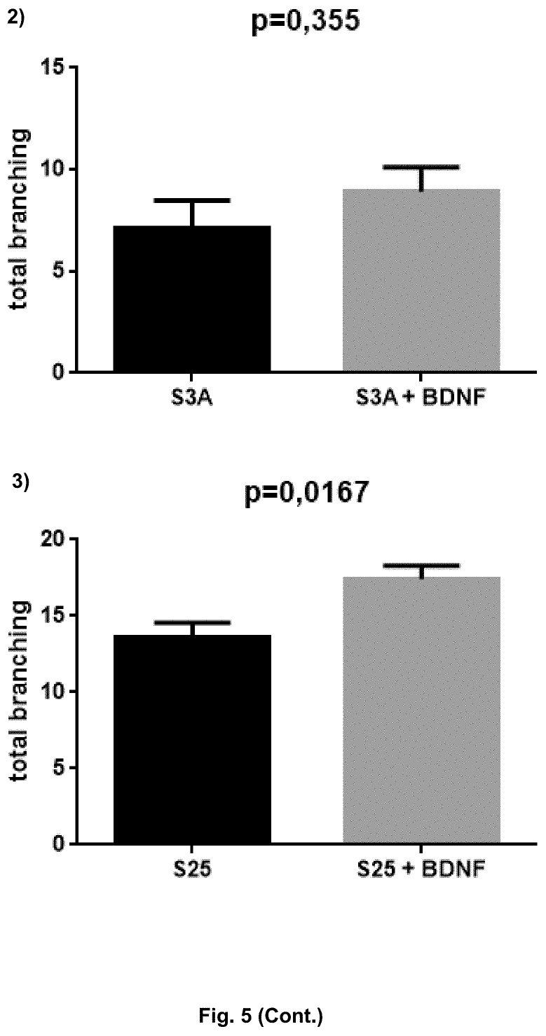

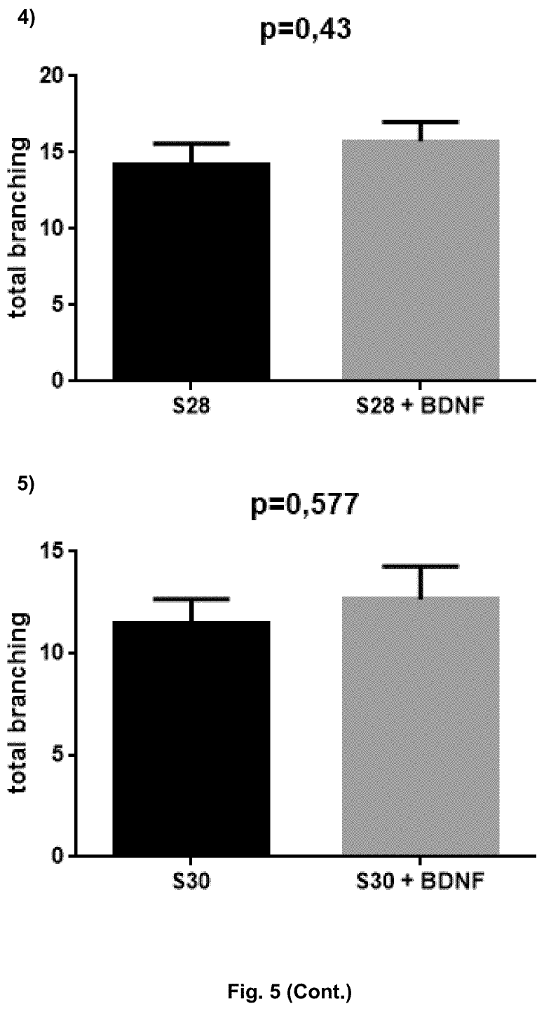

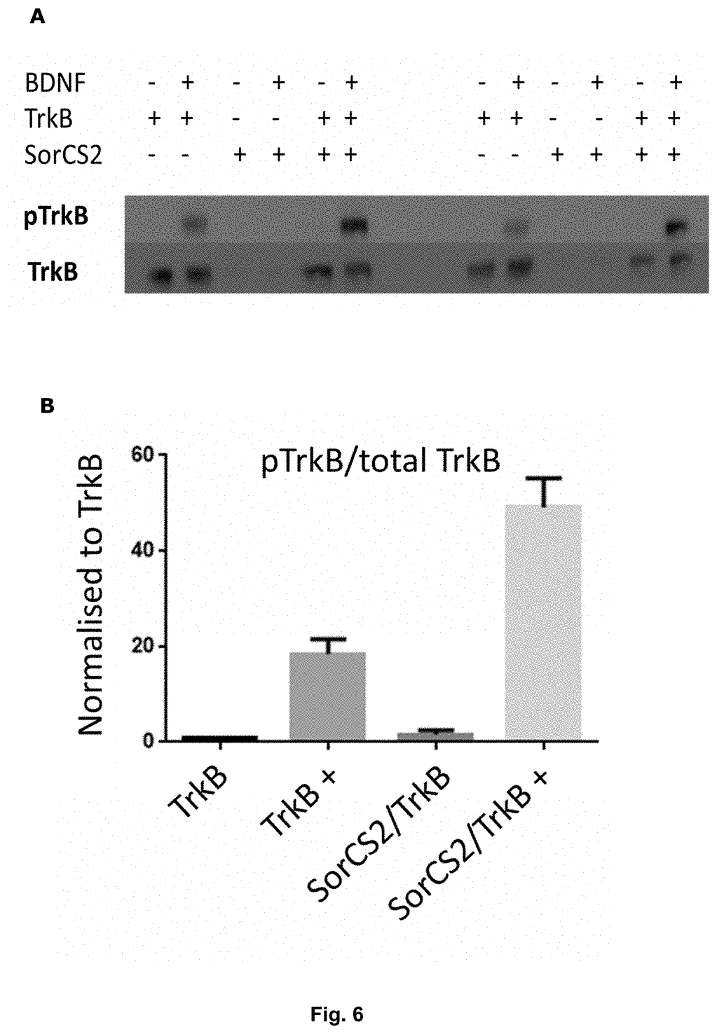

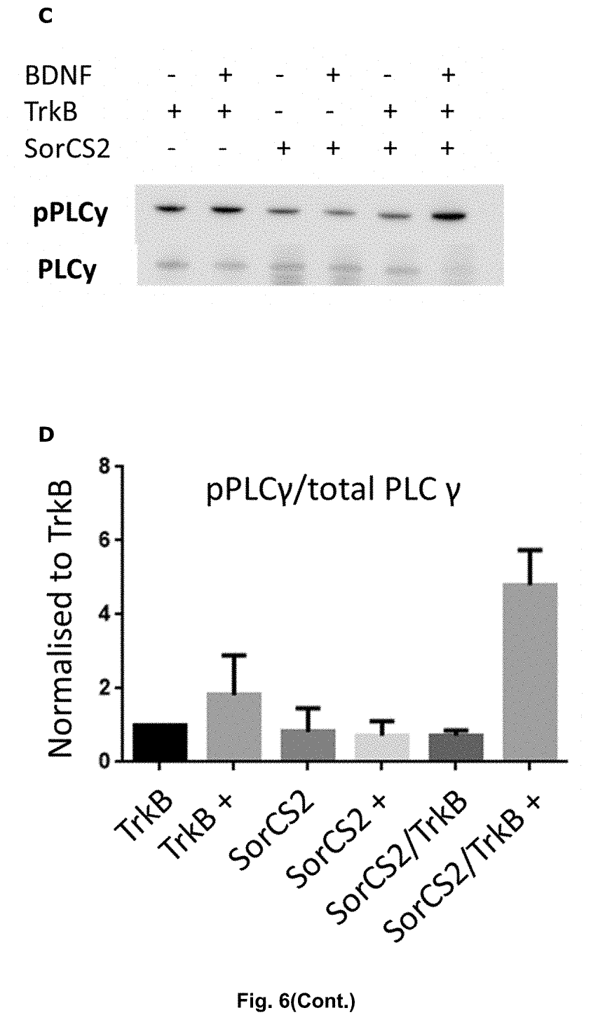

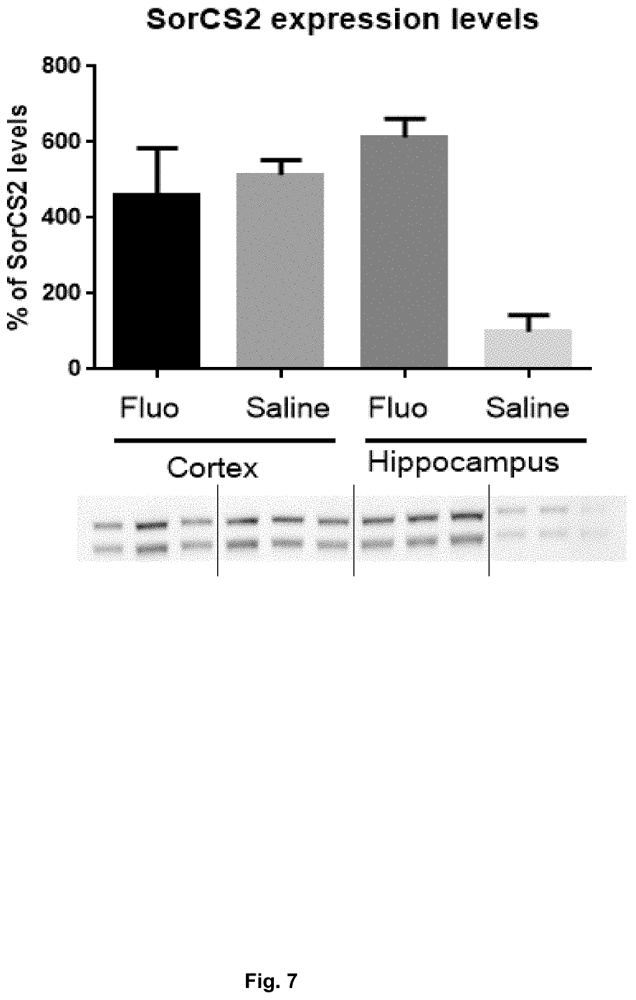

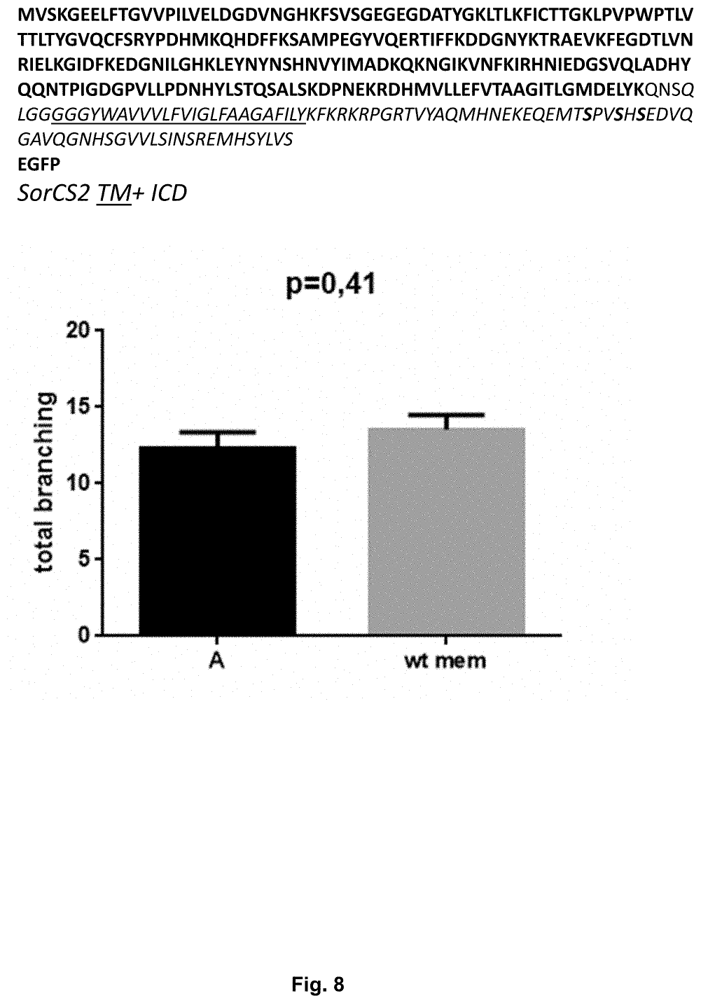

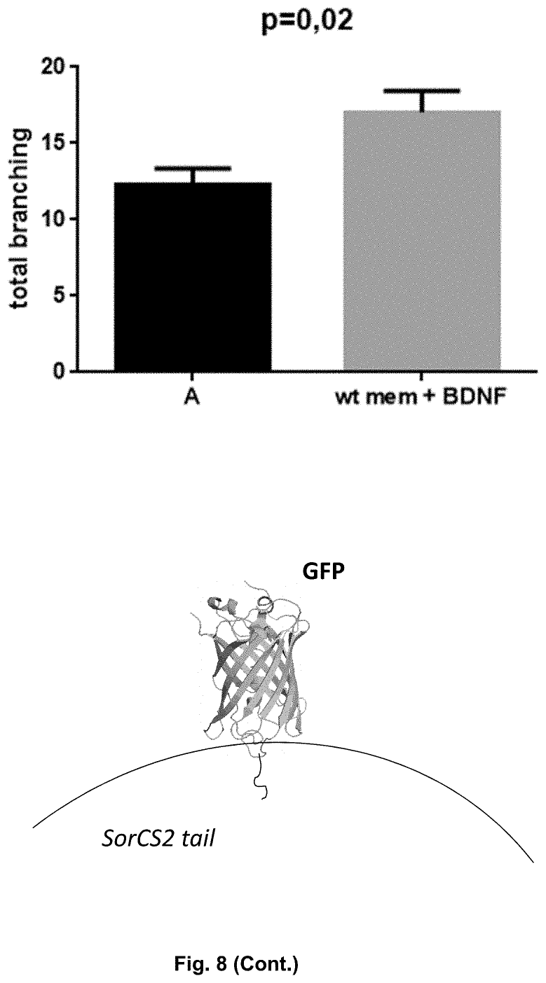

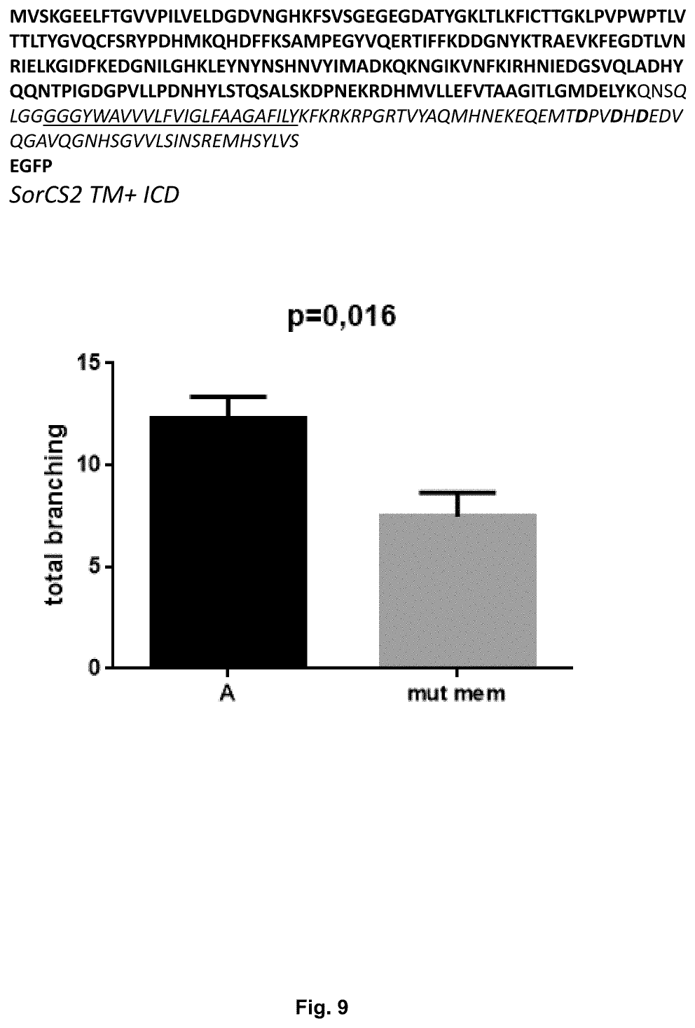

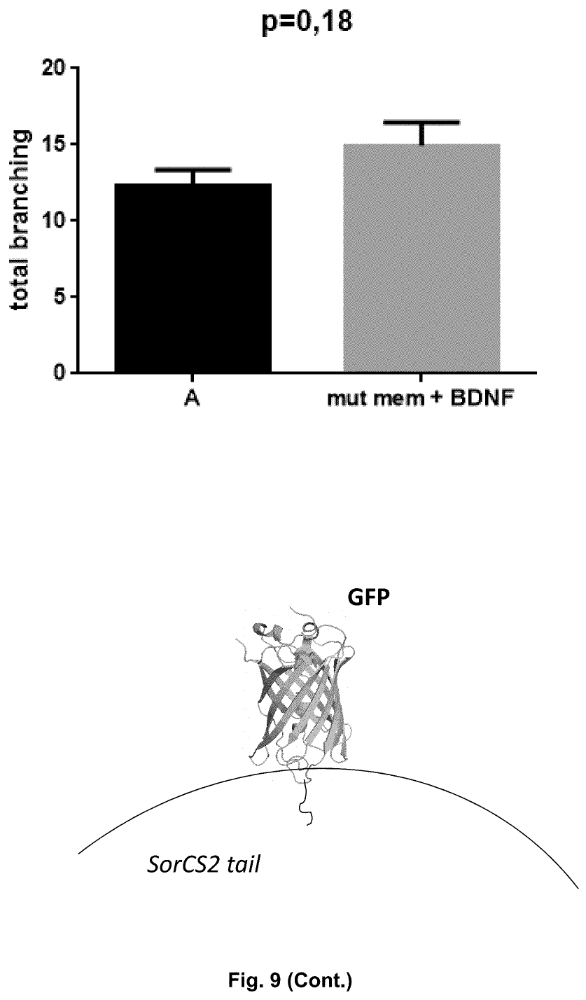

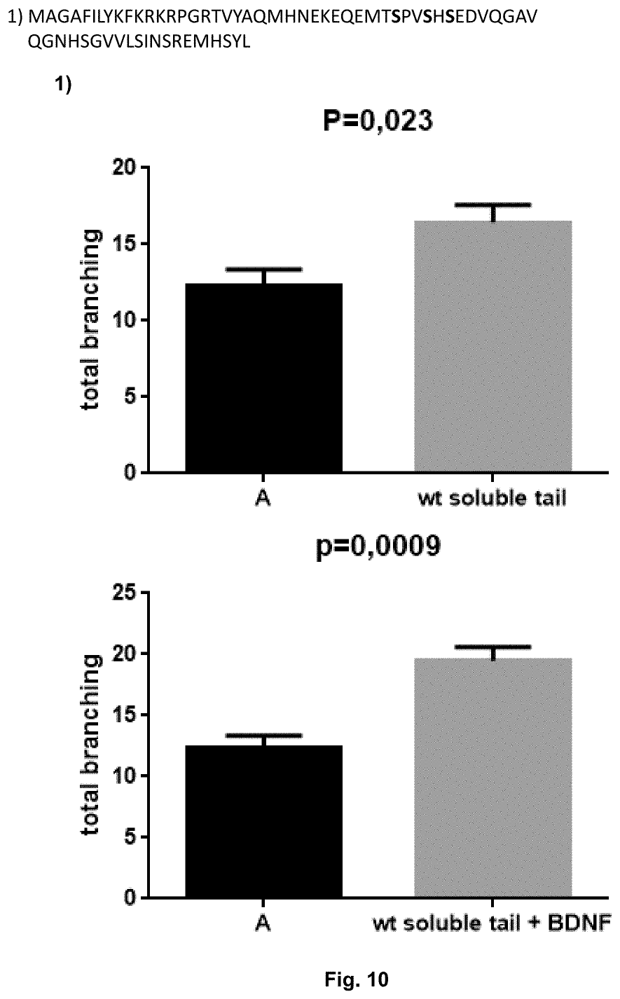

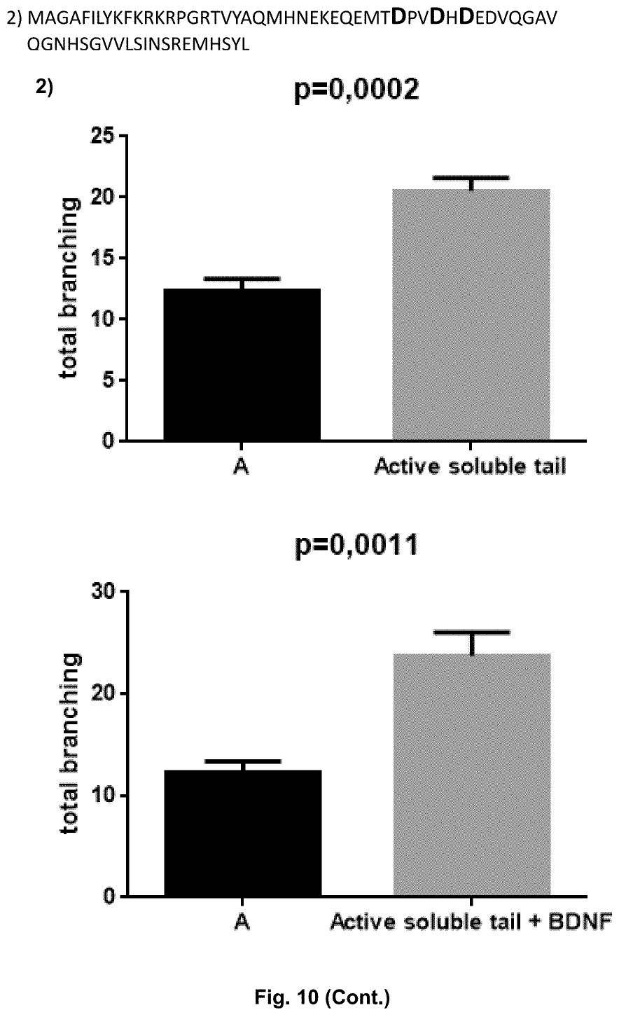

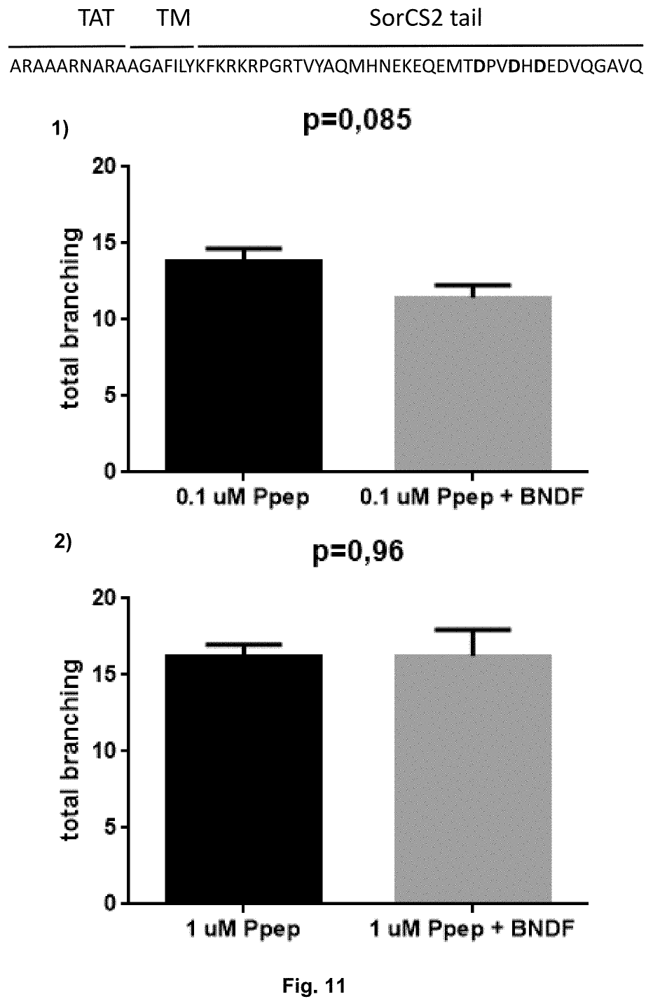

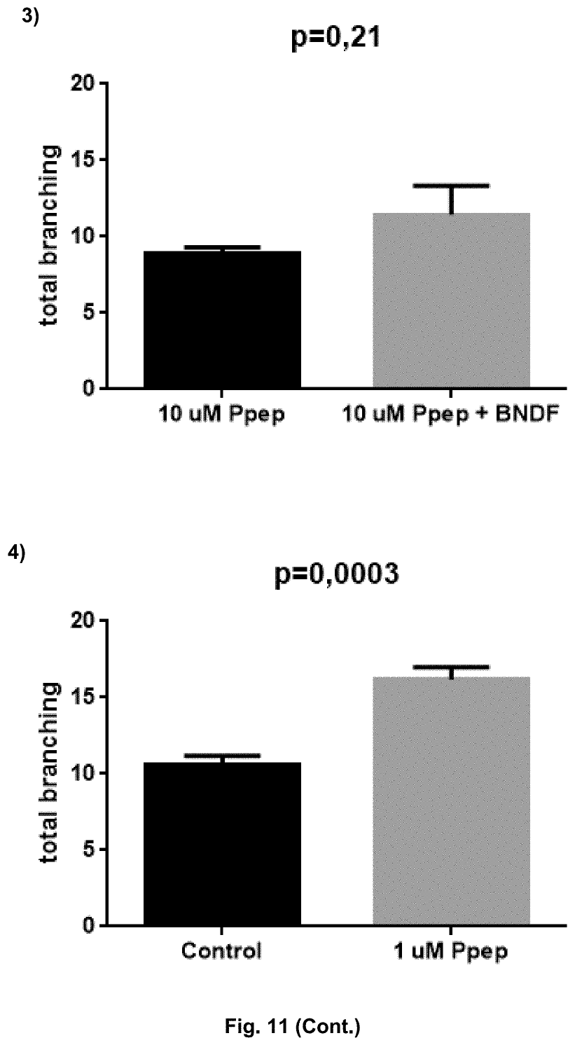

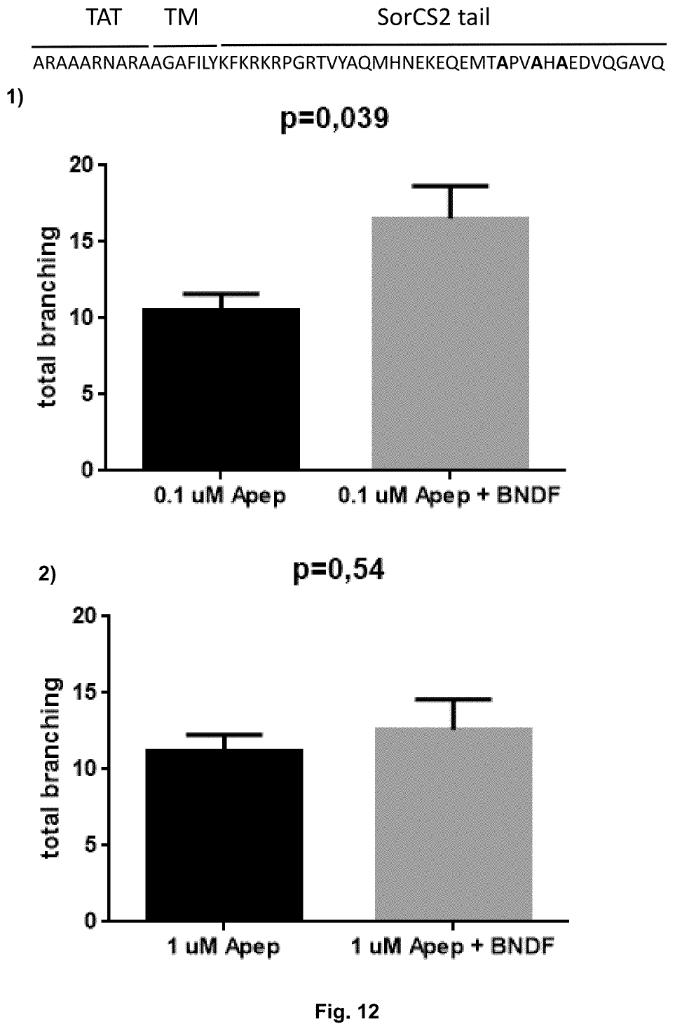

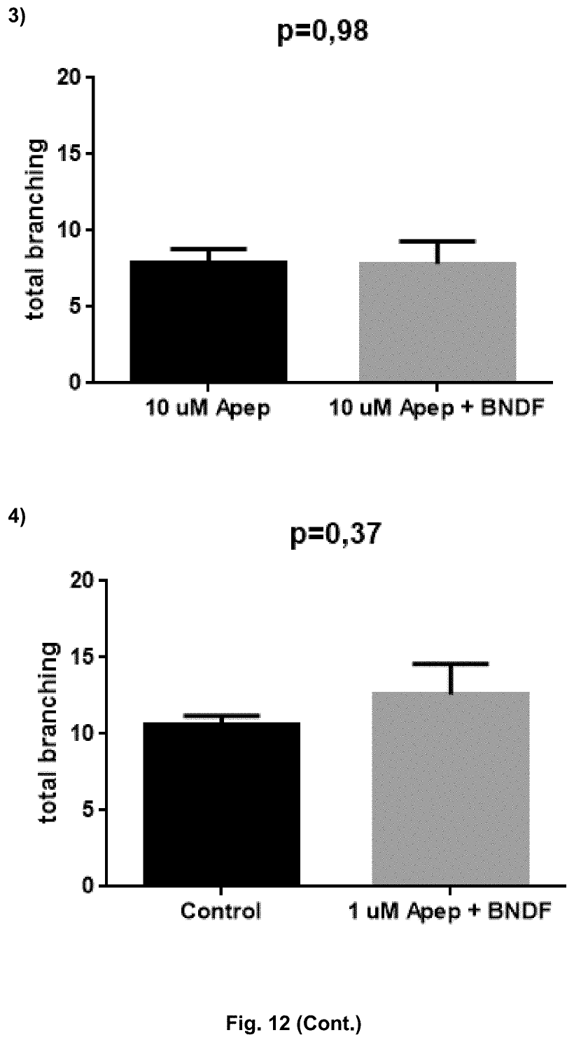

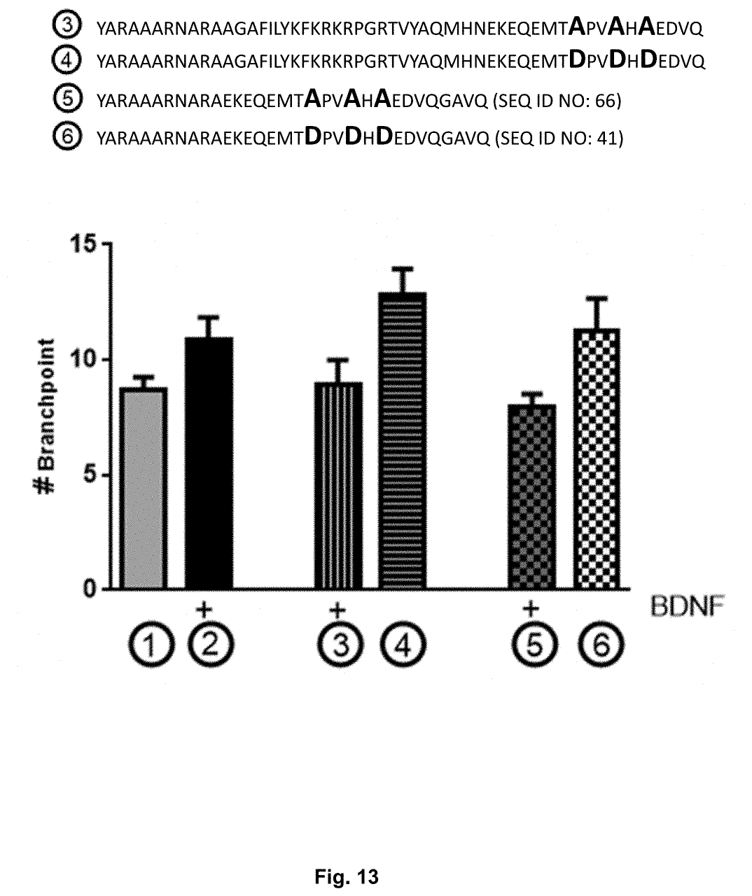

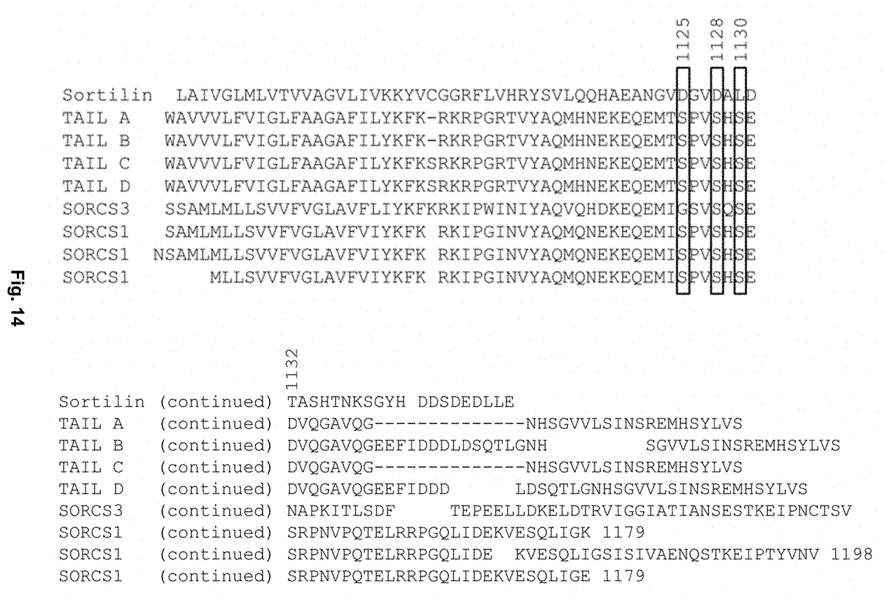

1) Transfection of full length SorCS2 is able to rescue response to BDNF in neurons lacking SorCS2. Here, the sequence which was shown in FIG. 4 to be important to the response to BDNF is highlighted. Serines at position 1125, 1128 and 1130 are highlighted. By creating a full-length variant where serines on 3 different positions had been changed to alanines (S1125A, S1128A and S1130A), the response was diminished 2), indicating that these 3 serines are critical for mediating BDNF stimulus. Constructs were made where any of the 3 serines were mutated to alanines: S1125A 3)(denoted “S25” in Figure), S1128A 4) (denoted “S28” in Figure) and S1130A 5) (denoted “S30” in Figure). When mutating serines at position 1128 and 1130, but not 1125, the response to BDNF was lost, indicating an important role of the serines at positions 1128 and 1130 in mediating the response to BDNF.

FIG. 6: SorCS2 is important for phosphorylation of the BDNF-receptor TrkB. hSY5Y cells were transfected with either SorCS2, TrkB or both (panels A and B). Upon stimulation with BDNF a 2.5 fold increase in phosphorylation of TrkB was seen when SorCS2 was also present, compared to when only TrkB was present (panel B). Furthermore, a significant increase was also seen in phosphorylation of PLCy, a downstream kinase, when SorCS2 was present compared to when only TrkB was transfected (panels C and D). This indicates a critical role of SorCS2 in mediating the response to BDNF.

FIG. 7: Fluoxetine affects SorCS2 expression levels in the hippocampus. Wt mice were given either normal water or water containing 0.08 mg/ml fluoxetine for 3 weeks. After 3 weeks the mice were euthanized by cervical dislocation and hippocampus and cortex was dissected out. Western blot analysis revealed an increase in expression of SorCS2 in the hippocampus but not in the cortex. The samples have been normalised to hippocampus without fluoxetine treatment.

FIG. 8: The extracellular domain is of SorCS2 is not required for neurotrophic response.

To evaluate if the extracellular domain plays a role in the response to BDNF, constructs were made where the extracellular part of SorcS2 have been changed to GFP to stabilise insertion into the plasmamembrane (denoted “wt mem” in the Figure). When compared to SorCS2^{-/-} neurons transfected with full length SorCS2 (denoted “A” in the Figure), no difference was seen. However, when stimulating wt mem with BDNF, a significant increase in morphological response was seen, indicating that the extracellular domain is not critical for the function of SorCS2 in mediating the response to BDNF.

FIG. 9: Activation of SorCS2 at the plasmamembrane does not confer increased morphological response.

To evaluate if activated SorCS2 bound the membrane would induce neurotrophic signalling, a construct was created where, similar to in FIG. 8, the extracellular domain of SorcS2 was substituted to GFP to stabilise insertion into the plasmamembrane. Furthermore, the 3 serines at position 1125, 1128 and 1130, in the intracellular tail of SorCS2, has been changed to aspartic acids to mimic phosphorylation of the serines (denoted mut mem in the Figure). A significant reduction in morphology was observed in mut mem when compared to SorCS2^{-/-} neurons transfected with full length SorCS2 (denoted A in the Figure). Upon addition of BDNF, the difference is rescued and no difference is observed between A and mut mem. This indicates that activation of SorCS2 at the plasmamembrane does not confer increased morphological response.

FIG. 10: Activation of the SorCS2 tail is sufficient to induce a response similar to the addition of BDNF.

To evaluate if a soluble variant of the SorCS2 tail was sufficient to induce a response to BDNF, a construct was created with the SorCS2 tail, without the extracellular or transmembrane domain (1, denoted “wt soluble tail” in the Figure). Here, transfection with wt soluble tail into SorCS2^{-/-} neurons created a significant increased response to BDNF when compared to SorCS2^{-/-} neurons transfected with full length SorCS2 (denoted “A” in the Figure). This response was further increased when stimulating wt soluble tail with BDNF. We therefore created a construct where serines at position 1125, 1128 and 1130 were changed to aspartic acids to mimic a phosphorylated serine (2, denoted “Active soluble tail” in the Figure). Here, transfection with Active soluble tail into SorCS2^{-/-} neurons created a significant increase in morphological complexity when compared to SorCS2^{-/-} neurons transfected with full length SorCS2 (denoted “A” in the Figure). Addition of BDNF did not further increase the response. Furthermore, the response was similar to wt soluble tail+BDNF, indicating that the SorCS2 tail is not only critical for the response to BDNF, activation of the tail is sufficient to induce a response similar to addition of BDNF.

FIG. 11: Constitutive active SorCS2 tail induces a response which is not further increased upon addition of BDNF.

Since transfection using an active soluble variant of the SorCS2 tail was sufficient to induce a response to BDNF (FIG. 10), we created peptides where serines at position 1125, 1128 and 1130 of the SorCS2 tail were changed to aspartic acids to mimic a constitutive active SorCS2 tail (Denoted “pPep” in Figure). Furthermore, the peptide constitutes a TAT-sequence (a HIV protein transduction domain) to facilitate entry into the cell. No difference in morphological response was observed between pPep and pPep+BDNF at neither 0.1 (1), 1 (2) or 10 (3) μM of pPep. However, when compared to a control (Where no BDNF has been added) (4) a significant response was observed when applying 1 μM pPep. This indicates that applying 1 μM of pPep induces a response which is not further increased upon addition of BDNF.

FIG. 12: Constitutively inactive SorCS2 tail is able to inhibit BDNF induced morphological response, even in the presence of BDNF.

Since addition of pPep was sufficient to induce a neurotrophic response, even in the absence of BDNF, we wanted to investigate if addition of an inactive peptide was sufficient to inhibit BDNF signalling, even in the presence of BDNF. We therefore created a peptide where serines at positions 1125, 1128 and 1130 of the SorCS2 tail had been changed to alanines to mimic an inactive SorCS2 tail (denoted Apep in Figure). Furthermore, the peptide constitutes a TAT-sequence (a HIV protein transduction domain) to facilitate entry into the cell. At 0.1 μM of Apep BDNF was still able to induce a significant response (1). However, this was not the case when applying 1 μM Apep (2) or 10 μM (3). When compared to control, no difference was observed between a control (where no BDNF had been added) and 1 μM Apep+BDNF (4). This indicates that at 1 μM, Apep is able to inhibit BDNF induced morphological response, even in the presence of BDNF.

FIG. 13: Short peptides are able to elicit both activation and inhibition of neurotrophic response.

Smaller peptides were created to study if these would be sufficient to induce a neurotrophic response. When adding 1 nM BDNF (2), wt neurons increased in complexity compared to WT neurons without BDNF (1). As seen before, a peptide where serines at position 25, 28 and 30 had been mutated to alanines inhibits morphological response, even in the presence of BDNF (3), and a peptide where serines at position 25, 28 and 30 had been changed to aspartic acids was able to induce a neurotrophic response, even in the absence of BDNF (4). We therefore created smaller peptides where serines had been changed to alanines that was still able to inhibit the neurotrophic response induced by BDNF (5). Furthermore, a short peptide where serines had been changed to aspartic acids was able to induce a neurotrophic response, even in the absence of BDNF (6)

FIG. 14: SorCS family tail sequence alignment.

Alignment of the tails from SorCS1, SorCS2, SorCS3 and sortilin shows highly conserved tails between SorCS1, SorCS2 and SorCS3, but not sortilin. Furthermore, in SorCS1, Serines at positions 1125, 1128 and 1130 are conserved. In SorCS3, serines at positions 1128 and 1130, (the serines found to be important to BDNF response in SorCS2, FIG. 5), are conserved. This indicates a possible similar role of SorCS1 and SorCS3 in regulating neurotrophic response.

### DETAILED DESCRIPTION OF THE INVENTION

Definition of Abbreviations and Terms

Cell penetrating peptide (CPP): the term refers to a peptide characterized by the ability to cross the plasma membrane of mammalian cells, and thereby may give rise to the intracellular delivery of cargo molecules, such as peptides, proteins, oligonucleotides to which it is linked.

Tat peptide: The TAT peptide of sequence GRKKRRQRRRPQ is derived from the transactivator of transcription (TAT) of human immunodeficiency virus and is a Cell-penetrating peptides.

Fluorophore: this term as understood herein is used interchangeably with the term “fluorescent moiety” and refers to any substance that can re-emit light upon excitation. Fluorescence is generated when the fluorophore, lying in its ground state, absorbs light energy at a short wavelength, creating an excited electronic singlet state, and emits light energy at a longer wavelength, creating a relaxed singlet state. The fluorophore then returns to its ground state.

Modulation: the term refers to a positive modulation, i.e. an induction, or a negative modulation, i.e. a reduction or inhibition. In the present application, a compound capable of modulating phosphorylation of the C-terminal domain of the Vps10 domain-containing receptor SorCS2 shall also designate compounds capable of mimicking the Vps10 domain-containing receptor SorCS2, wherein the C-terminal domain is phosphorylated.

Peptide analogue: The term “peptide analogue” as used herein refers to a compound comprising a peptide, wherein the peptide may be modified with moieties that do not necessarily consist of amino acid residues. A fluorescently tagged peptide for example is a peptide analogue.

### I. Synaptic Plasticity

Synaptic plasticity as referred to herein is the ability of a synapse to change in strength and is considered as the cellular basis of most cognitive processes, including memory formation. Strengthening or weakening of synapses occurs via long-term potentiation (LTP) and long-term depression (LTD), respectively. Neuronal activity induces presynaptic release of brain-derived neurotrophic factor BDNF, and the subsequent interaction of BDNF with the postsynaptic tyrosine kinase receptor TrkB is required for the induction of the early-phase of LTP (E-LTP) (23). In addition, the release of the precursor of BDNF, proBDNF, and the subsequent conversion of proBDNF into mature BDNF by extracellular proteinases, is required for the late-phase of LTP and synapse consolidation (25). In contrast, weakening of synapses is facilitated by a different mechanism, involving the interaction of proBDNF with the postsynaptic pan-neurotrophin receptor p75 (p75^{NTR}), which results in LTD (24). Dendritic growth is regulated locally by synaptic activity as well as molecular signals from neighbouring cells. These activity-dependent structural changes act, together with efficacy of local synapses, to shape specific patterns of connectivity in the nervous system.

### II. Peptide or Peptide Analogue

The invention is as defined in the claims.

In one aspect the invention concerns a polypeptide comprising the C-terminal cytoplasmic domain of a Vps10p-domain receptor selected from the group consisting of SorCS2, SorCS1, SorCS3, wherein at least one amino acid of said C-terminal cytoplasmic domain has been altered to a different amino acid residue.

Therefore, in one aspect the invention concerns a peptide or peptide analogue (P₁), comprising or consisting of the sequence X₁X₂X₃VX₄X₅X₆E (SEQ ID NO: 9), wherein
(i) X₁ is selected from threonine (T), isoleucine (I)
(ii) X₃ is selected from proline (P), serine (S)
(iii) X₅ is selected from histidine (H), glutamine (Q), and
(iv) at least one of of X₂, X₄, X₆ is selected from the group consisting of phosphoserine (J), aspartic acid (D), glutamic acid (E), alanine (A).

In a preferred embodiment, X₁ is threonine (T), X₃ is proline (P), and X₅ is histidine (H), and P₁ is thus SEQ ID NO: 13.

In some embodiments, the present invention relates to a peptide or peptide analogue (P₁) comprising or consisting of the sequence KEQEMX₁X₂X₃VX₄X₅X₆E (SEQ ID NO: 10).

In a preferred embodiment, X₁ is threonine (T), X₃ is proline (P), and X₅ is histidine (H), and P₁ is thus SEQ ID NO: 16

In other embodiments, the present invention relates to a peptide or peptide analogue (P₁) comprising or consisting of the sequence X₁X₂X₃VX₄X₅X₆EX₇X₈X₉X₁₀X₁₁X₁₂X₁₃X₁₄ (SEQ ID NO: 11), wherein
(i) X₇ is selected from aspartic acid (D), asparagine (N), serine (S)
(ii) X₈ is selected from valine (V), arginine (R), alanine (A)
(iii) X₉ is selected from proline (P), glutamine (Q)
(iv) X₁₀ is selected from glycine (G), lysine (K), asparagine (N)
(v) X₁₁ is selected from alanine (A), isoleucine (I), valine (V)
(vi) X₁₂ is selected from valine (V), threonine (T), proline (P)
(vii) X₁₃ is selected from glutamine (Q), leucine (L)
(viii) X₁₄ is selected from Glycine (G), threonine (T), serine (S).

In a preferred embodiment, X₁ is threonine (T), X₃ is proline (P), and X₅ is histidine (H), X₇ is aspartic acid (D), X₈ is valine (V), X₉ is glutamine (Q), X₁₀ is glycine (G), X₁₁ is alanine (A), X₁₂valine (V), X₁₃ is glutamine (Q), X₁₄ is Glycine (G), P₁ is thus SEQ ID NO: 19

In other embodiments, the present invention relates to a peptide or peptide analogue (P₁) comprising or consisting of the sequence

(SEQ ID NO: 12)
KEQEMX₁X₂X₃VX₄X₅X₆EX₇X₈X₉X₁₀X₁₁X₁₂X₁₃X₁₄.

In a preferred embodiment, X₁ is threonine (T), X₃ is proline (P), and X₅ is histidine (H), X₇ is aspartic acid (D), X₈ is valine (V), X₉ is glutamine (Q), X₁₀ is glycine (G), X₁₁ is alanine (A), X₁₂valine (V), X₁₃ is glutamine (Q), X₁₄ is Glycine (G), P₁ is thus SEQ ID NO: 22

In all embodiments of the present invention, the amino acid sequence surrounding amino acids in positions X₂, X₄, and X₆ is a characterizing feature of the peptide or peptide analogue as described herein. In some embodiments, the sequence of the eight amino acid residues comprising X₂, X₄, and X₆ is X₁X₂X₃VX₄X₅X₆E (SEQ ID NO: 9). In other embodiments, the sequence of thirteen amino acids residues comprising X₂, X₄, and X₆ is KEQEMX₁X₂X₃VX₄X₅X₆E (SEQ ID NO: 10). In other embodiments, the sequence of the sixteen amino acid residues comprising X₂, X₄, and X₆ is X₁X₂X₃VX₄X₅X₆EX₇X₈X₉X₁₀X₁₁X₁₂X₁₃X₁₄(SEQ ID NO: 11). In other embodiments, the sequence of the 21 amino acid residues comprising X₂, X₄, and X₆ is

(SEQ ID NO: 12)
KEQEMX₁X₂X₃VX₄X₅X₆EX₇X₈X₉X₁₀X₁₁X₁₂X₁₃X₁₄.

In a preferred embodiment, P₁ is the sequence of the eight amino acid residues comprising X₂, X₄, and X₆, and is X₁X₂X₃VX₄X₅X₆E (SEQ ID NO: 9), wherein X₁ is threonine (T), X₃ is proline (P), and X₅ is histidine (H), and P₁ is thus SEQ ID NO: 13.

In other embodiments, P₁ is the sequence of the eight amino acid residues comprising X₂, X₄, and X₆, and is X₁X₂X₃VX₄X₅X₆E (SEQ ID NO: 9), wherein X₁ is isoleucine (I), X₃ is proline (P), and X₅ is histidine (H), and P₁ is thus SEQ ID NO: 14.

In other embodiments, P₁ is the sequence of the eight amino acid residues comprising X₂, X₄, and X₆, and is X₁X₂X₃VX₄X₅X₆E (SEQ ID NO: 9), wherein X₁ is isoleucine (I), X₃ is serine (S), and X₅ is glutamine (Q), and P₁ is thus SEQ ID NO: 15.

In some embodiments, P₁ is the sequence of thirteen amino acids residues comprising X₂, X₄, and X₆, and is KEQEMX₁X₂X₃VX₄X₅X₆E (SEQ ID NO: 10), wherein X₁ is threonine (T), X₃ is proline (P), and X₅ is histidine (H), and P₁ is thus SEQ ID NO: 16.

In other embodiments, P₁ is the sequence of thirteen amino acids residues comprising X₂, X₄, and X₆, and is KEQEMX₁X₂X₃VX₄X₅X₆E (SEQ ID NO: 10), wherein X₁ is isoleucine (I), X₃ is proline (P), and X₅ is histidine (H), and P₁ is thus SEQ ID NO: 17.

In other embodiments, P₁ is the sequence of thirteen amino acids residues comprising X₂, X₄, and X₆, and is KEQEMX₁X₂X₃VX₄X₅X₆E (SEQ ID NO: 10), wherein X₁ is isoleucine (I), X₃ is serine (S), and X₅ is glutamine (Q), and P₁ is thus SEQ ID NO: 18.

In some embodiments, P₁ is the sequence of the sixteen amino acid residues comprising X₂, X₄, and X₆, and is X₁X₂X₃VX₄X₅X₆EX₇X₈X₉X₁₀X₁₁X₁₂X₁₃X₁₄(SEQ ID NO: 11), wherein X₁ is threonine (T), X₃ is proline (P), and X₅ is histidine (H), X₇ is aspartic acid (D), X₈ is valine (V), X₉ is glutamine (Q), X₁₀ is glycine (G), X₁₁ is alanine (A), X₁₂ valine (V), X₁₃ is glutamine (Q), X₁₄ is Glycine (G), and P₁ is thus SEQ ID NO: 19

In other embodiments, P₁ is the sequence of the sixteen amino acid residues comprising X₂, X₄, and X₆, and is X₁X₂X₃VX₄X₅X₆EX₇X₈X₉X₁₀X₁₁X₁₂X₁₃X₁₄(SEQ ID NO: 11), wherein X₁ is isoleucine (I), X₃ is proline (P), and X₅ is histidine (H), X₇ is serine (5), X₈ is arginine (R), X₉ is proline (P), X₁₀ is asparagine (N), X₁₁ is valine (V), X₁₂ proline (P), X₁₃ is glutamine (Q), X₁₄ is throenine (T), and P₁ is thus SEQ ID NO: 20.

In other embodiments, P₁ is the sequence of the sixteen amino acid residues comprising X₂, X₄, and X₆, and is X₁X₂X₃VX₄X₅X₆EX₇X₈X₉X₁₀X₁₁X₁₂X₁₃X₁₄(SEQ ID NO: 11), wherein X₁ is isoleucine (I), X₃ is serine (S), and X₅ is glutamine (Q), X₇ is asparagine (N), X₈ is alanine (A), X₉ is proline (P), X₁₀ is lysine (K), X₁₁ is isoleucine (I), X₁₂ threonine (T), X₁₃ is leucine (L), X₁₄ is serine (S), and P₁ is thus SEQ ID NO: 21.

In some embodiments, P₁ is the sequence of the 21 amino acid residues comprising X₂, X₄, and X₆, and is KEQEMX₁X₂X₃VX₄X₅X₆EX₇X₈X₉X₁₀X₁₁X₁₂X₁₃X₁₄ (SEQ ID NO: 12), wherein X₁ is threonine (T), X₃ is proline (P), and X₅ is histidine (H), X₇ is aspartic acid (D), X₈ is valine (V), X₉ is glutamine (Q), X₁₀ is glycine (G), X₁₁ is alanine (A), X₁₂ valine (V), X₁₃ is glutamine (Q), X₁₄ is Glycine (G), and P₁ is thus SEQ ID NO: 22.

In other embodiments, P₁ is the sequence of the 21 amino acid residues comprising X₂, X₄, and X₆, and is KEQEMX₁X₂X₃VX₄X₅X₆EX₇X₈X₉X₁₀X₁₁X₁₂X₁₃X₁₄ (SEQ ID NO: 12), wherein X₁ is isoleucine (I), X₃ is proline (P), and X₅ is histidine (H), X₇ is serine (S), X₈ is arginine (R), X₉ is proline (P), X₁₀ is asparagine (N), X₁₁ is valine (V), X₁₂ proline (P), X₁₃ is glutamine (Q), X₁₄ is throenine (T), and P₁ is thus SEQ ID NO: 23.

In other embodiments, P₁ is the sequence of the 21 amino acid residues comprising X₂, X₄, and X₆, and is KEQEMX₁X₂X₃VX₄X₅X₆EX₇X₈X₉X₁₀X₁₁X₁₂X₁₃X₁₄ (SEQ ID NO: 12), wherein X₁ is isoleucine (I), X₃ is serine (S), and X₅ is glutamine (Q), X₇ is asparagine (N), X₈ is alanine (A), X₉ is proline (P), X₁₀ is lysine (K), X₁₁ is isoleucine (I), X₁₂ threonine (T), X₁₃ is leucine (L), X₁₄ is serine (S), and P₁ is thus SEQ ID NO: 24.

According to all embodiments of the present invention, the identity of the amino acids in positions X₂, X₄, and X₆ are a characterizing feature of the peptide or peptide analogue as described herein. In one aspect of the present invention, the peptide or peptide analogue P₁ is useful for decreasing synaptic plasticity. This may be achieved with the use of the peptide or peptide analogue as described herein above wherein at least one serine (S) in positions X₂, X₄, X₆ has been changed to another residue. Accordingly, in some embodiments the sequence of the peptide or peptide analogue P₁ comprises or consists of at least one of X₂, X₄, X₆ wherein
(i) X₂ is selected from the group consisting of alanine (A), glycine (G), leucine (L), isoleucine (I), valine (V), proline (P), methionine (M),
(ii) X₄ is selected from the group consisting of alanine (A), glycine (G), leucine (L), isoleucine (I), valine (V), proline (P), methionine (M), or
(iii) X₆ is selected from the group consisting of alanine (A), glycine (G), leucine (L), isoleucine (I), valine (V), proline (P), methionine (M).

In such embodiments, X₄, and X₆ are preferably alanine (A), and P₁ is thus selected from the group consisting of SEQ ID NO: 50-74

Alternatively in such embodiments, X₂, X₄ and X₆ are alanine (A), and P₁ is thus selected from the group consisting of SEQ ID NO: 50-74

In one embodiment, P₁ is able to inhibit kinase activity and/or increase phosphatase activity.

In a preferred embodiment, P₁ is the sequence of the eight amino acid residues comprising X₂, X₄, and X₆, and is X₁X₂X₃VX₄X₅X₆E (SEQ ID NO: 9), wherein X₁ is threonine (T), X₂ is alanine (A), X₃ is proline (P), X₄ is alanine, X₅ is histidine (H), X₆ is alanine, and P₁ is thus SEQ ID NO: 62.

In such embodiments, the peptide or peptide analogue P₁ is referred to herein as an “inhibiting” peptide, and may be any one of peptides that inhibit synaptic plasticity, inhibit phosphorylation and/or inhibit changes in neural cell morphology such as reducing dendritic and/or axonal branching as described herein above in section I “Synaptic plasticity”.

In one embodiment, changes in neural morphology consist in increasing dendritic and/or axonal branching. In one embodiment, changes in neural morphology consist in decreasing or preventing increased of dendritic and/or axonal branching.

In another aspect of the present invention, the peptide or peptide analogue P₁ as defined herein, is useful for increasing synaptic plasticity. This may be achieved with the use of the peptide or peptide analogue as described herein below wherein at least one serine (S) in positions X₂, X₄, X₆ has been changed to another residue. Accordingly, in some embodiments the peptide or peptide analogue P₁ comprises or consists of at least one of X₂, X₄, X₆ wherein
(i) X₂ is selected from the group consisting of aspartic acid (D), glutamic acid (E), phosphoserine (J),
(ii) X₄ is selected from the group consisting of aspartic acid (D), glutamic acid (E), phosphoserine (J), or
(iii) X₆ is selected from the group consisting of aspartic acid (D), glutamic acid (E), phosphoserine (J).

In such embodiments, X₄ and X₆ are preferably aspartic acid (D), and P₁ is thus selected from the group consisting of SEQ ID NO: 25-49

Alternatively in such embodiments, X₂, X₄ and X₆ are aspartic acid (D), and P₁ is thus selected from the group consisting of SEQ ID NO: 25-49.

In other such embodiments, X₄ and X₆ are phosphoserine (J), and P₁ is thus selected from the group consisting of SEQ ID NO: 25-49.

In other such embodiments, X₂, X₄ and X₆ are phosphoserine (J), and P₁ is thus selected from the group consisting of SEQ ID NO: 25-49.

In yet other such embodiments, X₂, X₄, and X₆ are a combination of serine (S), aspartic acid (D) and phosphoserine (J) and P₁ is thus selected from the group consisting of SEQ ID NO: 25-49.

In some embodiments, P₁ is able to inhibit phosphatase activity and/or increase kinase activity.

In a preferred embodiment, kinase activity is increased and/or phosphatase activity is inhibited without the contribution of exogenous BDNF.

In a preferred embodiment, P₁ is the sequence of the eight amino acid residues comprising X₂, X₄, and X₆, and is X₁X₂X₃VX₄X₅X₆E (SEQ ID NO: 9), wherein X₁ is threonine (T), X₂ is aspartic acid (D), X₃ is proline (P), X₄ is aspartic acid (D), X₅ is histidine (H), X₆ is aspartic acid (D), and P₁ is thus SEQ ID NO: 37.

In such embodiments, the peptide or peptide analogue P₁ is referred to herein as an activating peptide, and may be any one of peptides that increase synaptic plasticity, increase phosphorylation and/or increase changes in neural cell morphology such as increasing dendritic and/or axonal branching as described herein above in section I “Synaptic plasticity”.

In some embodiments, the peptide or peptide analogue P₁ is a peptide sequence variant of amino acids 1100 to 1173 of any one of SEQ ID NOs: 1 to 8.

The peptide or peptide analogue P₁ as defined herein above may comprise between 8 and 76 amino acid residues, such as at least 9 amino acid residues, such as at least 10 amino acid residues, such as at least 11 amino acid residues, such as at least 12 amino acid residues, such as at least 13 amino acid residues, such as at least 14 amino acid residues, such as at least 15 amino acid residues, such as at least 16 amino acid residues, such as at least 17 amino acid residues, such as at least 18 amino acid residues, such as at least 19 amino acid residues, such as at least 20 amino acid residues, such as at least 21 amino acid residues, such as at least 22 amino acid residues, such as at least 23 amino acid residues, such as at least 24 amino acid residues, such as at least 25 amino acid residues, such as at least 26 amino acid residues, such as at least 27 amino acid residues, such as at least 28 amino acid residues, such as at least 29 amino acid residues, such as at least 30 amino acid residues, such as at least 31 amino acid residues, such as at least 32 amino acid residues, such as at least 33 amino acid residues, such as at least 34 amino acid residues, such as at least 35 amino acid residues, such as at least 36 amino acid residues, such as at least 40 amino acid residues, such as at least 45 amino acid residues, such as at least 50 amino acid residues, such as at least 55 amino acid residues, such as at least 60 amino acid residues, such as at least 65 amino acid residues, such as at least 70 amino acid residues, such as at least 75 amino acid residues, such as 76 amino acid residues.

In some embodiments, the peptide or peptide analogue consists of 16 amino acid residues comprising the 8 amino acid sequence X₁X₂X₃VX₄X₅X₆E (SEQ ID NO: 9) or the 13 amino acid sequence KEQEMX₁X₂X₃VX₄X₅X₆E (SEQ ID NO: 10) or the 16 amino acid sequence X₁X₂X₃VX₄X₅X₆EX₇X₈X₉X₁₀X₁₁X₁₂ X₁₃X₁₄ (SEQ ID NO: 11). In other embodiments, the peptide or peptide analogue consists of 17 amino acid residues comprising the 8 amino acid sequence X₁X₂X₃VX₄X₅X₆E (SEQ ID NO: 9) or the 13 amino acid sequence KEQEMX₁X₂X₃VX₄X₅X₆E (SEQ ID NO: 10) or the 16 amino acid sequence X₁X₂X₃VX₄X₅X₆EX₇X₈X₉X₁₀X₁₁X₁₂ X₁₃X₁₄ (SEQ ID NO: 11). In other embodiments, the peptide or peptide analogue consists of 18 amino acid residues comprising the 8 amino acid sequence X₁X₂X₃VX₄X₅X₆E (SEQ ID NO: 9) or the 13 amino acid sequence KEQEMX₁X₂X₃VX₄X₅X₆E (SEQ ID NO: 10) or the 16 amino acid sequence X₁X₂X₃VX₄X₅X₆EX₇X₈X₉X₁₀X₁₁X₁₂ X₁₃X₁₄ (SEQ ID NO: 11). In other embodiments, the peptide or peptide analogue consists of 19 amino acid residues comprising the 8 amino acid sequence X₁X₂X₃VX₄X₅X₆E (SEQ ID NO: 9) or the 13 amino acid sequence KEQEMX₁X₂X₃VX₄X₅X₆E (SEQ ID NO: 10) or the 16 amino acid sequence X₁X₂X₃VX₄X₅X₆EX₇X₈X₉X₁₀X₁₁X₁₂ X₁₃X₁₄ (SEQ ID NO: 11). In other embodiments, the peptide or peptide analogue consists of 20 amino acid residues comprising the 8 amino acid sequence X₁X₂X₃VX₄X₅X₆E (SEQ ID NO: 9) or the 13 amino acid sequence KEQEMX₁X₂X₃VX₄X₅X₆E (SEQ ID NO: 10) or the 16 amino acid sequence X₁X₂X₃VX₄X₅X₆EX₇X₈X₉X₁₀X₁₁X₁₂ X₁₃X₁₄ (SEQ ID NO: 11). In other embodiments, the peptide or peptide analogue consists of 21 amino acid residues comprising the 8 amino acid sequence X₁X₂X₃VX₄X₅X₆E (SEQ ID NO: 9) or the 13 amino acid sequence KEQEMX₁X₂X₃VX₄X₅X₆E (SEQ ID NO: 10) or the 16 amino acid sequence X₁X₂X₃VX₄X₅X₆EX₇X₈X₉X₁₀X₁₁X₁₂ X₁₃X₁₄ (SEQ ID NO: 11) or the 21 amino acid sequence KEQEMX₁X₂X₃VX₄X₅X₆EX₇X₈X₉X₁₀X₁₁X₁₂X₁₃X₁₄ (SEQ ID NO: 12). In other embodiments, the peptide or peptide analogue consists of 22 amino acid residues comprising the 8 amino acid sequence X₁X₂X₃VX₄X₅X₆E (SEQ ID NO: 9) or the 13 amino acid sequence KEQEMX₁X₂X₃VX₄X₅X₆E (SEQ ID NO: 10) or the 16 amino acid sequence X₁X₂X₃VX₄X₅X₆EX₇X₈X₉X₁₀X₁₁X₁₂ X₁₃X₁₄ (SEQ ID NO: 11) or the 21 amino acid sequence KEQEMX₁X₂X₃VX₄X₅X₆EX₇X₈X₉X₁₀X₁₁X₁₂X₁₃X₁₄ (SEQ ID NO: 12). In other embodiments, the peptide or peptide analogue consists of 23 amino acid residues comprising the 8 amino acid sequence X₁X₂X₃VX₄X₅X₆E (SEQ ID NO: 9) or the 13 amino acid sequence KEQEMX₁X₂X₃VX₄X₅X₆E (SEQ ID NO: 10) or the 16 amino acid sequence X₁X₂X₃VX₄X₅X₆EX₇X₈X₉X₁₀X₁₁X₁₂ X₁₃X₁₄ (SEQ ID NO: 11) or the 21 amino acid sequence KEQEMX₁X₂X₃VX₄X₅X₆EX₇X₈X₉X₁₀X₁₁X₁₂X₁₃X₁₄ (SEQ ID NO: 12). In other embodiments, the peptide or peptide analogue consists of 24 amino acid residues comprising the 8 amino acid sequence X₁X₂X₃VX₄X₅X₆E (SEQ ID NO: 9) or the 13 amino acid sequence KEQEMX₁X₂X₃VX₄X₅X₆E (SEQ ID NO: 10) or the 16 amino acid sequence X₁X₂X₃VX₄X₅X₆EX₇X₈X₉X₁₀X₁₁X₁₂ X₁₃X₁₄ (SEQ ID NO: 11) or the 21 amino acid sequence KEQEMX₁X₂X₃VX₄X₅X₆EX₇X₈X₉X₁₀X₁₁X₁₂X₁₃X₁₄ (SEQ ID NO: 12). In other embodiments, the peptide or peptide analogue consists of 25 amino acid residues comprising the 8 amino acid sequence X₁X₂X₃VX₄X₅X₆E (SEQ ID NO: 9) or the 13 amino acid sequence KEQEMX₁X₂X₃VX₄X₅X₆E (SEQ ID NO: 10) or the 16 amino acid sequence X₁X₂X₃VX₄X₅X₆EX₇X₈X₉X₁₀X₁₁X₁₂ X₁₃X₁₄ (SEQ ID NO: 11) or the 21 amino acid sequence KEQEMX₁X₂X₃VX₄X₅X₆EX₇X₈X₉X₁₀X₁₁X₁₂X₁₃X₁₄ (SEQ ID NO: 12). In other embodiments, the peptide or peptide analogue consists of 26 amino acid residues comprising the 8 amino acid sequence X₁X₂X₃VX₄X₅X₆E (SEQ ID NO: 9) or the 13 amino acid sequence KEQEMX₁X₂X₃VX₄X₅X₆E (SEQ ID NO: 10) or the 16 amino acid sequence X₁X₂X₃VX₄X₅X₆EX₇X₈X₉X₁₀X₁₁X₁₂ X₁₃X₁₄ (SEQ ID NO: 11) or the 21 amino acid sequence KEQEMX₁X₂X₃VX₄X₅X₆EX₇X₈X₉X₁₀X₁₁X₁₂X₁₃X₁₄ (SEQ ID NO: 12). In other embodiments, the peptide or peptide analogue consists of 27 amino acid residues comprising the 8 amino acid sequence X₁X₂X₃VX₄X₅X₆E (SEQ ID NO: 9) or the 13 amino acid sequence KEQEMX₁X₂X₃VX₄X₅X₆E (SEQ ID NO: 10) or the 16 amino acid sequence X₁X₂X₃VX₄X₅X₆EX₇X₈X₉X₁₀X₁₁X₁₂ X₁₃X₁₄ (^{SEQ} ID NO: 11) or the 21 amino acid sequence KEQEMX₁X₂X₃VX₄X₅X₆EX₇X₈X₉X₁₀X₁₁X₁₂X₁₃X₁₄ (SEQ ID NO: 12). In other embodiments, the peptide or peptide analogue consists of 28 amino acid residues comprising the 8 amino acid sequence X₁X₂X₃VX₄X₅X₆E (SEQ ID NO: 9) or the 13 amino acid sequence KEQEMX₁X₂X₃VX₄X₅X₆E (SEQ ID NO: 10) or the 16 amino acid sequence X₁X₂X₃VX₄X₅X₆EX₇X₈X₉X₁₀X₁₁X₁₂ X₁₃X₁₄ (SEQ ID NO: 11) or the 21 amino acid sequence KEQEMX₁X₂X₃VX₄X₅X₆EX₇X₈X₉X₁₀X₁₁X₁₂X₁₃X₁₄ (SEQ ID NO: 12). In other embodiments, the peptide or peptide analogue consists of 29 amino acid residues comprising the 8 amino acid sequence X₁X₂X₃VX₄X₅X₆E (SEQ ID NO: 9) or the 13 amino acid sequence KEQEMX₁X₂X₃VX₄X₅X₆E (SEQ ID NO: 10) or the 16 amino acid sequence X₁X₂X₃VX₄X₅X₆EX₇X₈X₉X₁₀X₁₁X₁₂ X₁₃X₁₄ (SEQ ID NO: 11) or the 21 amino acid sequence KEQEMX₁X₂X₃VX₄X₅X₆EX₇X₈X₉X₁₀X₁₁X₁₂X₁₃X₁₄ (SEQ ID NO: 12). In other embodiments, the peptide or peptide analogue consists of 30 amino acid residues comprising the 8 amino acid sequence X₁X₂X₃VX₄X₅X₆E (SEQ ID NO: 9) or the 13 amino acid sequence KEQEMX₁X₂X₃VX₄X₅X₆E (SEQ ID NO: 10) or the 16 amino acid sequence X₁X₂X₃VX₄X₅X₆EX₇X₈X₉X₁₀X₁₁X₁₂ X₁₃X₁₄ (SEQ ID NO: 11) or the 21 amino acid sequence KEQEMX₁X₂X₃VX₄X₅X₆EX₇X₈X₉X₁₀X₁₁X₁₂X₁₃X₁₄ (SEQ ID NO: 12). In other embodiments, the peptide or peptide analogue consists of 31 amino acid residues comprising the 8 amino acid sequence X₁X₂X₃VX₄X₅X₆E (SEQ ID NO: 9) or the 13 amino acid sequence KEQEMX₁X₂X₃VX₄X₅X₆E (SEQ ID NO: 10) or the 16 amino acid sequence X₁X₂X₃VX₄X₅X₆EX₇X₈X₉X₁₀X₁₁X₁₂ X₁₃X₁₄ (SEQ ID NO: 11) or the 21 amino acid sequence KEQEMX₁X₂X₃VX₄X₅X₆EX₇X₈X₉X₁₀X₁₁X₁₂X₁₃X₁₄ (SEQ ID NO: 12). In other embodiments, the peptide or peptide analogue consists of 32 amino acid residues comprising the 8 amino acid sequence X₁X₂X₃VX₄X₅X₆E (SEQ ID NO: 9) or the 13 amino acid sequence KEQEMX₁X₂X₃VX₄X₅X₆E (SEQ ID NO: 10) or the 16 amino acid sequence X₁X₂X₃VX₄X₅X₆EX₇X₈X₉X₁₀X₁₁X₁₂ X₁₃X₁₄ (SEQ ID NO: 11) or the 21 amino acid sequence KEQEMX₁X₂X₃VX₄X₅X₆EX₇X₈X₉X₁₀X₁₁X₁₂X₁₃X₁₄ (SEQ ID NO: 12). In other embodiments, the peptide or peptide analogue consists of 33 amino acid residues comprising the 8 amino acid sequence X₁X₂X₃VX₄X₅X₆E (SEQ ID NO: 9) or the 13 amino acid sequence KEQEMX₁X₂X₃VX₄X₅X₆E (SEQ ID NO: 10) or the 16 amino acid sequence X₁X₂X₃VX₄X₅X₆EX₇X₈X₉X₁₀X₁₁X₁₂ X₁₃X₁₄ (SEQ ID NO: 11) or the 21 amino acid sequence KEQEMX₁X₂X₃VX₄X₅X₆EX₇X₈X₉X₁₀X₁₁X₁₂X₁₃X₁₄ (SEQ ID NO: 12). In other embodiments, the peptide or peptide analogue consists of 34 amino acid residues comprising the 8 amino acid sequence X₁X₂X₃VX₄X₅X₆E (SEQ ID NO: 9) or the 13 amino acid sequence KEQEMX₁X₂X₃VX₄X₅X₆E (SEQ ID NO: 10) or the 16 amino acid sequence X₁X₂X₃VX₄X₅X₆EX₇X₈X₉X₁₀X₁₁X₁₂ X₁₃X₁₄ (SEQ ID NO: 11) or the 21 amino acid sequence KEQEMX₁X₂X₃VX₄X₅X₆EX₇X₈X₉X₁₀X₁₁X₁₂X₁₃X₁₄ (SEQ ID NO: 12). In other embodiments, the peptide or peptide analogue consists of 35 amino acid residues comprising the 8 amino acid sequence X₁X₂X₃VX₄X₅X₆E (SEQ ID NO: 9) or the 13 amino acid sequence KEQEMX₁X₂X₃VX₄X₅X₆E (SEQ ID NO: 10) or the 16 amino acid sequence X₁X₂X₃VX₄X₅X₆EX₇X₈X₉X₁₀X₁₁X₁₂ X₁₃X₁₄ (SEQ ID NO: 11) or the 21 amino acid sequence KEQEMX₁X₂X₃VX₄X₅X₆EX₇X₈X₉X₁₀X₁₁X₁₂X₁₃X₁₄ (SEQ ID NO: 12). In other embodiments, the peptide or peptide analogue consists of 36 amino acid residues comprising the 8 amino acid sequence X₁X₂X₃VX₄X₅X₆E (SEQ ID NO: 9) or the 13 amino acid sequence KEQEMX₁X₂X₃VX₄X₅X₆E (SEQ ID NO: 10) or the 16 amino acid sequence X₁X₂X₃VX₄X₅X₆EX₇X₈X₉X₁₀X₁₁X₁₂ X₁₃X₁₄ (SEQ ID NO: 11) or the 21 amino acid sequence KEQEMX₁X₂X₃VX₄X₅X₆EX₇X₈X₉X₁₀X₁₁X₁₂X₁₃X₁₄ (SEQ ID NO: 12). In other embodiments, the peptide or peptide analogue consists of at most 76 amino acid residues comprising the 8 amino acid sequence X₁X₂X₃VX₄X₅X₆E (SEQ ID NO: 9) or the 13 amino acid sequence KEQEMX₁X₂X₃VX₄X₅X₆E (SEQ ID NO: 10) or the 16 amino acid sequence X₁X₂X₃VX₄X₅X₆EX₇X₈X₉X₁₀X₁₁X₁₂ X₁₃X₁₄ (SEQ ID NO: 11) or the 21 amino acid sequence KEQEMX₁X₂X₃VX₄X₅X₆EX₇X₈X₉X₁₀X₁₁X₁₂X₁₃X₁₄ (SEQ ID NO: 12).

In certain embodiments, it is advantageous to enhance the capability of the peptides of the present invention to pass the plasma membrane of a cell. This can be achieved by conjugating a functional moiety to the peptides.

Accordingly, in some embodiments of the present invention, the peptide or peptide analogue described herein may be further modified, for example may be conjugated to one or more moieties that add further features. Said conjugated moieties may facilitate penetration of the peptide or peptide analogue through a membrane. Said conjugated moieties may also allow easy detection of the peptide or peptide analogue. Examples of moieties that can be conjugated to the peptide or peptide analogue described herein are found in the section below “Conjugated moiety”. A non-limiting list of examples of conjugated moieties comprises a cell penetrating peptide (CPP), an albumin binding domain (ABM) and a detectable moiety.

Accordingly, in some embodiments the peptide or peptide analogue P₁ may be conjugated to at least one additional moiety, such as to two conjugated moieties, (Y₁) and (Y₂), wherein these are selected from the group consisting of a Cell Penetrating Peptide (CPP), an Albumin Binding Moiety (ABM), and a detectable moiety (Z).

In some embodiments, the peptide or peptide analogue P₁ has an additional moiety comprising a CPP, the CPP comprising a sequence of at least four residues selected from arginine (R) and lysine (Y). In other embodiments, the CPP comprises a retroinverso peptide or a Tat peptide having amino acid sequence GRKKRRQRRR or a Retroinverso-d-Tat peptide having amino acid sequence of rrrqrrkkrg.

In one embodiment, the CPP comprises 5 arginine residues. In another embodiment, the 5 arginine residues are conjugated to the C-terminus of the peptide or peptide analogue. The CPP may be conjugated to the peptide or peptide analogue by an amide bond. Accordingly, in some embodiments, the peptide or peptide analogue comprises or consists of the sequence Z₁Z₂Z₃Z₄X₁X₂X₃VX₄X₅X₆E or X₁X₂X₃VX₄X₅X₆EZ₁Z₂Z₃Z₄ (SEQ ID NO: 75), wherein X₁, X₂, X₃, X₄, X₅, and X₆ are as defined above and Z₁, Z₂, Z₃, and Z₄ are individually selected from R and K.

According to some embodiments, the peptide or peptide analogue P₁ may further comprise a detectable moiety allowing in vitro and/or in vivo detection. Said detectable moiety is in some embodiments selected from a group comprising fluorophores, chromophores and radioactive compounds as described in detail in the section “Conjugated moieties” below. In one embodiment, said detectable moiety is a fluorophore. In another embodiment, said detectable moiety is the fluorophore 5/6 carboxy-tetramethyl rhodamine (TMR).

In some embodiments of the present invention, the peptide or peptide analogue may be comprised in a pharmaceutically acceptable composition and is useful for use as a medicament for the treatment and/or prevention of diseases of the nervous system. The peptide or peptide analogue of the present invention may also be useful for the treatment and prevention of mental and behavioural disorders, neurodevelopmental congenital malformations and chromosomal abnormalities, cardiovascular disorders including vascular syndromes of brain in cerebrovascular diseases, metabolic and eating disorders and neoplastic disorders.

Conjugated Moiety

The peptide or peptide analogue as described in the present invention may be further conjugated to an additional moiety (Y). Said moiety may be conjugated to a terminal end of the peptide or peptide analogue.

In one embodiment, said conjugated moiety is a peptide, which may be linked via an amide bond to the peptide or peptide analogue via an amide bond. Said conjugated peptide may be a cell penetrating peptide (CPP) facilitating penetration of the composition through e.g. a cellular membrane. Said conjugated peptide may comprise at least 4 amino acid residues selected from a group comprising arginine and lysine. In another embodiment of the present invention, said conjugated peptide comprises 5 arginine residues. In a further embodiment, said conjugated peptide comprises at the most 13 amino acid residues.

In other embodiments, said conjugated moiety is an albumin binding moiety (ABM) herein defined as any suitable chemical group binding albumin. A non-limiting example of a suitable ABM is a fatty acid, but other chemical groups may be used. In some embodiments the ABM is conjugated to the linker in the dimeric peptide or peptide analogue.

In one embodiment, said conjugated moiety is a detectable moiety (Z), wherein said moiety may be detected using a technology based on fluorescence or on radioactivity or UV spectrometry. Said detectable moiety may be conjugated to an N-terminus of the peptide or peptide analogue. Said detectable moiety (Z) may also be conjugated to the C-terminus of a CPP.

The peptide or peptide analogue as described herein may be conjugated to a detectable moiety, for example to a fluorophore. Suitable fluorophores are known to the skilled person.

In some embodiments the fluorophore is TMR. Said fluorophore may be detected via conventional fluorescence microscopy. In other embodiments the detectable moiety is a dye that can be used in super-resolution microscopy. A non-limiting list of examples of detectable moieties comprises Alexa Fluor® 488, Alexa Fluor® 532, Alexa Fluor® 568, Alexa Fluor® 647, ATTO 488 and ATTO 532. Said fluorophores may be detected via super-resolution microscopy.

In one embodiment, the presence of said detectable moiety allows detection of the peptide or peptide analogue comprised in the composition under in-vitro and/or in-vivo conditions. Accordingly, in a further embodiment, said detectable moiety allows indirect detection of any compound binding to the peptide or peptide analogue in-vitro and or in-vivo. Said other compounds may be proteins that interact with the peptide or peptide analogue. In one embodiment, said other compounds may be synaptic proteins, i.e. proteins that in a natural context are to be found within the inter-synaptic space existing between two synapses, whether secreted by surrounding neurons or other cell-types.

The peptide or peptide analogue may also be conjugated to a polymer. Said polymer may be a resin, wherein said resin may be contacted with a mixture comprising proteins. In one embodiment of the present invention, said resin is in the form of beads. The peptides or peptide analogues conjugated to the resin may bind to a protein comprised in said mixture, thereby allowing their isolation. Said peptide or peptide analogue may be conjugated to the resin via a terminal amino acid residue. In one embodiment of the present invention, the peptide or peptide analogue is conjugated to said resin via a terminal cysteine residue. In another embodiment of the present invention, the peptide or peptide analogue is conjugated to the resin via an amino acid residue situated at the C-terminus of the CPP. Said residue may be a cysteine. Further, the peptide or peptide analogue may be conjugated to the resin covalently, e.g. via thiol-based chemistry.

### III. Polynucleotides, Vectors and Host Cells

The invention is as defined in the claims.

In some embodiments of the present invention, the peptide P₁ defined herein is encoded by a polynucleotide. It is to be understood that said polynucleotide encoding a polypeptide (also referred to as “coding sequence” in the following) is operably linked in sense orientation to one or more regulatory regions suitable for directing expression of the polypeptide. A coding sequence and a regulatory region are considered to be operably linked when the regulatory region and coding sequence are positioned so that the regulatory region is effective for regulating transcription or translation of the sequence.

“Regulatory region” refers to a nucleic acid having nucleotide sequences that influence transcription or translation initiation and rate, and stability and/or mobility of a transcription or translation product. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, introns, and combinations thereof. A regulatory region typically comprises at least a core (basal) promoter. A regulatory region also may include at least one control element, such as an enhancer sequence, an upstream element or an upstream activation region (UAR). A regulatory region is operably linked to a coding sequence by positioning the regulatory region and the coding sequence so that the regulatory region is effective for regulating transcription or translation of the sequence.

The choice of regulatory regions to be included depends upon several factors, including the type of host cell. It is a routine matter for one of skill in the art to modulate the expression of a coding sequence by appropriately selecting and positioning regulatory regions relative to the coding sequence. It will be understood that more than one regulatory region may be present, e.g., introns, enhancers, upstream activation regions, transcription terminators, and inducible elements.

It will be appreciated that because of the degeneracy of the genetic code, a number of nucleic acids can encode a particular polypeptide; i.e., for many amino acids, there is more than one nucleotide triplet that serves as the codon for the amino acid. Thus, codons in the coding sequence for a given polypeptide can be modified such that optimal expression in a particular host organisms obtained, using appropriate codon bias tables for that host cell (e.g., bacterial cell, mammalian cell, human cell). Nucleic acids may also be optimized to a GC-content preferable to a particular host, and/or to reduce the number of repeat sequences. As isolated nucleic acids, these modified sequences can exist as purified molecules and can be incorporated into a vector or a virus for use in constructing modules for recombinant nucleic acid constructs.

IV. Use of Peptides for the Treatment and/or Prevention of Diseases.

In one aspect of the present invention, the peptide or peptide analogue, the polynucleotide, the vector, the cell or composition as described herein is useful for use for the treatment and/or prevention of one or more diseases of the nervous system, such as systemic atrophies primarily affecting the central nervous system e.g. Huntington disease, and degenerative diseases of the central nervous system such as Alzheimer's disease, extrapyramidal and movement disorders of the nervous system such as Parkinson disease.

In another aspect of the present invention, the peptide or peptide analogue P₁, the polynucleotide, the vector, the cell or composition described herein is also useful for the treatment and/or prevention of one or more of mental and behavioural disorders, such as depression, anxiety, obsessive compulsive disorder (OCD), bipolar disorder (BD), Schizophrenia (SZ), and Rett syndrome.

In another aspect of the present invention, the peptide or peptide analogue, polynucleotide, vector, cell or composition of the present invention is also useful for the treatment and/or prevention of one or more of neurodevelopmental congenital malformations and chromosomal abnormalities, such as Rubinstein-Taybi Syndrome.

In another aspect of the present invention, the peptide or peptide analogue, polynucleotide, vector, cell or composition of the present invention is also useful for the treatment and/or prevention of one or more of cardiovascular disorders including vascular syndromes of brain in cerebrovascular diseases, such as stroke.

In another aspect of the present invention, the peptide or peptide analogue, polynucleotide, vector, cell or composition of the present invention is also useful for the treatment and/or prevention of one or more of metabolic and eating disorders and neoplastic disorders, such as diabetes, obesity, insulin resistance, and anorexia nervosa.

In such embodiments, said uses comprise the administration of the “activating” peptide or peptide analogue P₁ as described herein above in the section II “Peptide or peptide analogue”, a polynucleotide encoding said peptide, a vector comprising said polynucleotide, a host cell comprising said polynucleotide or vector as described herein above in section III “Polynucleotides, vectors and host cells”, or a composition comprising any one or more of the aforementioned as described in the section “Pharmaceutical compositions” to a subject in need thereof, i.e. subjects suffering from or suspected of suffering from said diseases.

In another aspect of the present invention, the peptide or peptide analogue, polynucleotide, vector, cell or composition of the present invention is also useful for the treatment and/or prevention of a malignant neoplastic disorder such as a genitourinary anomaly or gonadoblastoma or a neoplasm of the kidney.

In another aspect of the present invention, the peptide or peptide analogue, polynucleotide, vector, cell or composition of the present invention is also useful for the treatment and/or prevention of one or more of episodic and paroxysmal disorders of the nervous system diseases, such as those selected from a list consisting of epilepsy, status epilepticus and migraine.

In another aspect of the present invention, the peptide or peptide analogue, polynucleotide, vector, cell or composition of the present invention is also useful for the treatment and/or prevention of one or more of other nervous system disorders such as neuropathic pain and pain hypersensitivity induced by neuropathic pain.

In such embodiments, said uses comprise the administration of the “inhibiting” peptide or peptide analogue P₁ as described herein above in the section II “Peptide or peptide analogue”, a polynucleotide encoding said peptide, a vector comprising said polynucleotide, a host cell comprising said polynucleotide or vector as described herein above in section III “Polynucleotides, vectors and host cells”, or a composition comprising any one or more of the aforementioned as described in the section “Pharmaceutical compositions” to a subject in need thereof, i.e. subjects suffering from or suspected of suffering from said diseases.

In another aspect, said method of treatment of the diseases described herein above comprises the administration of a compound capable of modulating phosphorylation of the C-terminal domain of one or more Vps10 domain-containing receptors as described herein below in the section “C-terminal domain of SorCS2, SorCS1 or SorCS3”, to a subject in need thereof. Said compound may be the peptide or peptide analogue P₁ as described herein above in section II “Peptide or peptide analogue”, a polynucleotide encoding said peptide, a vector comprising said polynucleotide, a host cell comprising said polynucleotide or vector as described herein above in section III “Polynucleotides, vectors and host cells”, or a composition comprising any one or more of the aforementioned as described in the section “Pharmaceutical compositions”, but it may also be any other compound, in particular a compound identified according to the description herein below in the section “Method for identifying a compound capable of modulating phosphorylation of the C-terminal cytoplasmic domain of a Vps10p-domain receptor”.

In yet another aspect, the present invention relates to a method of increasing the number of synapses in a subject, said method comprising administering the peptide or peptide analogue P₁ as described herein above in section II “Peptide or peptide analogue”, a polynucleotide encoding said peptide, a vector comprising said polynucleotide, a host cell comprising said polynucleotide or vector as described herein above in section III “Polynucleotides, vectors and host cells”, or a composition comprising any one or more of the aforementioned as described in the section “Pharmaceutical compositions” or a compound capable of modulating phosphorylation of the C-terminal domain of SorCS2, SorCS1 or SorCS3 to a subject.

In yet another aspect, the present invention relates to a method of inducing phosphorylation of the C-terminal domain of SorCS2, SorCS1 or SorCS3 in primary hippocampal neuronal cells, said method comprising administering the peptide or peptide analogue P₁ as described herein above in section II “Peptide or peptide analogue”, a polynucleotide encoding said peptide, a vector comprising said polynucleotide, a host cell comprising said polynucleotide or vector as described herein above in section III “Polynucleotides, vectors and host cells”, or a composition comprising any one or more of the aforementioned as described in the section “Pharmaceutical compositions” or a compound capable of modulating phosphorylation of the C-terminal domain of SorCS2, SorCS1 or SorCS3.

In some embodiments, the neurological disorder or the mental and behavioral disorder is associated with impaired synaptic plasticity. In particular, the disorder may be associated with a deficiency in long-term potentiation (LTP), with a deficiency in long-term depression (LTD) or with a deficiency in both LTP and LTD. In some embodiments, the neurological disorder or mental and behavioral disorder is associated with deregulated BDNF-signaling.

Accordingly, the peptide or peptide analogue described herein may be a competitive inhibitor. In some embodiments, said peptide or peptide analogue is a competitive inhibitor of BDNF, thereby reducing synaptic plasticity and decreasing the amount of morphological changes in neurons, such as dendritic and axonal branching, and thus an “inhibitor” peptide as described herein above. In other embodiments, said peptide or peptide analogue is an activator of synaptic plasticity and increases the amount of morphological changes in neurons, such as dendritic and axonal branching, even without the contribution of exogenous BDNF, thereby substituting for the need of functional BDNF and/or BDNF signalling, and is thus an “activating” peptide as described herein above.

### V. Pharmaceutical Compositions

Whilst it is possible for the compounds or salts of the present invention to be administered as the raw peptide or peptide analogue, it is preferred to present them in the form of a pharmaceutical formulation. Accordingly, the present invention further provides a pharmaceutical formulation, which comprises a compound of the present invention or a pharmaceutically acceptable salt or ester thereof, as herein defined, and a pharmaceutically acceptable carrier therefor. The pharmaceutical formulations may be prepared by conventional techniques, e.g. as described in Remington: The Science and Practice of Pharmacy 2005, Lippincott, Williams & Wilkins.

The pharmaceutically acceptable carriers can be either solid or liquid. Solid form preparations include powders, tablets, pills, capsules, cachets, suppositories, and dispersible granules. A solid carrier can be one or more excipients which may also act as diluents, flavoring agents, solubilizers, lubricants, suspending agents, binders, preservatives, wetting agents, tablet disintegrating agents, or an encapsulating material.

Also included are solid form preparations, which are intended to be converted shortly before use to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The compounds of the present invention may be formulated for parenteral administration and may be presented in unit dose form in ampoules, pre-filled syringes, small volume infusion or in multi-dose containers, optionally with an added preservative. The compositions may take such forms as suspensions, solutions, or emulsions in oily or aqueous vehicles, for example solutions in aqueous polyethylene glycol. Examples of oily or non-aqueous carriers, diluents, solvents or vehicles include propylene glycol, polyethylene glycol, vegetable oils (e.g., olive oil), and injectable organic esters (e.g., ethyl oleate), and may contain agents such as preserving, wetting, emulsifying or suspending, stabilizing and/or dispersing agents. Alternatively, the active ingredient may be in powder form, obtained by aseptic isolation of sterile solid or by lyophilisation from solution for constitution before use with a suitable vehicle, e.g., sterile, pyrogen-free water.

Pharmaceutically acceptable salts of the instant compounds, where they can be prepared, are also intended to be covered by this invention. These salts will be ones which are acceptable in their application to a pharmaceutical use. By that it is meant that the salt will retain the biological activity of the parent compound and the salt will not have untoward or deleterious effects in its application and use in treating diseases.

Pharmaceutically acceptable salts are prepared in a standard manner. If the parent compound is a base it is treated with an excess of an organic or inorganic acid in a suitable solvent. If the parent compound is an acid, it is treated with an inorganic or organic base in a suitable solvent.

The compounds of the invention may be administered in the form of an alkali metal or earth alkali metal salt thereof, concurrently, simultaneously, or together with a pharmaceutically acceptable carrier or diluent, especially and preferably in the form of a pharmaceutical composition thereof, whether by oral, rectal, or parenteral (including subcutaneous) route, in an effective amount.

VI. C-terminal domain of SorCS2, SorCS1 or SorCS3.

The present invention relates to the finding that phosphorylation of the C-terminal domain of the Vps10 domain-containing receptor SorCS2, SorCS1 and/or SorCS3 is important for synaptic plasticity. SorCS2, SorCS1 and SorCS3 comprise an N-terminal Vps10p-domain with high homology to Vps1Op and a cytoplasmic C-terminal domain.

There are four natural forms of SorCS2, which are set out in SEQ ID NO: 1, SEQ ID NO: 3, SEQ ID NO: 5 and SEQ ID NO: 7. Throughout the present invention, the term “SorCS2” may refer to any of these forms as defined below, and the term “C-terminal domain of SorCS2” may refer to the C-terminal domain of any of these forms as defined below.

The first form (tail A) is set out in SEQ ID NO: 1. It is 1159 residues long, and the C-terminal domain in this form shall be defined as the region spanning from amino acid residues 1099, 1100 or 1101 to 1159 of SEQ ID NO: 1.

The second form (tail B) is set out in SEQ ID NO: 3. It is 1173 residues long, and includes an acid domain spanning from residues 1139 to 1152. The C-terminal domain in this form shall be defined as the region spanning from amino acid residues 1099, 1100 or 1101 to 1173 of SEQ ID NO: 3.

The third form (tail C) is set out in SEQ ID NO: 5. It is 1174 residues long, and includes an additional serine at position 1104, as well as the acid domain, which spans from residues 1140 to 1153 of SEQ ID NO: 5. The C-terminal domain in this form shall be defined as the region spanning from amino acid residues 1099, 1100 or 1101 to 1174 of SEQ ID NO: 5.

The fourth form (tail D) is set out in SEQ ID NO: 7. It is 1160 residues long, and includes the additional serine at position 1104; it is however devoid of the acid domain.

The C-terminal domain in this form shall be defined as the region spanning from amino acid residues 1099, 1100 or 1101 to 1160 of SEQ ID NO: 7.

Accordingly, in some embodiments, the C-terminal domain of SorCS2 corresponds to the region spanning from amino acid residues 1099 to 1159 of SEQ ID NO: 1. In some embodiments, the C-terminal domain of SorCS2 corresponds to the region spanning from amino acid residues 1100 to 1159 of SEQ ID NO: 1. In some embodiments, the C-terminal domain of SorCS2 corresponds to the region spanning from amino acid residues 1101 to 1159 of SEQ ID NO: 1.

In some embodiments, the C-terminal domain of SorCS2 corresponds to the region spanning from amino acid residues 1099 to 1173 of SEQ ID NO: 3. In some embodiments, the C-terminal domain of SorCS2 corresponds to the region spanning from amino acid residues 1100 to 1173 of SEQ ID NO: 3. In some embodiments, the C-terminal domain of SorCS2 corresponds to the region spanning from amino acid residues 1101 to 1173 of SEQ ID NO: 3.

In some embodiments, the C-terminal domain of SorCS2 corresponds to the region spanning from amino acid residues 1099 to 1174 of SEQ ID NO: 5. In some embodiments, the C-terminal domain of SorCS2 corresponds to the region spanning from amino acid residues 1100 to 1174 of SEQ ID NO: 5. In some embodiments, the C-terminal domain of SorCS2 corresponds to the region spanning from amino acid residues 1101 to 1174 of SEQ ID NO: 5.

In some embodiments, the C-terminal domain of SorCS2 corresponds to the region spanning from amino acid residues 1099 to 1160 of SEQ ID NO: 7. In some embodiments, the C-terminal domain of SorCS2 corresponds to the region spanning from amino acid residues 1100 to 1160 of SEQ ID NO: 7. In some embodiments, the C-terminal domain of SorCS2 corresponds to the region spanning from amino acid residues 1101 to 1160 of SEQ ID NO: 7.

### VII. Method for Identifying a Compound Capable of Modulating Phosphorylation of the C-terminal Cytoplasmic Domain of a Vps10p-Domain Receptor.

In one aspect, the present invention relates to a method for identifying a compound capable of modulating phosphorylation of the C-terminal cytoplasmic domain of a Vps10p-domain receptor selected from the group consisting of SorCS2, SorCS1 and SorCS3, the method comprising the steps of:
(i) providing one or more candidate compounds and a cell expressing SorCS2, SorCS1 or SorCS3;
(ii) measuring the phosphorylation level of the protein of (i) in said cell in the absence of said candidate compound;
(iii) contacting the cell of (ii) with said candidate compound;
(iv) measuring the phosphorylation level of the protein of (iii);
(v) comparing the phosphorylation levels of (ii) and (iv),
(vi) identifying the compound by electing compounds of (v) which are capable of modulating phosphorylation of the C-terminal domain of SorCS2, SorCS1, or SorCS3.

SorCS2 may be any of the four forms as set out in SEQ ID NO: 1, SEQ ID NO: 3, SEQ ID NO: 5 or SEQ ID NO: 7. Cells expressing SorCS2 may accordingly express all four forms, three forms, two forms or one form.

Cells expressing SorCS2, SorCS1, or SorCS3 may be cells from the nervous system, such as developing or adult cells of the nervous system. In some embodiments, the cell expressing SorCS2, SorCS1, or SorCS3 is a neuron cell such as a hippocampal neuron cell, a primary neuron or an iPSC (induced pluripotent stem cell) neuron. The iPSC may be obtained by methods which do not require the destruction of human embryos (19).

The cell is preferably derived from a subject with normal synaptic plasticity, such as a subject with no history of neurological disorder, neuropsychiatric disorder or mental and behavioral disorder. The subject is preferably a mammal such as a human being or a rodent such as a mouse or a rat.

Candidate Compound

In the first step of the present method, one or more candidate compounds and a cell expressing SorCS2, SorCS1, or SorCS3 are provided.

The candidate compound may be provided alone, i.e. the candidate compound is tested individually, or as part of a library or subset of a library, i.e. numerous candidate compounds are tested simultaneously or sequentially. Such libraries may be commercially available, or may be designed for the purpose of performing the present methods. Such a library may also be a fraction or a subpopulation of a commercial library.

The library may also be a library comprising a specific type of molecules, e.g. the library may be a library comprising compounds with high blood brain barrier permeability. The library may also be a library of compounds intended for central nervous system targets. The library may also comprise antibodies. In some embodiments, the candidate compound is an antibody or a functional equivalent thereof.

In some embodiments, the candidate compounds are compounds that are suitable for oral administration.

### VIII. Phosphorylation Level

The phosphorylation level of the Vps10 domain-containing receptor SorCS2, SorCS1, or SorCS3 in the cell expressing these is measured in the absence of candidate compound (step ii) or in its presence (step iv). Methods for measuring the phosphorylation level of a given protein are known in the art and include, but are not limited to, immunohistochemistry, immunocytochemistry, Western blot, SILAC, ELISA, and electrophoresis optionally combined with autoradiography.

The phosphorylation levels of SorCS2, SorCS1, or SorCS3 in the absence and in the presence of candidate compound are compared. Candidate compounds which are capable of modulating phosphorylation of the C-terminal domain of SorCS2, SorCS1, or SorCS3 are elected. In some embodiments, the phosphorylation level in the presence of the candidate compound is increased compared to the phosphorylation level in its absence. In other embodiments, the phosphorylation level in the presence of the candidate compound is decreased compared to the phosphorylation level in its absence. The compound may act as a phosphomimetic, i.e. it may mimick SorCS2, SorCS1, or SorCS3 in their phosphorylated state.

In other embodiments, the elected candidate compound, hereinafter also referred to as the compound, is capable of directly modulating phosphorylation of the C-terminal domain of the Vps10 domain-containing receptor SorCS2, SorCS1, and/or SorCS3, where the C-terminal domain of SorCS2 is as defined above.

In some embodiments, the compound is capable of modulating phosphorylation of SorCS2 at at least one of the residues 1125, 1128 and 1130 of SorCS2 as set out in SEQ ID NO: 1. Thus in one embodiment, the compound is capable of modulating phosphorylation of SorCS2 at residue 1125 of SorCS2 as set out in SEQ ID NO: 1. In another embodiment, the compound is capable of modulating phosphorylation of SorCS2 at residue 1128 of SorCS2 as set out in SEQ ID NO: 1. In yet another embodiment, the compound is capable of modulating phosphorylation of SorCS2 at residue 1130 of SorCS2 as set out in SEQ ID NO: 1. In yet another embodiment, the compound is capable of modulating phosphorylation of SorCS2 at residues 1125 and 1128 of SorCS2 as set out in SEQ ID NO: 1. In yet another embodiment, the compound is capable of modulating phosphorylation of SorCS2 at residues 1125 and 1130 of SorCS2 as set out in SEQ ID NO: 1. In yet another embodiment, the compound is capable of modulating phosphorylation of SorCS2 at residue 1128 and 1130 of SorCS2 as set out in SEQ ID NO: 1. In yet another embodiment, the compound is capable of modulating phosphorylation of SorCS2 at residue 1125, 1128 and 1130 of SorCS2 as set out in SEQ ID NO: 1. Additionally, the compound may or may not modulate phosphorylation of SorCS2 at other residues, such as residues not comprised within the C-terminal domain, wherein the C-terminal domain of SorCS2 is selected from the group consisting of:

a) amino acid residues 1099 to 1159 of SEQ ID NO: 1;

b) amino acid residues 1100 to 1159 of SEQ ID NO: 1; and

c) amino acid residues 1101 to 1159 of SEQ ID NO: 1.

In some embodiments, the compound is capable of modulating phosphorylation of SorCS2 at least one of the residues 1125, 1128 and 1130 of SorCS2 as set out in SEQ ID NO: 3. Thus in one embodiment, the compound is capable of modulating phosphorylation of SorCS2 at residue 1125 of SorCS2 as set out in SEQ ID NO: 3. In another embodiment, the compound is capable of modulating phosphorylation of SorCS2 at residue 1128 of SorCS2 as set out in SEQ ID NO: 3. In yet another embodiment, the compound is capable of modulating phosphorylation of SorCS2 at residue 1130 of SorCS2 as set out in SEQ ID NO: 3. In yet another embodiment, the compound is capable of modulating phosphorylation of SorCS2 at residues 1125 and 1128 of SorCS2 as set out in SEQ ID NO: 3. In yet another embodiment, the compound is capable of modulating phosphorylation of SorCS2 at residues 1125 and 1130 of SorCS2 as set out in SEQ ID NO: 3. In yet another embodiment, the compound is capable of modulating phosphorylation of SorCS2 at residue 1128 and 1130 of SorCS2 as set out in SEQ ID NO: 3. In yet another embodiment, the compound is capable of modulating phosphorylation of SorCS2 at residue 1125, 1128 and 1130 of SorCS2 as set out in SEQ ID NO: 3. Additionally, the compound may or may not modulate phosphorylation of SorCS2 at other residues, such as residues not comprised within the C-terminal domain, wherein the C-terminal domain of SorCS2 is selected from the group consisting of:

a) amino acid residues 1099 to 1173 of SEQ ID NO: 3;

b) amino acid residues 1100 to 1173 of SEQ ID NO: 3; and

c) amino acid residues 1101 to 1173 of SEQ ID NO: 3.

In some embodiments, the compound is capable of modulating phosphorylation of SorCS2 at at least one of the residues 1126, 1129 and 1131 of SorCS2 as set out in SEQ ID NO: 5. Thus in one embodiment, the compound is capable of modulating phosphorylation of SorCS2 at residue 1126 of SorCS2 as set out in SEQ ID NO: 5. In another embodiment, the compound is capable of modulating phosphorylation of SorCS2 at residue 1129 of SorCS2 as set out in SEQ ID NO: 5. In yet another embodiment, the compound is capable of modulating phosphorylation of SorCS2 at residue 1131 of SorCS2 as set out in SEQ ID NO: 5. In yet another embodiment, the compound is capable of modulating phosphorylation of SorCS2 at residues 1126 and 1129 of SorCS2 as set out in SEQ ID NO: 5. In yet another embodiment, the compound is capable of modulating phosphorylation of SorCS2 at residues 1126 and 1131 of SorCS2 as set out in SEQ ID NO: 5. In yet another embodiment, the compound is capable of modulating phosphorylation of SorCS2 at residues 1129 and 1131 of SorCS2 as set out in SEQ ID NO: 5. In yet another embodiment, the compound is capable of modulating phosphorylation of SorCS2 at residues 1126, 1129 and 1131 of SorCS2 as set out in SEQ ID NO: 5. Additionally, the compound may or may not modulate phosphorylation of SorCS2 at other residues, such as residues not comprised within the C-terminal domain, wherein the C-terminal domain of SorCS2 is selected from the group consisting of:

a) amino acid residues 1099 to 1174 of SEQ ID NO: 5;

b) amino acid residues 1100 to 1174 of SEQ ID NO: 5; and

c) amino acid residues 1101 to 1174 of SEQ ID NO: 5.

In some embodiments, the compound is capable of modulating phosphorylation of SorCS2 at least one of the residues 1125, 1128 and 1130 of SorCS2 as set out in SEQ ID NO: 7. Thus in one embodiment, the compound is capable of modulating phosphorylation of SorCS2 at residue 1125 of SorCS2 as set out in SEQ ID NO: 7. In another embodiment, the compound is capable of modulating phosphorylation of SorCS2 at residue 1128 of SorCS2 as set out in SEQ ID NO: 7. In yet another embodiment, the compound is capable of modulating phosphorylation of SorCS2 at residue 1130 of SorCS2 as set out in SEQ ID NO: 7. In yet another embodiment, the compound is capable of modulating phosphorylation of SorCS2 at residues 1125 and 1128 of SorCS2 as set out in SEQ ID NO: 7. In yet another embodiment, the compound is capable of modulating phosphorylation of SorCS2 at residues 1125 and 1130 of SorCS2 as set out in SEQ ID NO: 7. In yet another embodiment, the compound is capable of modulating phosphorylation of SorCS2 at residues 1128 and 1130 of SorCS2 as set out in SEQ ID NO: 7. In yet another embodiment, the compound is capable of modulating phosphorylation of SorCS2 at residues 1125, 1128 and 1130 of SorCS2 as set out in SEQ ID NO: 7. Additionally, the compound may or may not modulate phosphorylation of SorCS2 at other residues, such as residues not comprised within the C-terminal domain, wherein the C-terminal domain of SorCS2 is selected from the group consisting of:

a) amino acid residues 1099 to 1160 of SEQ ID NO: 7;

b) amino acid residues 1100 to 1160 of SEQ ID NO: 7; and

c) amino acid residues 1101 to 1160 of SEQ ID NO: 7.

The compound may modulate phosphorylation of SorCS2, SorCS1, or SorCS3 by modulating a kinase and/or a phosphatase capable of modulating phosphorylation of SorCS2, SorCS1, or SorCS3. It may additionally, or alternatively, modulate phosphorylation of SorCS2, SorCS1, or SorCS3 via modulation of BDNF-mediated signalling.

The compound may be capable of rescuing impaired BDNF activity. The compound may be capable of inducing changes in neuronal morphology.

Once elected, the compound may be further modified to increase half-life, stability, solubility, and/or bioavailability.

IX. Modulation of Expression of SorCS2 SorCS1, and/or SorCS3

Any of the compounds defined herein above may also be capable of modulating expression of any form of SorCS2 as set out in SEQ ID NO: 1, SEQ ID NO: 3, SEQ ID NO: 5 or SEQ ID NO: 7, SorCS1, or SorCS3.

Accordingly, the present invention also relates to a method for identifying a compound capable of modulating expression of SorCS2, SorCS1, and/or SorCS3, the method comprising the steps of:
(i) providing one or more candidate compounds and a cell expressing SorCS2, SorCS1 and/or SorCS3;
(ii) measuring the expression level of the protein from (i) in said cell in the absence of said candidate compound;
(iii) contacting the cell of (ii) with said candidate compound;
(iv) measuring the expression level of the protein of (iii);
(v) comparing the expression levels of (ii) and (iv),
(vi) identifying the compound by electing compounds of (v) which are capable of modulating expression of SorCS2, SorCS1 and/or SorCS3.

Expression levels of SorCS2, SorCS1, and/or SorCS3 may be determined by methods known to the skilled person, including, but not limited to, immunohistochemistry, immunocytochemistry, in situ hybridization, qPCR, rtPCR, PCR, Western blot, ELISA, SILAC and electrophoresis optionally combined with autoradiography.

In some embodiments, the compound is capable of inducing expression of SorCS2, SorCS1 and/or SorCS3. In other embodiments, the compound is capable of reducing expression of SorCS2, SorCS1 and/or SorCS3.

### EXAMPLES

### Example 1: Animal Experiments

The SorCS2 knockout mouse has been back-crossed for ten generations into C57/BL6J. Behavioral studies were done with the backcrossed homozygous mice compared to the same C57/BL6J substrain that was used for backcrossing (Glerup, Olsen et al., 2014). In the presented experiments, all mice lines had been backcrossed for ten generations into C57/BL6Jbom (Taconic) and littermate controls were used for experiments. All experiments were approved by the Danish Animal Experiments Inspectorate under the Ministry of Justice (Permit 2011/561-119) and carried according to institutional and national guidelines. All animals were bred and housed at the Animal Facility at Aarhus University. Animals were housed in groups of up to five mice per plastic cage (42×25×15 cm) under pathogen-free conditions with a 12-hour light/12-hour dark schedule and fed standard chow (Altromin #1324) and water ad libitum. Cages were cleaned every week and supplied with bedding and nesting material, a wooden stick, and a metal tunnel. Behavioral experiments were carried out using 12-16 weeks old male mice during their light cycle between 9:00 a.m. to 5:00 p.m. Each of the behavioral test described below were carried out using naïve animals tested in a randomized order by an investigator blinded to the mouse genotype. No animals were excluded from the subsequent analysis. At the end of the experiment, animals were sacrificed by cervical dislocation.

### Example 2: Marble Burying Test

Wild type and SorCS2^{-/-} mice were injected with either saline only or saline containing 20 mg/ml (20 mg/kg) fluoxetine (trade name Prozac™). Earlier studies have shown that phosphorylation of TrkB due to fluoxetine injection peaks after 1 hour (Lieto et al, Plos one 2012). Hence, the mice were left for 1 hour in their cage and then transferred to a cage where 12 marbles were placed on top of the bedding. They were then left for 20 min and hereafter the number of marbles that were more than 50% buried in each cage was counted.

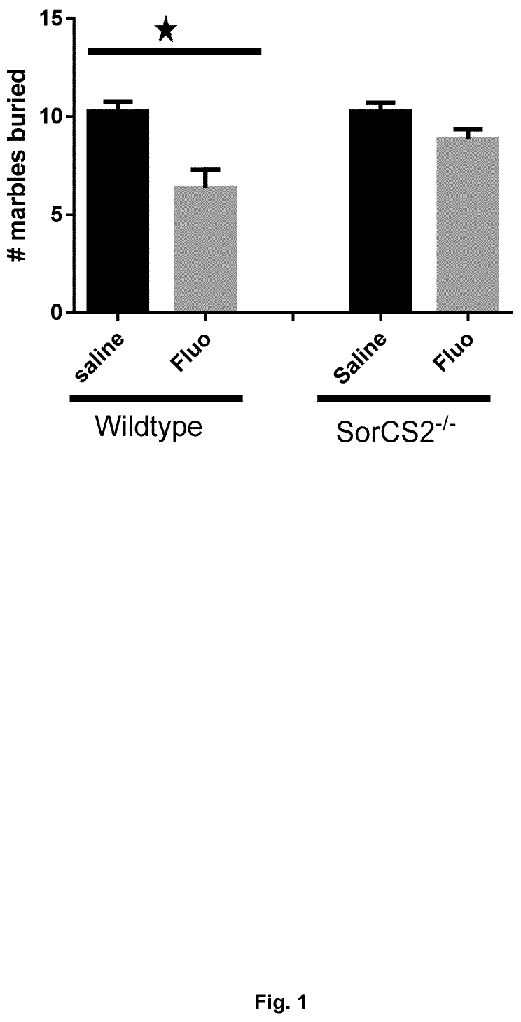

The results shown in FIG. 1 demonstrate that SorCS2^{-/-} mice buried similar levels of marbles as wild type mice. The results furthermore demonstrate that wildtype mice injected with fluoxetine were calmed and buried less marbles, while this effect was not observed in SorCS2^{-/-} mice.

### Example 3: SorCS2 is Important for BDNF-Mediated Increase in the Number of Synapses

For studies on BDNF induced formation of inhibitory synapses, neurons from p0 wild type (wt) and SorCS2^{-/-} pups were seeded at a density of 100.000 neurons per coverslip. After 7 days in vitro, the medium was changed to medium containing either 1 nM of BDNF or similar volumes of sterile D-PBS. Neurons were incubated for 72 hrs at 37° C. and 5% CO₂ before being fixed for 20 min in ice cold 4% PFA. After three 5 min washes in D-PBS, the neurons were stained against gephyrin, an inhibitory marker, and co stained against Map2, a marker of the dendritic tree. Images were taken by confocal microscopy and analysed using Imaris software.

To determine spine density in vitro, hippocampal neurons were isolated at PO and grown in culture at a density of 100.000 neurons pr coverslip. After 7 days in vitro the neurons were transfected with a plasmid containing GFP using Lipofectamine LTX reagent (15338-100) using the suppliers protocol. At 13 days in vitro the medium was changed to medium containing either 1 nM or 0 nM BDNF. Hereafter the cells were left in the incubator at 37° C. for 24 hours before being fixed in 4% PFA for 20 minutes at room temperature. To determine spine density, confocal images were obtained and subsequently assessed using Imaris software.

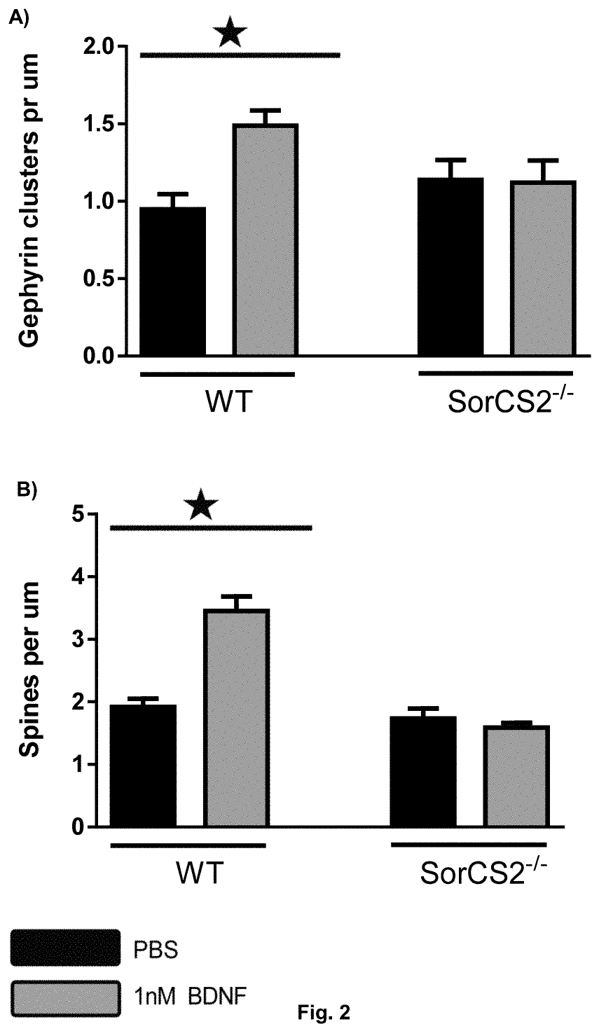

Results are shown in FIG. 2. An increase in the number of inhibitory synapses on wt neurons is observed upon stimulation with BDNF (FIG. 2A). This is not the case for neurons lacking SorCS2 (FIG. 2A). Similarly, an increase in the number of glutamatergic spines is seen in wt neurons upon stimulation with BDNF (FIG. 2B) while this is not the case for neurons lacking SorCS2 (FIG. 2B).

This example demonstrates that SorCS2 is important for BDNF-mediated increase in the number of synapses.

### Example 4: Lack of SorCS2 Affects Dendritic Complexity

To determine dendritic branching of neurons in vitro, hippocampal neurons were isolated from PO mice and grown in culture at a density of 5000 neurons per coverslip. After 24 hours the medium was changed to medium containing either 1 nM of BDNF or same volume of sterile PBS. Hereafter, the cells were left in the incubator at 37° C. for 72 hours before being fixed in 4% PFA for 20 minutes at room temperature. To determine cell morphology, the neurons were stained against β-tubulin (β-tubulin mouse mAb chemicon MAB3408). Images were taken using a confocal microscope and the branching patterns were analysed using Zen 2011 Image Processing (Carl Zeiss) as follows: The 1^{st}, 2^{nd}, 3^{rd} and 4^{th} order branching patterns the neurons were counted where 1S^{t} order branches were defined as neurites protruding from the cell soma and were only considered 1^{st} order branches if the branch was longer than the diameter of the soma. 2^{nd} order branches were defined as processes extending from the 1^{st} order branch, and so forth.

For studies on BDNF induced morphological changes of GABAergic interneurons, neurons from p3 WT and SorCS2^{-/-} pups were seeded at a density of 5.000 neurons per coverslip. The WT and SorCS2^{-/-} neurons were seeded in 1 mL Neurobasal-A Medium containing either 1 nM BDNF or similar volumes of sterile D-PBS. The neurons were incubated for 72 hrs at 37° C. and 5% CO₂ before being fixed for 20 min in ice cold 4% PFA. After three 5 min washes in D-PBS, neurons were stained, against mouse anti-β-tubulin and rabbit anti-GABA. Pictures were taken using a confocal microscope and the branching patterns were analysed using Zen 2011 Image Processing (Carl Zeiss) as follows: The 1^{st}, 2^{nd}, 3^{rd} and 4^{th} order branching patterns the neurons were counted where 1^{st} order branches were defined as neurites protruding from the cell soma and were only considered 1^{st} order branches if the branch was longer than the diameter of the soma. 2^{nd} order branches were defined as processes extending from the 1^{st} order branch, and so forth.

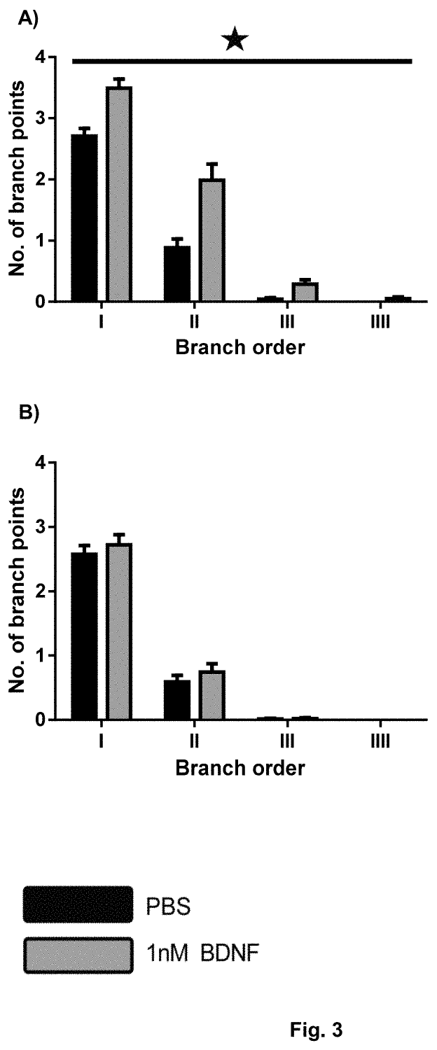

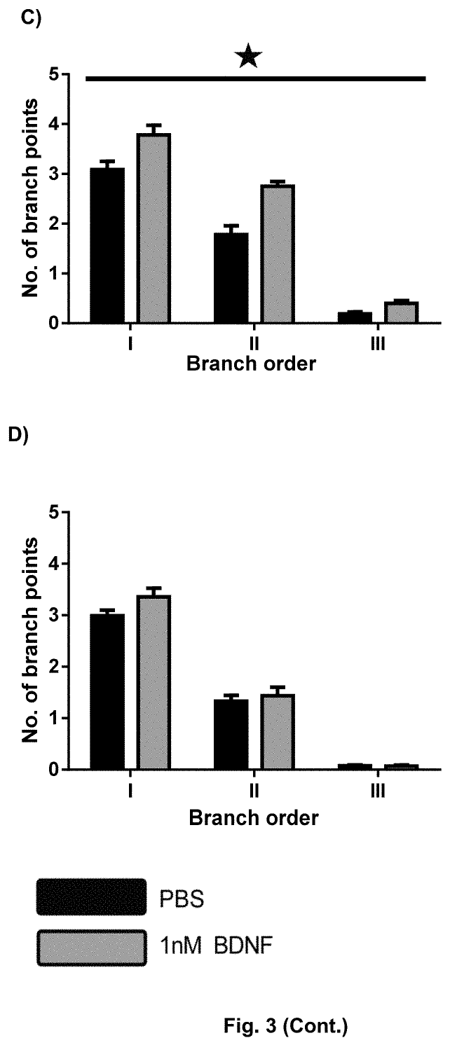

As shown in FIG. 3, upon stimulation with BDNF, wt glutamatergic neurons increase in complexity, while this is not the case for neurons lacking SorCS2 (FIG. 3A). Similarly, wt GABAergic interneurons also increase in complexity upon stimulation with BDNF while interneurons lacking SorCS2 are non-responsive (FIG. 3B).

This experiment shows that SorCS2 is required for dendritic branching.

### Example 5: SorCS2 is Important for Mediating BDNF Signalling

For studies on SorCS2 phosphorylation (FIG. 4A), wt neurons were seeded at a density of 2.5 million pr 6-well. After five days in culture, the medium was changed to phosphate-free medium+0.4 mCi/ml of ³²P labelled phosphate and left for 4 hours 37° C. and 5% CO₂. Hereafter, the neurons were washed once in phosphate-free medium and medium is added containing 1 nM BDNF or similar volume of sterile PBS. This is left on for 10 minutes before removing the media and adding lysis buffer containing protease- and phosphatase-inhibitors. From the samples, immunoprecipitation was carried out using antibodies against SorCS2. The samples were then analyzed by SDS-gel and subsequent autoradiography to evaluate the phosphorylation of SorCS2.

To study the role of SorCS2 in BDNF-mediated branching (FIG. 4B-G), SorCS2^{-/-} neurons were seeded at a density of 100.000 neurons pr coverslip in media containing the desired SorCS2 mutant together with GFP. After 6 hours, the medium was changed to normal neurobasal A medium containing either 1 nM BDNF or similar volume of sterile PBS. The neurons were incubated for 72 hrs at 37° C. and 5% CO₂ before being fixed for 20 min in ice cold 4% PFA, before a three times 5 min washes in D-PBSPictures were taken using a confocal microscope and the branching patterns were analysed using Zen 2011 Image Processing (Carl Zeiss) as follows: The 1^{st}, 2^{nd}, 3^{rd} and 4^{th} order branching patterns of GFPpositive neurons were counted where 1^{st} order branches were defined as neurites protruding from the cell soma and were only considered 1^{st} order branches if the branch was longer than the diameter of the soma. 2^{nd} order branches were defined as processes extending from the 1^{st} order branch, and so forth.

Immunoprecipitation of SorCS2 from neurons pre-incubated with radioactive phosphate showed increased phosphorylation upon BDNF-stimulation (FIG. 4A, left lane) compared to unstimulated (right lane).

Transfection of SorCS2^{-/-} neurons with a full length SorCS2 rescues the response to BDNF (FIG. 4B1). However, upon transfection with a variant lacking the intracellular domain, no increase in neurite branching is seen upon addition of BDNF (FIG. 4B2). Creating variants with a deletion of the last 21 (FIG. 4B3) or 35 (FIG. 4B4) amino acids still rescued the phenotype. However, upon deletion of 56 amino acids (FIG. 4B5) the response to BDNF was lost. By creating a full-length variant where serine residues on 3 different positions had been changed to alanine (S1125A, S1128A and S1130A), the response was still diminished (FIG. 5.2), indicating that these 3 serine residues are critical for mediating BDNF stimulus.

In addition, plasmids were created containing a serine-to-alanine substitution on either of the three serine positions of interest (1125 (FIG. 5.3), 1128 (FIGS. 5.4) and 1130 (FIG. 5.5)). When mutating serines at position 1128 and 1130, but not 1125, the response to BDNF was lost (FIG. 5), indicating an important role of these residues in mediating the response to BDNF.

In conclusion, this experiment shows that SorCS2 is important for mediating BDNF signalling.

In FIG. 6 it is shown that SorCS2 is important for phosphorylation of the BDNF-receptor TrkB.hSY5Y cells were transfected with either SorCS2, TrkB or both (FIG. 6, panels A and B). Upon stimulation with BDNF a 2.5 fold increase in phosphorylation of TrkB was seen when SorCS2 was also present, compared to when only TrkB was present (FIG. 6, panel B). Furthermore, a significant increase was also seen in phosphorylation of PLCy, a downstream kinase, when SorCS2 was present compared to when only TrkB was transfected (FIG. 6, panels C and D). This indicates a critical role of SorCS2 in mediating the response to BDNF

### Example 6: Fluoxetine Affects SorCS2 Expression Levels in the Hippocampus

3 wild-type mice were given either normal tap water or tap water containing 0.8 mg/ml fluoxetine for 3 weeks with water-chance once a week. Hereafter, the mice were euthanized by cervical dislocation and the hippocampus and cortex was dissected out. The samples were lysed in lysis buffer containing protease and phosphatase inhibitors. The samples were then analysed by Western blotting to see if treatment with fluoxetine can increase SorCS2 levels.

Western blot analysis revealed an increase in expression of SorCS2 in the hippocampus but not in the cortex (FIG. 7). The samples have been normalised to hippocampus without fluoxetine treatment.

### Example 7: Activation of SorCS2 at the Plasma Membrane Does Not Confer Increased Morphological Response and the Extracellular Domain of SorCS2 is Not Required

To study the role of the extracellular part of SorCS2 in BDNF-mediated signalling, SorCS2^{-/-} neurons were seeded at a density of 100.000 neurons pr coverslip. The neurons were transfected with construct where the extracellular part of SorCS2 had been substituted with a GFP to facilitate insertion into the membrane (denoted wt mem). Here, no difference was observed between neurons transfected with full length SorCS2 and wt mem (FIG. 8). However, neurons transfected with wt mem and stimulated with BDNF showed a significant increase in morphology compared to neurons transfected with full length SorCS2 (FIG. 8), indicating that the extracellular domain is not required for sufficient BDNF response.

To study if activation of SorCS2 at the plasmamembrane was sufficient to induce a morphological response in neurons lacking SorCS2, SorCS2^{-/-} neurons were seeded at a density of 100.000 neurons per coverslip. The neurons were transfected with construct where the extracellular part of SorCS2 had been substituted with a GFP to facilitate insertion into the membrane. Furthermore, serines described earlier at positions 1125, 1128 and 1130 had been changed to aspartic acid to mimic phosphorylated serines (denoted mut mem). A significantly reduced morphology was observed in neurons transfected with mut mem compared to neurons transfected with full length SorCS2 (FIG. 9). This reduction was rescued upon addition of BDNF (FIG. 9), indicating that activation of SorCS2 at the membrane does not confer increased morphology in neurons.

### Example 8: Activation of the SorCS2 Tail is Sufficient to Induce a Response Similar to the Addition of BDNF

To study if a soluble variant of the SorCS2 tail would be sufficient to induce a morphological response, SorCS2^{-/-} neurons were seeded at a density of 100.000 neurons per coverslip. These neurons were transfected with a construct containing the intracellular part of SorCS2 (FIG. 10.1), denoted “wt soluble tail”. Neurons transfected with wt soluble tail showed an increased morphology compared to neurons transfected with full length SorCS2. This response was further increased upon addition of BDNF. We next created a construct containing the intracellular part of SorCS2 where serines at positions 1125, 1128 and 1130 had been changed to aspartic acids to mimic phosphorylated serines (FIG. 10.2), denoted “Activated soluble tail”. A significant response was observed when comparing neurons transfected with Active soluble tail compared with neurons transfected with full length SorCS2. This response was not further increased when adding BDNF to Active soluble tail. This shows that Active soluble tail is sufficient to induce a morphological response in neurons. In summary, SorCS2 is not only critical for BDNF mediated response in neurons, but also sufficient.

### Example 9: Constitutive Active SorCS2 Tail Induces a Response which is not Further Increased Upon Addition of BDNF

We next created peptides similar to the intracellular tail of SorCS2. The first peptide had similar sequence to wild type SorCS2 tail except the serines at position 1125, 1128 and 1130, which had been changed to aspartic acids. Furthermore a TAT sequence had been added to facilitate entry into the neurons (FIG. 11), denoted “Ppep”. To study if this peptide would be able to induce a neurotrophic response, wild type neurons, expressing endogenous levels of SorCS2, were seeded at a density of 20.000 neurons per coverslip in media containing either 0.1, 1 or 10 μM in combination with either 1 nM BDNF or similar volume of sterile saline. While neither 0.1 nor 10 μM did elicit a response, 1 μM of Ppep induced a response that was significant compared to control (no stimulation) (FIG. 11.4). In addition, no further increase was observed upon BDNF treatment (FIG. 11.2), indicating that Ppep is sufficient to induce a neurotrophic response in neurons.

### Example 10: Constitutively Inactive SorCS2 Tail is Able to Inhibit BDNF Induced Morphological Response, Even in the Presence of BDNF

Since addition of pPep was sufficient to induce a neurotrophic response, even in the absence of BDNF, we wanted to investigate if addition of an inactive peptide was sufficient to inhibit BDNF signalling, even in the presence of BDNF. We therefore created a peptide where serines at positions 1125, 1128 and 1130 of the SorCS2 tail had been changed to alanines to mimic an inactive SorCS2 tail (denoted Apep in FIG. 12). Furthermore, the peptide constitutes a TAT-sequence (a HIV protein transduction domain) to facilitate entry into the cell. At 0.1 μM of Apep BDNF was still able to induce a significant response (FIG. 12.1). However, this was not the case when applying 1 μM Apep (FIG. 12.2) or 10 μM (FIG. 12.3). When compared to control, no difference was observed between a control (where no BDNF had been added) and 1 μM Apep +BDNF (FIG. 12.4). This indicates that at 1 μM, Apep is able to inhibit BDNF induced morphological response, even in the presence of BDNF.

### Example 11: Short Peptides are Able to Elicit both Activation and Inhibition of Neurotrophic Response

Smaller peptides were created to study if these would be sufficient to induce a neurotrophic response. When adding 1 nM BDNF (FIG. 13.2), wt neurons increased in complexity compared to WT neurons without BDNF (FIG. 13.1). As seen in Examples 9 & 10, a peptide where serines in positions 25, 28 and 30 had been mutated to alanines inhibits morphological response, even in the presence of BDNF (FIG. 13.3), and a peptide where serines in positions 25, 28 and 30 had been changed to aspartic acids was able to induce a neurotrophic response, even in the absence of BDNF (FIG. 13.4). We therefore created smaller peptides where serines had been changed to alanines. This peptide was able to inhibit the neurotrophic response induced by BDNF (FIG. 13.5). Furthermore, a short peptide where serines had been changed to aspartic acids was able to induce a neurotrophic response, even in the absence of BDNF (FIG. 13.6)

### Example 12: Sequence Alignment of the Intracellular Tails of SorCS1, SorCS2 and SorCS3 Reveal Highly Conserved Sortilin Family Intracellular Tails

Besides a general highly conserved sequence, serines at positions 1125, 1128 and 1130 are conserved in SorCS1, as shown in FIG. 14. In SorCS3, serines at positions 1128 and 1130 are conserved, (these serines were found to be the two important serines in mediating BDNF-response, FIG. 5). The tails of SorCS1 and SorCS3 may therefore also mediate neurotrophic signalling similarly to SorCS2.

### Example 13: Excessive Activation of TrkB is Linked to Temporal Lope Epilepsy (TLE)

Excessive activation of TrkB caused by status epilepticus promotes development of temporal lope epilepsy (TLE)(3). Global inhibition of TrkB leads to decreased neuronal survival and is therefore not desirable (3). To test if lead compound is able prevent unwanted effects of excessive TrkB signalling caused by status epilepticus, without decreasing neuronal survival, we will inject lead compound prior to injection of kainic acid as similar to described in (3). Here we will test if lead compound is able to prevent development of epileptic seizures and/or able to prevent the unwanted effects caused by excessive activation of TrkB, and thereby able to prevent development of temporal lope epilepsy.

### Example 14: Loss of BDNF Signalling is Associated with Huntington's Disease

Huntington's disease is an autosomal dominant neurodegenerative disorder believed to be caused by lack of trophic support to neurons due to lost BDNF signalling. (4). To study if a lead compound is able to activate downstream signalling cascades in Huntington's disease, we use a transgenic mouse model of Huntington's disease named BACHD where the full length human mutant huntingtin has been inserted. In these mice, BDNF is not able to induce synaptic strengthening or LTP (4). Using electrophysiology we investigate if addition of lead compound is able to induce LTP in BACHD mice brains.

### Example 15: BDNF is Implicated in Alzheimer's Disease

BDNF is considered critical for trophic support on neurons in the central nervous system, and reduced levels of BDNF observed in patients suffering from Alzheimer's disease has indicated a possible role in the disease(5). Here, BDNF may play a role in preventing formation of amyloid plaques, considered to be the pathological hallmark of Alzheimer's disease. To study if lead compound is able to attenuate the development of the disease, hippocampal neurons will be seeded at a density of 100.00 neurons pr coverslip. Lead compound till be added 4 hours prior to addition of the toxic component of the amyloid plaques Aβ₁₋₄₂ as described in (6). Addition of Aβ₁₋₄₂ has previously been described to lead to neuronal death, and we will therefore study the neuronal survival when adding Aβ₁₋₄₂ along with our lead compound or vehicle.

### Example 16: BDNF Haploinsufficiency is Associated with WAGR Syndrome

In article (7), the authors examine a group of children suffering from WAGR syndrome, a disease where part of the chromosome 11p13 is deleted. In a subgroup of children, the bdnf gene is also deleted leading to BDNF haploinsufficiency. By the age of 10, 100% of the children with bdnf deletion suffered from obesity. Here, it is believed that BDNF acts within the hypothalamus to regulate energy uptake. Mice which are haploinsufficient for BDNF are also obese (8), something which can be rescued by intracerebroventricular BDNF injection (9). Mice with a mutation in the leptin receptor, an important receptor for energy homeostasis (db/db mice), and mice haploinsufficient for BDNF are both obese compared to wild type animals. In db/db mice, central or peripheral administration of BDNF decreases food intake and increases energy expenditure and prevented diabetes (10, 11). To study if lead compound is able to attenuate the development of diabetes, we study db/db mice and mice haploinsufficient for BDNF. Here, lead compound or vehicle are injected in 4 weeks old db/db mice, mice haploinsufficient for BDNF or wild type mice mice for 12 weeks. During this course, blood samples will be acquired twice a week to measure glucose levels and insulin levels. Furthermore, the body weight, energy expenditure and food intake of the animals will be measured throughout the experiment.

Db/db mice are considered prediabetic around 8 weeks of age. We therefore want to study if administration of lead compound is able to attenuate the development of diabetes in db/db mice. Here, the lead compound or vehicle will be injected twice a week in 8 weeks old db/db mice, mice haploinsufficient for BDNF or wild type mice for 8 weeks. Here, food intake, energy expenditure, body weight and insulin and glucose levels will be measured throughout this period. Hereafter, food will be removed over night to measure fasting levels of glucose.

### Example 17: Detection of Phosphorylated SorCS2

Wildtype postnatal day 0 pups are euthanized by decapitation, brains removed and hippocampi dissected into ice cold Leibovitz's L15 medium. After dissection, the tissue is dissociated for 30 minutes in 20 U/ml pre-activated papain. Hereafter, the tissue is washed in DMEM containing 0.01 mg/ml DNase and 10% Fetal Bovine Serum (FBS), before being triturated in DMEM containing 0.01 mg/ml DNase and 10% FBS.

Hereafter, DMEM is removed and Neurobasal-A medium is added to the cells. The cells are seeded on Poly-D and laminin coverslips at a density of 100.000 neurons per coverslip and left at 37° C. and 5% CO₂, with medium change every second day. After five days in culture, the medium is changed to phosphate-free medium+0.4 mCi/ml of P32 labelled phosphate and left for 4 hours 37° C. and 5% CO₂. Hereafter, the neurons are washed once in phosphate-free medium and medium is added containing one unit from a drug library. Such compounds would be found from compound libraries such as, but not exclusively, a CNS compound library from OTAVA chemicals (http://www.otavachemicals.com/products/compound-libraries-for-hts/cns-library) or Kinase directed libraries from ChemBridge (http://www.chembridge.com/screening_libraries/targeted_libraries/). These libraries are designed to have high blood-brain barrier permeability.

Neurons and medium are left on for 10 minutes before removing the media and adding lysis buffer containing protease- and phosphatase-inhibitors. From the samples, immunoprecipitation will be carried out using antibodies against SorCS2. The samples are then analyzed by SDS-gel and subsequent autoradiography to evaluate the phosphorylation of SorCS2. As a control, simultaneous experiments are carried out in neurons lacking SorCS2 expression.

### Example 18: SorCS2 Expression Analysis

Wt mice are given the drug, either by injection or though water, for 3 weeks. Hereafter the mice are euthanized by cervical dislocation and areas of interest are dissected from the mouse. These areas are then analyzed by western blot or ELISA to study regulation of several genes, including SorCS2.

### Example 19: Screening for Small Molecules Increasing SorCS2 Phoshorylation in Neurons

A screening of CNS compound libraries for their ability to induce SorCS2 phosphorylation or increase SorCS2 expression in primary hippocampal neurons is performed. Candidate compounds are tested further for their ability to induce neurite outgrowth in a SorCS2-dependent manner using WT and KO hippocampal neurons. A lead compound is identified and be tested for its ability to modulate neuropsychiatric behavior in an animal model e.g. BDNF heterozygous mice.

Sequences

SEQ ID NO. 1: SorCS2 protein sequence with tail A; Homo sapiens.
MAHRGPSRASKGPGPTARAPSPGAPPPPRSPRSRPLLLLLLLLGACGAAGRSPEPG
RLGPHAQLTRVPRSPPAGRAEPGGGEDRQARGTEPGAPGPSPGPAPGPGEDGAPA
AGYRRWERAAPLAGVASRAQVSLISTSFVLKGDATHNQAMVHWTGENSSVILILTKYY
HADMGKVLESSLWRSSDFGTSYTKLTLQPGVTTVIDNFYICPTNKRKVILVSSSLSDRD
QSLFLSADEGATFQKQPIPFFVETLIFHPKEEDKVLAYTKESKLYVSSDLGKKWTLLQE
RVTKDHVFWSVSGVDADPDLVHVEAQDLGGDFRYVTCAIHNCSEKMLTAPFAGPIDH
GSLTVQDDYIFFKATSANQTKYYVSYRRNEFVLMKLPKYALPKDLQIISTDESQVFVAV
QEWYQMDTYNLYQSDPRGVRYALVLQDVRSSRQAEESVLIDILEVRGVKGVFLANQK
IDGKVMTLITYNKGRDWDYLRPPSMDMNGKPTNCKPPDCHLHLHLRWADNPYVSGT
VHTKDTAPGLIMGAGNLGSQLVEYKEEMYITSDCGHTWRQVFEEEHHILYLDHGGVIV
AIKDTSIPLKILKFSVDEGLTWSTHNFTSTSVFVDGLLSEPGDETLVMTVFGHISFRSD
WELVKVDFRPSFSRQCGEEDYSSWELSNLQGDRCIMGQQRSFRKRKSTSWCIKGR
SFTSALTSRVCECRDSDFLCDYGFERSSSSESSTNKCSANFWFNPLSPPDDCALGQT
YTSSLGYRKVVSNVCEGGVDMQQSQVQLQCPLTPPRGLQVSIQGEAVAVRPGEDVL
FVVRQEQGDVLTTKYQVDLGDGFKAMYVNLTLTGEPIRHRYESPGIYRVSVRAENTA
GHDEAVLFVQVNSPLQALYLEVVPVIGLNQEVNLTAVLLPLNPNLTVFYWWIGHSLQP
LLSLDNSVTTRFSDTGDVRVTVQAACGNSVLQDSRVLRVLDQFQVMPLQFSKELDAY
NPNTPEWREDVGLVVTRLLSKETSVPQELLVTVVKPGLPTLADLYVLLPPPRPTRKRS
LSSDKRLAAIQQVLNAQKISFLLRGGVRVLVALRDTGTGAEQLGGGGGYWAVVVLFVI
GLFAAGAFILYKFKRKRPGRTVYAQMHNEKEQEMTSPVSHSEDVQGAVQGNHSGVV
LSINSREMHSYLVS
SEQ ID NO. 2: SorCS2 protein sequence with tail A; S1125X, S1128X,
S1130X; X is E, D; or phosphoserine (J).
MAHRGPSRASKGPGPTARAPSPGAPPPPRSPRSRPLLLLLLLLGACGAAGRSPEPG
RLGPHAQLTRVPRSPPAGRAEPGGGEDRQARGTEPGAPGPSPGPAPGPGEDGAPA
AGYRRWERAAPLAGVASRAQVSLISTSFVLKGDATHNQAMVHWTGENSSVILILTKYY
HADMGKVLESSLWRSSDFGTSYTKLTLQPGVTTVIDNFYICPTNKRKVILVSSSLSDRD
QSLFLSADEGATFQKQPIPFFVETLIFHPKEEDKVLAYTKESKLYVSSDLGKKWTLLQE
RVTKDHVFWSVSGVDADPDLVHVEAQDLGGDFRYVTCAIHNCSEKMLTAPFAGPIDH
GSLTVQDDYIFFKATSANQTKYYVSYRRNEFVLMKLPKYALPKDLQIISTDESQVFVAV
QEWYQMDTYNLYQSDPRGVRYALVLQDVRSSRQAEESVLIDILEVRGVKGVFLANQK
IDGKVMTLITYNKGRDWDYLRPPSMDMNGKPTNCKPPDCHLHLHLRWADNPYVSGT
VHTKDTAPGLIMGAGNLGSQLVEYKEEMYITSDCGHTWRQVFEEEHHILYLDHGGVIV
AIKDTSIPLKILKFSVDEGLTWSTHNFTSTSVFVDGLLSEPGDETLVMTVFGHISFRSD
WELVKVDFRPSFSRQCGEEDYSSWELSNLQGDRCIMGQQRSFRKRKSTSWCIKGR
SFTSALTSRVCECRDSDFLCDYGFERSSSSESSTNKCSANFWFNPLSPPDDCALGQT
YTSSLGYRKVVSNVCEGGVDMQQSQVQLQCPLTPPRGLQVSIQGEAVAVRPGEDVL
FVVRQEQGDVLTTKYQVDLGDGFKAMYVNLTLTGEPIRHRYESPGIYRVSVRAENTA
GHDEAVLFVQVNSPLQALYLEVVPVIGLNQEVNLTAVLLPLNPNLTVFYWWIGHSLQP
LLSLDNSVTTRFSDTGDVRVTVQAACGNSVLQDSRVLRVLDQFQVMPLQFSKELDAY
NPNTPEWREDVGLVVTRLLSKETSVPQELLVTVVKPGLPTLADLYVLLPPPRPTRKRS
LSSDKRLAAIQQVLNAQKISFLLRGGVRVLVALRDTGTGAEQLGGGGGYWAVVVLFVI
GLFAAGAFILYKFKRKRPGRTVYAQMHNEKEQEMTXPVXHXEDVQGAVQGNHSGVV
LSINSREMHSYLVS
SEQ ID NO. 3: SorCS2 protein sequence with tail B; Homo sapiens.
MAHRGPSRASKGPGPTARAPSPGAPPPPRSPRSRPLLLLLLLLGACGAAGRSPEPG
RLGPHAQLTRVPRSPPAGRAEPGGGEDRQARGTEPGAPGPSPGPAPGPGEDGAPA
AGYRRWERAAPLAGVASRAQVSLISTSFVLKGDATHNQAMVHWTGENSSVILILTKYY
HADMGKVLESSLWRSSDFGTSYTKLTLQPGVTTVIDNFYICPTNKRKVILVSSSLSDRD
QSLFLSADEGATFQKQPIPFFVETLIFHPKEEDKVLAYTKESKLYVSSDLGKKWTLLQE
RVTKDHVFWSVSGVDADPDLVHVEAQDLGGDFRYVTCAIHNCSEKMLTAPFAGPIDH
GSLTVQDDYIFFKATSANQTKYYVSYRRNEFVLMKLPKYALPKDLQIISTDESQVFVAV
QEWYQMDTYNLYQSDPRGVRYALVLQDVRSSRQAEESVLIDILEVRGVKGVFLANQK
IDGKVMTLITYNKGRDWDYLRPPSMDMNGKPTNCKPPDCHLHLHLRWADNPYVSGT
VHTKDTAPGLIMGAGNLGSQLVEYKEEMYITSDCGHTWRQVFEEEHHILYLDHGGVIV
AIKDTSIPLKILKFSVDEGLTWSTHNFTSTSVFVDGLLSEPGDETLVMTVFGHISFRSD
WELVKVDFRPSFSRQCGEEDYSSWELSNLQGDRCIMGQQRSFRKRKSTSWCIKGR
SFTSALTSRVCECRDSDFLCDYGFERSSSSESSTNKCSANFWFNPLSPPDDCALGQT
YTSSLGYRKVVSNVCEGGVDMQQSQVQLQCPLTPPRGLQVSIQGEAVAVRPGEDVL
FVVRQEQGDVLTTKYQVDLGDGFKAMYVNLTLTGEPIRHRYESPGIYRVSVRAENTA
GHDEAVLFVQVNSPLQALYLEVVPVIGLNQEVNLTAVLLPLNPNLTVFYWWIGHSLQP
LLSLDNSVTTRFSDTGDVRVTVQAACGNSVLQDSRVLRVLDQFQVMPLQFSKELDAY
NPNTPEWREDVGLVVTRLLSKETSVPQELLVTVVKPGLPTLADLYVLLPPPRPTRKRS
LSSDKRLAAIQQVLNAQKISFLLRGGVRVLVALRDTGTGAEQLGGGGGYWAVVVLFVI
GLFAAGAFILYKFKRKRPGRTVYAQMHNEKEQEMTSPVSHSEDVQGAVQEEFIDDDL
DSQTLGGNHSGVVLSINSREMHSYLVS
SEQ ID NO. 4: SorCS2 protein sequence with tail B; S1125X, S1128X,
S1130X; X is E, D; or phosphoserine (J).
MAHRGPSRASKGPGPTARAPSPGAPPPPRSPRSRPLLLLLLLLGACGAAGRSPEPG
RLGPHAQLTRVPRSPPAGRAEPGGGEDRQARGTEPGAPGPSPGPAPGPGEDGAPA
AGYRRWERAAPLAGVASRAQVSLISTSFVLKGDATHNQAMVHWTGENSSVILILTKYY
HADMGKVLESSLWRSSDFGTSYTKLTLQPGVTTVIDNFYICPTNKRKVILVSSSLSDRD
QSLFLSADEGATFQKQPIPFFVETLIFHPKEEDKVLAYTKESKLYVSSDLGKKWTLLQE
RVTKDHVFWSVSGVDADPDLVHVEAQDLGGDFRYVTCAIHNCSEKMLTAPFAGPIDH
GSLTVQDDYIFFKATSANQTKYYVSYRRNEFVLMKLPKYALPKDLQIISTDESQVFVAV
QEWYQMDTYNLYQSDPRGVRYALVLQDVRSSRQAEESVLIDILEVRGVKGVFLANQK
IDGKVMTLITYNKGRDWDYLRPPSMDMNGKPTNCKPPDCHLHLHLRWADNPYVSGT
VHTKDTAPGLIMGAGNLGSQLVEYKEEMYITSDCGHTWRQVFEEEHHILYLDHGGVIV
AIKDTSIPLKILKFSVDEGLTWSTHNFTSTSVFVDGLLSEPGDETLVMTVFGHISFRSD
WELVKVDFRPSFSRQCGEEDYSSWELSNLQGDRCIMGQQRSFRKRKSTSWCIKGR
SFTSALTSRVCECRDSDFLCDYGFERSSSSESSTNKCSANFWFNPLSPPDDCALGQT
YTSSLGYRKVVSNVCEGGVDMQQSQVQLQCPLTPPRGLQVSIQGEAVAVRPGEDVL
FVVRQEQGDVLTTKYQVDLGDGFKAMYVNLTLTGEPIRHRYESPGIYRVSVRAENTA
GHDEAVLFVQVNSPLQALYLEVVPVIGLNQEVNLTAVLLPLNPNLTVFYWWIGHSLQP
LLSLDNSVTTRFSDTGDVRVTVQAACGNSVLQDSRVLRVLDQFQVMPLQFSKELDAY
NPNTPEWREDVGLVVTRLLSKETSVPQELLVTVVKPGLPTLADLYVLLPPPRPTRKRS
LSSDKRLAAIQQVLNAQKISFLLRGGVRVLVALRDTGTGAEQLGGGGGYWAVVVLFVI
GLFAAGAFILYKFKRKRPGRTVYAQMHNEKEQEMTXPVXHXEDQGAVQEEFIDDDLD
SQTLGGNHSGVVLSINSREMHSYLVS
SEQ ID NO. 5: SorCS2 protein sequence with tail C; Homo sapiens.
MAHRGPSRASKGPGPTARAPSPGAPPPPRSPRSRPLLLLLLLLGACGAAGRSPEPG
RLGPHAQLTRVPRSPPAGRAEPGGGEDRQARGTEPGAPGPSPGPAPGPGEDGAPA
AGYRRWERAAPLAGVASRAQVSLISTSFVLKGDATHNQAMVHWTGENSSVILILTKYY
HADMGKVLESSLWRSSDFGTSYTKLTLQPGVTTVIDNFYICPTNKRKVILVSSSLSDRD
QSLFLSADEGATFQKQPIPFFVETLIFHPKEEDKVLAYTKESKLYVSSDLGKKWTLLQE
RVTKDHVFWSVSGVDADPDLVHVEAQDLGGDFRYVTCAIHNCSEKMLTAPFAGPIDH
GSLTVQDDYIFFKATSANQTKYYVSYRRNEFVLMKLPKYALPKDLQIISTDESQVFVAV
QEWYQMDTYNLYQSDPRGVRYALVLQDVRSSRQAEESVLIDILEVRGVKGVFLANQK
IDGKVMTLITYNKGRDWDYLRPPSMDMNGKPTNCKPPDCHLHLHLRWADNPYVSGT
VHTKDTAPGLIMGAGNLGSQLVEYKEEMYITSDCGHTWRQVFEEEHHILYLDHGGVIV
AIKDTSIPLKILKFSVDEGLTWSTHNFTSTSVFVDGLLSEPGDETLVMTVFGHISFRSD
WELVKVDFRPSFSRQCGEEDYSSWELSNLQGDRCIMGQQRSFRKRKSTSWCIKGR
SFTSALTSRVCECRDSDFLCDYGFERSSSSESSTNKCSANFWFNPLSPPDDCALGQT
YTSSLGYRKVVSNVCEGGVDMQQSQVQLQCPLTPPRGLQVSIQGEAVAVRPGEDVL
FVVRQEQGDVLTTKYQVDLGDGFKAMYVNLTLTGEPIRHRYESPGIYRVSVRAENTA
GHDEAVLFVQVNSPLQALYLEVVPVIGLNQEVNLTAVLLPLNPNLTVFYWWIGHSLQP
LLSLDNSVTTRFSDTGDVRVTVQAACGNSVLQDSRVLRVLDQFQVMPLQFSKELDAY
NPNTPEWREDVGLVVTRLLSKETSVPQELLVTVVKPGLPTLADLYVLLPPPRPTRKRS
LSSDKRLAAIQQVLNAQKISFLLRGGVRVLVALRDTGTGAEQLGGGGGYWAVVVLFVI
GLFAAGAFILYKFKSRKRPGRTVYAQMHNEKEQEMTSPVSHSEDVQGAVQEEFIDDD
LDSQTLGGNHSGVVLSINSREMHSYLVS
SEQ ID NO. 6: SorCS2 protein sequence with tail C; S1125X, S1128X,
S1130X; X is E, D; or phosphoserine (J).
MAHRGPSRASKGPGPTARAPSPGAPPPPRSPRSRPLLLLLLLLGACGAAGRSPEPG
RLGPHAQLTRVPRSPPAGRAEPGGGEDRQARGTEPGAPGPSPGPAPGPGEDGAPA
AGYRRWERAAPLAGVASRAQVSLISTSFVLKGDATHNQAMVHWTGENSSVILILTKYY
HADMGKVLESSLWRSSDFGTSYTKLTLQPGVTTVIDNFYICPTNKRKVILVSSSLSDRD
QSLFLSADEGATFQKQPIPFFVETLIFHPKEEDKVLAYTKESKLYVSSDLGKKWTLLQE
RVTKDHVFWSVSGVDADPDLVHVEAQDLGGDFRYVTCAIHNCSEKMLTAPFAGPIDH
GSLTVQDDYIFFKATSANQTKYYVSYRRNEFVLMKLPKYALPKDLQIISTDESQVFVAV
QEWYQMDTYNLYQSDPRGVRYALVLQDVRSSRQAEESVLIDILEVRGVKGVFLANQK
IDGKVMTLITYNKGRDWDYLRPPSMDMNGKPTNCKPPDCHLHLHLRWADNPYVSGT
VHTKDTAPGLIMGAGNLGSQLVEYKEEMYITSDCGHTWRQVFEEEHHILYLDHGGVIV
AIKDTSIPLKILKFSVDEGLTWSTHNFTSTSVFVDGLLSEPGDETLVMTVFGHISFRSD
WELVKVDFRPSFSRQCGEEDYSSWELSNLQGDRCIMGQQRSFRKRKSTSWCIKGR
SFTSALTSRVCECRDSDFLCDYGFERSSSSESSTNKCSANFWFNPLSPPDDCALGQT
YTSSLGYRKVVSNVCEGGVDMQQSQVQLQCPLTPPRGLQVSIQGEAVAVRPGEDVL
FVVRQEQGDVLTTKYQVDLGDGFKAMYVNLTLTGEPIRHRYESPGIYRVSVRAENTA
GHDEAVLFVQVNSPLQALYLEVVPVIGLNQEVNLTAVLLPLNPNLTVFYWWIGHSLQP
LLSLDNSVTTRFSDTGDVRVTVQAACGNSVLQDSRVLRVLDQFQVMPLQFSKELDAY
NPNTPEWREDVGLVVTRLLSKETSVPQELLVTVVKPGLPTLADLYVLLPPPRPTRKRS
LSSDKRLAAIQQVLNAQKISFLLRGGVRVLVALRDTGTGAEQLGGGGGYWAVVVLFVI
GLFAAGAFILYKFKSRKRPGRTVYAQMHNEKEQEMTXPVXHXEDQGAVQEEFIDDDL
DSQTLGGNHSGVVLSINSREMHSYLVS
SEQ ID NO. 7: SorCS2 protein sequence with tail D; Homo sapiens.
MAHRGPSRASKGPGPTARAPSPGAPPPPRSPRSRPLLLLLLLLGACGAAGRSPEPG
RLGPHAQLTRVPRSPPAGRAEPGGGEDRQARGTEPGAPGPSPGPAPGPGEDGAPA
AGYRRWERAAPLAGVASRAQVSLISTSFVLKGDATHNQAMVHWTGENSSVILILTKYY
HADMGKVLESSLWRSSDFGTSYTKLTLQPGVTTVIDNFYICPTNKRKVILVSSSLSDRD
QSLFLSADEGATFQKQPIPFFVETLIFHPKEEDKVLAYTKESKLYVSSDLGKKWTLLQE
RVTKDHVFWSVSGVDADPDLVHVEAQDLGGDFRYVTCAIHNCSEKMLTAPFAGPIDH
GSLTVQDDYIFFKATSANQTKYYVSYRRNEFVLMKLPKYALPKDLQIISTDESQVFVAV
QEWYQMDTYNLYQSDPRGVRYALVLQDVRSSRQAEESVLIDILEVRGVKGVFLANQK
IDGKVMTLITYNKGRDWDYLRPPSMDMNGKPTNCKPPDCHLHLHLRWADNPYVSGT
VHTKDTAPGLIMGAGNLGSQLVEYKEEMYITSDCGHTWRQVFEEEHHILYLDHGGVIV
AIKDTSIPLKILKFSVDEGLTWSTHNFTSTSVFVDGLLSEPGDETLVMTVFGHISFRSD
WELVKVDFRPSFSRQCGEEDYSSWELSNLQGDRCIMGQQRSFRKRKSTSWCIKGR
SFTSALTSRVCECRDSDFLCDYGFERSSSSESSTNKCSANFWFNPLSPPDDCALGQT
YTSSLGYRKVVSNVCEGGVDMQQSQVQLQCPLTPPRGLQVSIQGEAVAVRPGEDVL
FVVRQEQGDVLTTKYQVDLGDGFKAMYVNLTLTGEPIRHRYESPGIYRVSVRAENTA
GHDEAVLFVQVNSPLQALYLEVVPVIGLNQEVNLTAVLLPLNPNLTVFYWWIGHSLQP
LLSLDNSVTTRFSDTGDVRVTVQAACGNSVLQDSRVLRVLDQFQVMPLQFSKELDAY
NPNTPEWREDVGLVVTRLLSKETSVPQELLVTVVKPGLPTLADLYVLLPPPRPTRKRS
LSSDKRLAAIQQVLNAQKISFLLRGGVRVLVALRDTGTGAEQLGGGGGYWAVVVLFVI
GLFAAGAFILYKFKSRKRPGRTVYAQMHNEKEQEMTSPVSHSEDVQGAVQGNHSGV
VLSINSREMHSYLVS
SEQ ID NO. 8: SorCS2 protein sequence with tail D; S1125X, S1128X,
S1130X; X is E, D; or phosphoserine (J).
MAHRGPSRASKGPGPTARAPSPGAPPPPRSPRSRPLLLLLLLLGACGAAGRSPEPG
RLGPHAQLTRVPRSPPAGRAEPGGGEDRQARGTEPGAPGPSPGPAPGPGEDGAPA
AGYRRWERAAPLAGVASRAQVSLISTSFVLKGDATHNQAMVHWTGENSSVILILTKYY
HADMGKVLESSLWRSSDFGTSYTKLTLQPGVTTVIDNFYICPTNKRKVILVSSSLSDRD
QSLFLSADEGATFQKQPIPFFVETLIFHPKEEDKVLAYTKESKLYVSSDLGKKWTLLQE
RVTKDHVFWSVSGVDADPDLVHVEAQDLGGDFRYVTCAIHNCSEKMLTAPFAGPIDH
GSLTVQDDYIFFKATSANQTKYYVSYRRNEFVLMKLPKYALPKDLQIISTDESQVFVAV
QEWYQMDTYNLYQSDPRGVRYALVLQDVRSSRQAEESVLIDILEVRGVKGVFLANQK
IDGKVMTLITYNKGRDWDYLRPPSMDMNGKPTNCKPPDCHLHLHLRWADNPYVSGT
VHTKDTAPGLIMGAGNLGSQLVEYKEEMYITSDCGHTWRQVFEEEHHILYLDHGGVIV
AlKDTSIPLKILKFSVDEGLTWSTHNFTSTSVFVDGLLSEPGDETLVMTVFGHISFRSD
WELVKVDFRPSFSRQCGEEDYSSWELSNLQGDRCIMGQQRSFRKRKSTSWCIKGR
SFTSALTSRVCECRDSDFLCDYGFERSSSSESSTNKCSANFWFNPLSPPDDCALGQT
YTSSLGYRKVVSNVCEGGVDMQQSQVQLQCPLTPPRGLQVSIQGEAVAVRPGEDVL
FVVRQEQGDVLTTKYQVDLGDGFKAMYVNLTLTGEPIRHRYESPGIYRVSVRAENTA
GHDEAVLFVQVNSPLQALYLEVVPVIGLNQEVNLTAVLLPLNPNLTVFYWWIGHSLQP
LLSLDNSVTTRFSDTGDVRVTVQAACGNSVLQDSRVLRVLDQFQVMPLQFSKELDAY
NPNTPEWREDVGLVVTRLLSKETSVPQELLVTVVKPGLPTLADLYVLLPPPRPTRKRS
LSSDKRLAAIQQVLNAQKISFLLRGGVRVLVALRDTGTGAEQLGGGGGYWAVVVLFVI
GLFAAGAFILYKFKSRKRPGRTVYAQMHNEKEQEMTXPVXHXEDQGAVQGNHSGVV
LSINSREMHSYLVS
SEQ ID NO. 9: Truncated Vps10p-domain receptor tail
X₁X₂X₃VX₄X₅X₆E
SEQ ID NO. 10: Truncated Vps10p-domain receptor tail
KEQEMX₁X₂X₃VX₄X₅X₆E
SEQ ID NO. 11: Truncated Vps10p-domain receptor tail
X₁X₂X₃VX₄X₅X₆EX₇X₈X₉X₁₀X₁₁X₁₂X₁₃X₁₄
SEQ ID NO. 12: Truncated Vps10p-domain receptor tail
KEQEMX₁X₂X₃VX₄X₅X₆EX₇X₈X₉X₁₀X₁₁X₁₂X₁₃X₁₄
SEQ ID NO. 13: Truncated SorCS2 receptor tail
TX₂PVX₄HX₆E
SEQ ID NO. 14: Truncated SorCS1 receptor tail
IX₂PVX₄HX₆E
SEQ ID NO. 15: Truncated SorCS3 receptor tail
IX₂SVX₄QX₆E
SEQ ID NO. 16: Truncated SorCS2 receptor tail
KEQEMTX₂PVX₄HX₆E
SEQ ID NO. 17: Truncated SorCS1 receptor tail
KEQEMIX₂PVX₄HX₆E
SEQ ID NO. 18: Truncated SorCS3 receptor tail
KEQEMIX₂SVX₄QX₆E
SEQ ID NO. 19: Truncated SorCS2 receptor tail
TX2PVX₄HX₆EDVQGAVQG
SEQ ID NO. 20: Truncated SorCS1 receptor tail
IX₂PVX₄HX₆ESRPNVPQT
SEQ ID NO. 21: Truncated SorCS3 receptor tail
IX₂SVX₄QX₆ENAPKITLS
SEQ ID NO. 22: Truncated SorCS2 receptor tail
KEQEMTX₂PVX₄HX₆EDVQGAVQG
SEQ ID NO. 23: Truncated SorCS1 receptor tail
KEQEMIX₂PVX₄HX₆ESRPNVPQT
SEQ ID NO. 24: Truncated SorCS3 receptor tail
KEQEMIX₂SVX₄QX₆ENAPKITLS
SEQ ID NO. 25: Truncated SorCS2 receptor tail (activating)
TX₂PVX₄HX₆E
SEQ ID NO. 26: Truncated SorCS1 receptor tail (activating)
IX₂PVX₄HX₆E
SEQ ID NO. 27: Truncated SorCS3 receptor tail (activating)
IX₂SVX₄QX₆E
SEQ ID NO. 28: Truncated SorCS2 receptor tail (activating)
KEQEMTX₂PVX₄HX₆E
SEQ ID NO. 29: Truncated SorCS1 receptor tail (activating)
KEQEMIX₂PVX₄HX₆E
SEQ ID NO. 30: Truncated SorCS3 receptor tail (activating)
KEQEMIX₂SVX₄QX₆E
SEQ ID NO. 31: Truncated SorCS2 receptor tail (activating)
TX₂PVX₄HX₆EDVQGAVQG
SEQ ID NO. 32: Truncated SorCS1 receptor tail (activating)
IX₂PVX₄HX₆ESRPNVPQT
SEQ ID NO. 33: Truncated SorCS3 receptor tail (activating)
IX₂SVX₄QX₆ENAPKITLS
SEQ ID NO. 34: Truncated SorCS2 receptor tail (activating)
KEQEMTX₂PVX₄HX₆EDVQGAVQG
SEQ ID NO. 35: Truncated SorCS1 receptor tail (activating)
KEQEMIX₂PVX₄HX₆ESRPNVPQT
SEQ ID NO. 36: Truncated SorCS3 receptor tail (activating)
KEQEMIX₂SVX₄QX₆ENAPKITLS
SEQ ID NO. 37: Truncated SorCS2 receptor tail (activating)
TDPVDHDE
SEQ ID NO. 38: Truncated SorCS2 receptor tail (activating)
KEQEMTDPVDHDE
SEQ ID NO. 39: Truncated SorCS2 receptor tail (activating)
TDPVDHDEDVQGAVQG
SEQ ID NO. 40: Truncated SorCS2 receptor tail (activating)
KEQEMTDPVDHDEDVQGAVQG
SEQ ID NO. 41: Truncated SorCS2 receptor tail (activating)
YARAAARNARAEKEQEMTDPVDHDEDVQGAVQ
SEQ ID NO. 42: Truncated SorCS1 receptor tail (activating)
IDPVDHDE
SEQ ID NO. 43: Truncated SorCS1 receptor tail (activating)
KEQEMIDPVDHDE
SEQ ID NO. 44: Truncated SorCS1 receptor tail (activating)
IDPVDHDESRPNVPQT
SEQ ID NO. 45: Truncated SorCS1 receptor tail (activating)
KEQEMIDPVDHDESRPNVPQT
SEQ ID NO. 46: Truncated SorCS3 receptor tail (activating)
IDSVDQDE
SEQ ID NO. 47: Truncated SorCS3 receptor tail (activating)
KEQEMIDSVDQDE
SEQ ID NO. 48: Truncated SorCS3 receptor tail (activating)
IDSVDQDENAPKITLS
SEQ ID NO. 49: Truncated SorCS3 receptor tail (activating)
KEQEMIDSVDQDENAPKITLS
SEQ ID NO. 50: Truncated SorCS2 receptor tail (inactivating)
TX₂PVX₄HX₆E
SEQ ID NO. 51: Truncated SorCS1 receptor tail (inactivating)
IX₂PVX₄HX₆E
SEQ ID NO. 52: Truncated SorCS3 receptor tail (inactivating)
IX₂SVX₄QX₆E
SEQ ID NO. 53: Truncated SorCS2 receptor tail (inactivating)
KEQEMTX₂PVX₄HX₆E
SEQ ID NO. 54: Truncated SorCS1 receptor tail (inactivating)
KEQEMIX₂PVX₄HX₆E
SEQ ID NO. 55: Truncated SorCS3 receptor tail (inactivating)
KEQEMIX₂SVX₄QX₆E
SEQ ID NO. 56: Truncated SorCS2 receptor tail (inactivating)
TX₂PVX₄HX₆EDVQGAVQG
SEQ ID NO. 57: Truncated SorCS1 receptor tail (inactivating)
IX₂PVX₄HX₆ESRPNVPQT
SEQ ID NO. 58: Truncated SorCS3 receptor tail (inactivating)
IX₂SVX₄QX₆ENAPKITLS
SEQ ID NO. 59: Truncated SorCS2 receptor tail (inactivating)
KEQEMTX₂PVX₄HX₆EDVQGAVQG
SEQ ID NO. 60: Truncated SorCS1 receptor tail (inactivating)
KEQEMIX₂PVX₄HX₆ESRPNVPQT
SEQ ID NO. 61: Truncated SorCS3 receptor tail (inactivating)
KEQEMIX₂SVX₄QX₆ENAPKITLS
SEQ ID NO. 62: Truncated SorCS2 receptor tail (inactivating)
TAPVAHAE
SEQ ID NO. 63: Truncated SorCS2 receptor tail (inactivating)
KEQEMTAPVAHAE
SEQ ID NO. 64: Truncated SorCS2 receptor tail (inactivating)
TAPVAHAEDVQGAVQG
SEQ ID NO. 65: Truncated SorCS2 receptor tail (inactivating)
KEQEMTAPVAHAEDVQGAVQG
SEQ ID NO. 66: Truncated SorCS2 receptor tail (inactivating)
YARAAARNARAEKEQEMTAPVAHAEDVQGAVQ
SEQ ID NO. 67: Truncated SorCS1 receptor tail (inactivating)
IAPVAHAE
SEQ ID NO. 68: Truncated SorCS1 receptor tail (inactivating)
KEQEMIAPVAHAE
SEQ ID NO. 69: Truncated SorCS1 receptor tail (inactivating)
IAPVAHAESRPNVPQT
SEQ ID NO. 70: Truncated SorCS1 receptor tail (inactivating)
KEQEMIAPVAHAESRPNVPQT
SEQ ID NO. 71: Truncated SorCS3 receptor tail (inactivating)
IASVAQAE
SEQ ID NO. 72: Truncated SorCS3 receptor tail (inactivating)
KEQEMIASVAQAE
SEQ ID NO. 73: Truncated SorCS3 receptor tail (inactivating)
IASVAQAENAPKITLS
SEQ ID NO. 74: Truncated SorCS3 receptor tail (inactivating)
KEQEMIASVAQAENAPKITLS
SEQ ID NO. 75: Truncated SorCS3 receptor tail (inactivating)
KEQEMIASVAQAENAPKITLS

### REFERENCES

1. Lopez A. D., et al., Nat Med 4, 1241 (November 1998).

2. Glerup S., et al., Molecular psychiatry (Jul. 26, 2016).

3. Gu B., et al., Neuron 88, 484 (Nov. 4, 2015).

4. Plotkin J L., et al., Neuron 83, 178 (Jul. 2, 2014).

5. Song J. H., et al., Molecular neurobiology 52, 1477 (December 2015).

6. Shin M. K., et al., Neurosci Lett 595, 63 (May 19, 2015).

7. Han J. C., et al., The New England journal of medicine 359, 918 (Aug. 28, 2008).

8. Rios M., et al., Molecular endocrinology 15, 1748 (October 2001).

9. Kernie S. G., et al., The EMBO journal 19, 1290 (Mar. 15, 2000).

10. Yamanaka M., et al., Biomed Res-Tokyo 29, 147 (July 2008).

11. Nakagawa T., et al., Diabetes 49, 436 (March 2000).

12. Deinhardt K., et al., Sci Signal. 2011; 4(202).

13. Glerup S., et al., Neuron. 2014; 82(5):1074-87.

14. Willnow T. E., et al., Nat Rev Neurosci. 2008; 9(12):899-909.

15. Glerup S., et al., Handb Exp Pharmacol. 2014; 220:165-89.

16. Nielsen M. S., et al., EMBO J. 2001; 20(9):2180-90.

17. Nielsen M. S., et al., Traffic. 2008; 9(6):980-94.

18. Vaegter C. B., et al., Nat Neurosci. 2011; 14(1):54-61.

19. Chung et al. (2008) Cell Stem Cell. 2(2):113-7.

20. Meyer-Franke A, et al., Nature 256: 495 (1975)

21. Ji Y., et al., Nat Neurosci. 2005; 8(2):164-72.

22. Lu Y., et al., J Neurosci. 2011; 31(33):11762-71.

23. Nagappan G., et al., Trends Neurosci. 2005; 28(9):464-71.

24. Woo N. H., et al., Nat Neurosci. 2005; 8(8):1069-77.

25. Pang P T, et al., Science. 2004; 306(5695):487-91.

1. A compound comprising a peptide or peptide analogue (P₁) consisting of 8 to 76 amino acid residues, the 8 to 76 amino acid residues comprising or consisting of the sequence X₁X₂X₃VX₄X₅X₆E (SEQ ID NO: 9), wherein
(i) X₁ is threonine (T) or isoleucine
(ii) X₂ is selected from the group consisting of phosphoserine (J); aspartic acid (D), glutamic acid (E), alanine (A) and serine (S);
(iii) X₃ is proline (P);
(iv) X₄ is selected from the group consisting of phosphoserine (J), aspartic acid (D), glutamic acid (E), alanine (A) and serine (S).
(v) X₅ is histidine (H) or glutamine (Q); and
(vi) X₆ is selected from the group consisting of phosphoserine (J), aspartic acid (D), glutamic acid (E), alanine (A) and serine (S).

2. The compound according to claim 1, wherein P₁ comprises or consists of the sequence KEQEMX₁X₂X₃VX₄X_{S}X₆E (SEQ ID NO: 10).

3. The compound according to claim 1, wherein P₁ comprises or consists of the sequence X₁X₂X₃VX₄X₅X₆EX₇X₈X₉X₁₀X₁₁X₁₂X₁₃X₁₄ (SEQ ID NO: 11), wherein
(i) X₇ is selected from aspartic acid (D), asparagine (N), serine (S)
(ii) X₈ is selected from valine (V), arginine (R), alanine (A)
(iii) X₉ is proline (P) or glutamine (Q)
(iv) X₁₀ is selected from glycine (G), lysine (K), asparagine (N)
(v) X₁₁ is selected from alanine (A), isoleucine (I), valine (V)
(vi) X₁₂ is selected from valine (V), threonine (T), proline (P)
(vii) X₁₃ is glutamine (Q) or leucine (L)
(viii) X₁₄ is selected from Glycine (G), threonine (T), serine (S).

4. The compound according to claim 1, wherein P₁ comprises or consists of the sequence
(SEQ ID NO: 12)
KEQEMX₁X₂X₃VX₄X₅X₆EX₇X₈X₉X₁₀X₁₁X₁₂X₁₃X₁₄.

5. The compound according to claim 1, wherein P₁ consists of the sequence X₁X₂X₃VX₄X₅X₆E (SEQ ID NO: 9), and wherein X₁ is threonine (T), X₃ is proline (P), and X₅ is histidine (H), and P₁ is thus SEQ ID NO: 13.

6. The compound according to claim 0, wherein P₁ consists of the sequence KEQEMX₁X₂X₃VX₄X₅X₆E (SEQ ID NO: 10), and wherein X₁ is threonine (T), X₃ is proline (P), and X₅ is histidine (H), and Pᵢ is thus SEQ ID NO: 16.

7. The compound according to claim 0, wherein P₁ consists of the sequence X₁X₂X₃VX₄X₅X₆EX₇X₈X₉X₁₀X₁₁X₁₂X₁₃X₁₄ (SEQ ID NO: 11), and wherein X₁ is threonine (T), X₃ is proline (P), X₅ is histidine (H), X₇ is aspartic acid (D), X₈ is valine (V), X₉ is glutamine (Q), X₁₀ is glycine (G), X₁₁ is alanine (A), X₁₂ valine (V), X₁₃ is glutamine (Q), X₁₄ is Glycine (G), and P₁ is thus SEQ ID NO: 19.

8. The compound according to claim 0, wherein P₁ consists of the sequence KEQEMX₁X₂X₃VX₄X₅X₆EX₇X₈X₉X₁₀X₁₁X₁₂X₁₃X₁₄ (SEQ ID NO: 12), and wherein X₁ is threonine (T), X₃ is proline (P), X₅ is histidine (H), X₇ is aspartic acid (D), X₈ is valine (V), X₉ is glutamine (Q), X₁₀ is glycine (G), X₁₁ is alanine (A), X₁₂ valine (V), X₁₃ is glutamine (Q), X₁₄ is Glycine (G), and Pᵢ is thus SEQ ID NO: 22.

9. The compound according to claim 1, wherein P₁ comprises at least one of X₂, X₄, X₆ wherein
(i) X₂ is selected from the group consisting of aspartic acid (D), glutamic acid (E), phosphoserine (J),
(ii) X₄ is selected from the group consisting of aspartic acid (D), glutamic acid (E), phosphoserine (J), or
(iii) X₆ is selected from the group consisting of aspartic acid (D), glutamic acid (E), phosphoserine (J).

10. The compound according to claim 0, wherein P₁ comprises or consists of a sequence selected from any one of SEQ ID NO: 25 to 74.

11. The compound according to claim 0, wherein P₁ comprises or consists of SEQ ID NO: 37.

12. The compound according to claim 1, wherein P₁ comprises at least one of X₂, X₄, X₆ wherein X₂, X₄, and/or X₆ are alanine (A).

13. (canceled)

14. The compound according to claim 1, wherein P₁ comprises or consists of SEQ ID NO: 62.

15-21. (canceled)

22. The compound according to claim 1, wherein P₁ is conjugated to at least one moiety, such as to two conjugated moieties, (Y₁) and (Y₂), which are selected from the group consisting of a Cell Penetrating Peptide (CPP), an Albumin Binding Moiety (ABM), and a detectable moiety (Z).

23. (canceled)

24. The compound according to claim 22, wherein the conjugated moiety is a CPP.

25-39. (canceled)

40. A composition comprising the peptide or peptide analogue according to claim 1, or a polynucleotide encoding said peptide, or a vector-comprising said polynucleotide, or a host cell comprising said polynucleotide.

41-42. (canceled)

43. A method of treating a disease selected from the group consisting of: diseases of the nervous system; mental and behavioural disorders;
neurodevelopmental congenital malformations and chromosomal abnormalities; cardiovascular disorders including vascular syndromes of brain in cerebrovascular diseases; metabolic and eating disorders; and neoplastic disorders in a subject, comprising administering. the composition according claim 40 to said subject.

44-46. (canceled)

47. The method according to claim 43, wherein the disease is nervous system disease selected from the group consisting of Huntington's disease and Alzheimer's disease.

48-51. (canceled)

52. The method according to claim 43, wherein the nervous system disease is neuropathic pain.

53-61. (canceled)

62. The method according to claim 43, wherein the metabolic and eating disorder is selected for the group consisting of diabetes, obesity, insulin resistance, and anorexia nervosa.

63-81. (canceled)
